SEQUENCE LISTING (2 sequences)

SEQ ID NO: 1 - DNA, 796 bp, Artificial Sequence
  miR223 promotor
acttgtacagcttcacagggctccatgcttagaaggaccccacacttagtttaatgttctgctgtcatcatcttgatattcttaatttttaaataaagggcctatcgttttcattttttactgggccttgcaaattatgtagctggttctgtatgccaggagagaagttggaagtaaaatggtattccaggaccaggaggcattctggcagagtgaaagaacatgtgatttggagtccatggggatgggtttaaatttcagctttccactaatttgctttgtgatactgagtatttccttttatccctcagaggctctgtttctcaattttgactacgggttttttcattagataatgtctcagttctggtattccaggtttccctcaattattctgggaaaacctccttgacccacaggcagagcctagggcagccaggtgctttctactctctctctctctgcagcttggaaagttagtgtctgttgaaggtcagctgggagttggtggaggcagggcagtggcctgctactattgctgcagtagcagaccctttcacaacagcattgttttgtcattttgcatccagatttccgttggctaacctcagtcttatcttcctcatttctgtttcctgttgaagacaccaagggcccttcaaaacacagaagcttcttgctcacggcagaaagcccaattccatctggcccctgcaggttggctcagcactggggaatcagagtcccctccatgaccaaggcaccactccactgacagggatccaagcttgccacc

SEQ ID NO: 2 - protein, 390 aa, Homo sapiens
  p47phox protein
MGDTFIRHIALLGFEKRFVPSQHYVYMFLVKWQDLSEKVVYRRFTEIYEFHKTLKEMFPIEAGAINPENRIIPHLPAPKWFDGQRAAENRQGTLTEYCSTLMSLPTKISRCPHLLDFFKVRPDDLKLPTDNQTKKPETYLMPKDGKSTATDITGPIILQTYRAIANYEKTSGSEMALSTGDVVEVVEKSESGWWFCQMKAKRGWIPASFLEPLDSPDETEDPEPNYAGEPYVAIKAYTAVEGDEVSLLEGEAVEVIHKLLDGWWVIRKDDVTGYFPSMYLQKSGQDVSQAQRQIKRGAPPRRSSIRNAHSIHQRSRKRLSQDAYRRNSVRFLQQRRRQARPGPQSPGSPLEEERQTQRSKPQPAVPPRPSADLILNRCSESTKRKLASAV

(19) United States
(12) Patent Application Publication
(10) Pub. No.: US 2023/0220416 A1
(43) Pub. Date: Jul. 13, 2023

(54) NOVEL TRANSDUCTION ENHANCERS AND USES THEREOF

(71) Applicant: Universität Zürich, (US)

(72) Inventors: Oleksandr PASTUKHOV, Zürich (CH); Ulrich SILER, Leymen (FR); Janine REICHENBACH, Zürich (CH)

(73) Assignee: Universität Zürich, Zurich (CH)

(21) Appl. No.: 17/926,757

(22) PCT Filed: Apr. 7, 2021
(86) PCT No.: PCT/EP2021/059069

§ 371 Date: Nov. 21, 2022

(30) Foreign Application Priority Data
May 27, 2020 (EP) 20176940.3
Oct. 6, 2020 (EP) 20200387.7

(51) Int. Cl. C12N 15/86; A61K 31/7048; A61K 47/62; A61K 47/20; A61K 47/10; A61K 31/357

(52) U.S. Cl. CPC C12N 15/86; A61K 31/7048; A61K 47/62; A61K 47/20; A61K 47/10; A61K 31/357; C12N 2740/15041

(57) ABSTRACT

The present invention relates to a method for transducing a target cell, the method comprising the step of contacting a target cell with a retroviral vector and a compound capable of enhancing transduction efficiency or a combination of such compounds, wherein the target cell is pre- and/or co-stimulated by pre- and/or co-incubation with said transduction enhancing compound or a combination of transduction enhancing compounds prior to and/or during contacting the target cell with the retroviral vector.

The present invention relates to a method for transducing a target cell, the method comprising the step of contacting a target cell with a retroviral vector and a compound capable of enhancing transduction efficiency or a combination of such compounds, wherein the target cell is pre- and/or co-stimulated by pre- and/or co-incubation with said transduction enhancing compound or a combination of transduction enhancing compounds prior to and/or during contacting the target cell with the retroviral vector.

### BACKGROUND OF THE INVENTION

In gene addition gene therapy, virus-derived vectors are used to introduce corrective genes (termed transgenes) in form of a cDNA into cells that carry a genetic loss-of-function or a function limiting mutation leading to disease. This introduced cDNA copy of the mutated gene consists of the healthy (non-mutated) sequence of the defective gene. In treated cells, the activity of the introduced transgene compensates for the missing activity of the defective gene.

For gene therapy of diseases originating in the haematopoietic system (i.e. diseases of red blood cells, of platelets, and diseases of the immune system), and for diseases of skin the production of skin grafts from gene-added epidermal/keratinocyte stem cells, retroviral vectors are state-of-the-art. These vectors are used to treat haematopoietic stem cells (HSC) or epidermal stem cells (ESC). The process by which retroviral vectors are stably integrating into the recipient HSC or ESC genome to add the corrective cDNA (i.e. a transgene) is called transduction. After intravenous reinfusion of treated HSC into the patient, these HSC engraft the bone marrow, where the added corrective cDNA is transmitted to all daughter cells upon HSC proliferation and subsequent differentiation to different blood and immune system cells. Correspondingly, after transplantation of skin grafts produced from transgenic ESCs, transgenic skin is maintained.

Today, mainly self-inactivating lentiviral (HIV-based) vectors that are pseudotyped with vesicular stomatitis (VSV-G) envelope are used for gene addition into HSC (Cartier et al. (2009) Science, PMID: 19892975; Cavazzana-Calvo et al. (2010) Nature 467: 318-22; Aiuti et al. (2013) Science 341: 1233151).

Clinical success in gene therapy is mainly dependent on the efficiency of gene addition, i.e. the level of transgene insertion mediated by the retroviral vectors, and on the dose of corrected cells that can be administered to a patient. Efficiency of gene addition on a per cell basis is dependent on the fold excess of viral vectors over target cell used during the transduction process, termed multiplicity of infection (MOI). The result of a successful gene addition can be quantified by determination of the average number of integrated vector copies per cell in a cell population, termed vector copy number (VCN). The latter is directly dependent on the quality of the process of retroviral transduction, i.e. VCN is higher when optimal retroviral transduction conditions can be achieved. E.g. a VCN=0.5 indicates that, on average, every second cell in a population of transduced cells obtained one copy of the therapeutic gene. A VCN=2 corresponds to 2 copies of the therapeutic gene per cell, on average, in a population of transduced cells.

The retroviral transduction process comprises the following sequence of events: the contact of therapeutic viral-derived particles with cells in cell culture (ex vivo), the binding of viral particles to the cell surface of target cells, the introduction of the therapeutic retroviral RNA into the target cell, the reverse transcription of retroviral A to pro-viral double-stranded DNA comprising the therapeutic cDNA sequence, and the successful integration into the genome of the target cell. Cell culture conditions during ex vivo retroviral transduction are of paramount importance for the efficiency of transduction. Optimal cell culture conditions during transduction should 1) maintain cell identity (e.g. the sternness of HSC), 2) conserve the ability of the cells e.g. of HSC to engraft e.g. in the bone marrow upon reinfusion into the patient, and 3) allow for efficient transduction (i.e. lead to high levels of gene addition).

Different approaches have been proposed in the past to enhance retroviral transduction, focusing on (1) improving the contact of the retroviral vector and the target cell, and (2) retroviral entry into the target cell.

Thus, there is a need for novel compounds and novel approaches for increasing transduction efficiency of human cells by a gene therapy vector, particularly of lentiviral vectors encoding the therapeutic transgene of interest.

In particular, there is a need for compounds or combinations of compounds that result in increased transduction efficiencies.

Further, there is a need in the art for clinically safe compounds that increase the transduction efficiencies of gene therapy vectors.

The present invention provides such novel compounds and approaches.

### SUMMERY OF THE INVENTION

The present invention is characterized in the herein provided embodiments and claims. In particular, the present invention relates, inter alia, to the following embodiments:

1. A method for transducing a target cell, the method comprising the step of contacting a target cell with a retroviral vector and a compound capable of enhancing transduction efficiency or a combination of such compounds, wherein the target cell is pre- and/or co-stimulated by pre- and/or co-incubation with said transduction enhancing compound or a combination of transduction enhancing compounds prior to and/or during contacting the target cell with the retroviral vector.

2. The method of embodiment 1, wherein the pre-incubation period is between 0.5 hours and 10 hours, particularly between 1 hour and 5 hours, particularly 2 hours.

3. The method of embodiment 1 or embodiment 2, wherein the transduction enhancing compound is selected from the group consisting of Silibinin, Midostaurin. Amphotericin B, Nystatin, and Natamycin, or a combination thereof.

4. The method of embodiment 1 or embodiment 2, wherein the transduction enhancing compound is selected from the group consisting of Resveratrol, Everolimus and Prostaglandin E2, or a combination thereof.

5. The method of embodiment 3, wherein the final concentration of the transduction enhancing compound is between about 0.05 μm and 500 μM, particularly between 0.1 μM and 10 μM for Silibinin, Midostaurin, Amphotericin B and Natamycin, and between 50 μM and 150 μM for Nystatin.

6. The method of embodiment 1 or embodiment 2, wherein the transduction enhancing compound is a poloxamer-based polymer, preferably poloxamer synperonic F108, or poloxamer 407 with a molecular weight between 11 kDa and 15 kDa.

7. The method of embodiment 5, wherein the final concentration of the transduction enhancing compound is between about 50 μg/ml and 5.000 μg/ml.

8. The method of embodiment 1 or embodiment 2, wherein the transduction enhancing compound is a mixture of Deoxyribonucleosides comprising 2′-Deoxythymidine, 2′-Deoxyadenosine, 2′-Deoxyguanosine and 2′-Deoxycytidine.

9. The method of embodiment 8, wherein the final concentration of each Deoxyribonucleoside is between about 0.1 mM and 10 mM of each deoxynucleoside.

10. The method of embodiment 1 or embodiment 2, wherein the transduction enhancing compound is a polymer selected from the group consisting of PEG-PCL-PEG polymer, PEG-PLGA-PEG polymer, and PEG-PLA-PEG polymer.

11. The method of embodiment 10, wherein the final concentration of the transduction enhancing compound is between about 20 μg/ml and 5000 μg/ml.

12. The method of embodiment 10 or embodiment 11, wherein a combination of transduction enhancing compounds is used comprising a polymer selected from the group consisting of PEG-PCL-PEG polymer, PEG-PLGA-PEG polymer, and PEG-PLA-PEG polymer and silibinin, particularly in a final concentration of between about 0.1 μM and 25 μM of silibinin and of between about 20 μg/ml and 5′000 μg/ml of the polymer.

13. The method of embodiment 10 or embodiment 11, wherein a combination of transduction enhancing compounds is used comprising a polymer selected from the group consisting of PEG-PCL-PEG polymer, PEG-PLGA-PEG polymer, and PEG-PLA-PEG polymer and midostaurin, particularly in a final concentration of between about 0.05 μM and 20 μM of midostaurin and of between about 20 μg/ml and 5000 μg/ml of the polymer.

14. The method of embodiment 10 or embodiment 11, wherein a combination of transduction enhancing compounds is used comprising a polymer selected from the group consisting of PEG-PCL-PEG polymer, PEG-PLGA-PEG polymer, and PEG-PLA-PEG polymer and amphotericin B, particularly in a final concentration of between about 0.1 μM and 20 μM of amphotericin B and of between about 20 μg/ml and 5′000 μg/ml of the polymer.

15. The method of embodiment 10 or embodiment 11, wherein a combination of transduction enhancing compounds is used comprising a polymer selected from the group consisting of PEG-PCL-PEG polymer, PEG-PLGA-PEG polymer, and PEG-PLA-PEG polymer and Nystatin, particularly in a final concentration of between about 5 μM and 500 mM of Nystatin and of between about 20 μg/ml and 5′000 μg/ml of the polymer.

16. The method of embodiment 10 or embodiment 11, wherein a combination of transduction enhancing compounds is used comprising a polymer selected from the group consisting of PEG-PCL-PEG polymer, PEG-PLGA-PEG polymer, and PEG-PLA-PEG polymer and Natamycin, particularly in a final concentration of between about 0.1 μM and 20 μM of Natamycin and of between about 20 μg/ml and 5′000 μg/ml of the polymer.

17. The method of embodiment 9, 10 or embodiment 11, wherein a combination of transduction enhancing compounds is used comprising a polymer selected from the group consisting of PEG-PCL-PEG polymer, PEG-PLGA-PEG polymer, and PEG-PLA-PEG polymer and a mixture of Deoxyribonucleosides comprising 2′-Deoxythymidine, 2′-Deoxyadenosine, 2′-Deoxyguanosine and 2′-Deoxycytidine, particularly in a final concentration of between about 0.1 mM and 10 mM of each Deoxyribonucleoside and of between about 20 μg/ml and 5000 μg/ml of the polymer.

18. The method of any one of embodiments 10 to 17, wherein the polymer is a functionalized polymer, in which one or both ends of the polymer are covalently linked to a cationic group, selected from the group consisting of an amino group, lysin, arginine, and histidine.

19. The method of embodiment 18, wherein the cationic group consisting of lysin, arginine, and/or histidine is present as a monomer or as a polymer.

20. The method of any one of embodiments 1 to 19, wherein the target cell is a cell selected from the group consisting of a lymphocyte, a tumor cell, a lymphoid lineage cell, a neuronal cell, an epithelial cell, an endothelial cell, a primary cell, a T-cell, a haematopoietic cell, and a stem cell.

21. The method of embodiment 20, wherein the target cell is a haematopoietic cell of human origin.

22. The method of embodiment 20 or embodiment 21, wherein the target cell is a haematopoietic stem cell.

23. The method of embodiment 20 or embodiment 21, wherein the target cell is CD34+ cell or a CD34+ cell enriched cell population. 24. The method of embodiment 20, wherein the target cell is a T-cell.

25. The method of embodiment 24, wherein the target cell is a T-cell defined by surface presentation of CD3, CD4 and/or CD8.

26. The method of any one of embodiments 1-7, 10-16 and 18 and 19, wherein the target cell is an enriched population of monocyte, macrophage, tissue resident macrophage or a microglial cell, a microglia like cell, or a dendritic cell.

27. The method of any one of embodiments 1 to 26, wherein the retroviral vector is a lentiviral vector.

28. The method of embodiment 27, wherein the lentiviral vector is a self-inactivating lentiviral vector.

29. The method of any one of embodiments 1 to 28, wherein the vector comprises a transgene under control of the miR223 promoter.

30. The method of any one of embodiments 1 to 29, wherein the vector comprises a p47phox, gp91phox, p22phox, p67phox or p40phox protein encoding cDNA in whole or in part.

31. A method for transducing a target cell, the method comprising the step of contacting a target cell with a retroviral vector and a compound capable of enhancing transduction efficiency or a combination of such compounds, wherein the target cell is pre-stimulated by pre-incubation with said transduction enhancing compound or a combination of transduction enhancing compounds prior to contacting the target cell with the retroviral vector.

32. A method for transducing a target cell, the method comprising the step of contacting a target cell with a retroviral vector and a compound capable of enhancing transduction efficiency or a combination of such compounds, wherein the target cell is co-stimulated by co-incubation with said transduction enhancing compound or a combination of transduction enhancing compounds upon and during contacting the target cell with the retroviral vector.

33. The method for transducing a target cell according to embodiment 31 and embodiment 32, wherein the transduction enhancing compound is selected from the group consisting of Silibinin, Midostaurin. Amphotericin B, Nystatin, Natamycin, or a combination thereof.

34. The method for transducing a target cell according to embodiment 31 or embodiment 32, wherein the transduction enhancing compound is selected from the group consisting of Resveratrol, Everolimus and Prostaglandin E2, or a combination thereof.

35. The method for transducing a target cell according to embodiment 31 or embodiment 32, wherein the transduction enhancing compound is a poloxamer-based polymer, preferably poloxamer synperonic F108, or poloxamer 407 with a molecular weight between 11 kDa and 15 kDa.

36. The method for transducing a target cell according to embodiment 31 or embodiment 32 wherein the transduction enhancing compound is a mixture of Deoxyribonucleosides comprising 2′-Deoxythymidine, 2′-Deoxyadenosine, 2′-Deoxyguanosine and 2f-Deoxycytidine.

37. The method for transducing a target cell according to embodiment 31 or embodiment 32, wherein the transduction enhancing compound is a polymer selected from the group consisting of PEG-PCL-PEG polymer, PEG-PLGA-PEG polymer, and PEG-PLA-PEG polymer.

38. The method of embodiment 37, wherein a combination of transduction enhancing compounds is used comprising a polymer selected from the group consisting of PEG-PCL-PEG polymer, PEG-PLGA-PEG polymer, and PEG-PLA-PEG polymer and

(i) silibinin, particularly in a final concentration of between about 0.1 μM and 25 μM; or midostaurin, particularly in a final concentration of between about 0.05 μM and 20 μM; or
(iii) amphotericin B, particularly in a final concentration of between about 0.1 μM and 20 μM; or
(iv) Nystatin, particularly in a final concentration of between about 5 μM and 5 mM; or (v) Natamycin, particularly in a final concentration of between about 0.1 μM and 20 μM, or
(vi) mixture of Deoxyribonucleosides comprising 2′-Deoxythymidine, 2′-Deoxyadenosine, 2′-Deoxyguanosine and 2′-Deoxycytidine, particularly in a final concentration of between about 0.1 mM and 10 mM of each Deoxyribonucleoside.

39. The method for transducing a target cell according to embodiment 31 or embodiment 32, wherein the transduction enhancing compound is a mixture of Everolimus and Amphotericin B.

40. The method of embodiment 39, wherein amphotericin B is present in a final concentration of between about 0.1 μM and 20 μM, and everolimus in a final concentration of between about 0.1 μM and 20 μM.

41. The method of embodiment 31, wherein the pre-incubation period is between 0.5 hours and 10 hours, particularly between 1 hour and 5 hours, particularly 2 hours.

42. The method of embodiment 32, wherein the co-incubation period is between 8 hours and 48 hours, particularly between 10 hours and 24 hours, but particularly 12 hours.

43. A method for treating a disease or disorder comprising transducing a retroviral therapeutic vector ex vivo or in vivo into hematopoietic stem cells and/or a population of enriched CD34-positive bone marrow cells, wherein the transduction is carried out with a method according to any one of embodiments 1 to 41.

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred embodiments of compositions, methods and materials are described herein. For the purposes of the present invention, the following terms are defined below.

“a,” “an,” and “the” are used herein to refer to one or to more than one o at least one, or to one or more) of the grammatical object of the article.

“or” should be understood to mean either one, both, or any combination thereof of the alternatives.

“and/or” should be understood to mean either one, or both of the alternatives.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

The terms “include” and “comprise” are used synonymously. “preferably” means one option out of a series of options not excluding other options. “e.g.” means one example without restriction to the mentioned example. By “consisting of is meant including, and limited to, whatever follows the phrase “consisting of”.

Reference throughout this specification to “one embodiment,” “an embodiment,” “a particular embodiment,” “a related embodiment,” “a certain embodiment,” “an additional embodiment,” “a specific embodiment” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

The present invention provides a method for transducing a target cell, the method comprising a step of contacting a target cell with a retroviral vector and a compound capable of enhancing transduction efficiency or a combination of such compounds, wherein the target cell is pre-and/or co-stimulated by pre- and/or co-incubation with said transduction enhancing compound or a combination of transduction enhancing compounds prior to and/or during contacting the target cell with the retroviral vector.

The method according to the invention may be performed in vivo or ex vivo. In certain embodiments, the method is performed ex vivo.

That is, the present invention is based, at least in part, on the finding that the transduction efficiency of a target cell with a retroviral vector can be increased by incubating the target cell with a transduction enhancing compound or a mixture of transduction enhancing compounds. For that, the target cell may be pre- and/or co-stimulated with the transduction enhancer or the combination of transduction enhancers disclosed herein.

The term “transduction enhancer” refers to any compound which upon presence during transduction results in an increase in VCN in comparison to its absence.

In certain embodiments, a target cell is co-stimulated with a transduction enhancer or a combination of transduction enhancers during the transduction step. Within the present invention, a target cell is said to be co-stimulated with a transduction enhancer or a combination of transduction enhancers, if the target cell is incubated in the presence of a transduction enhancer or a combination of transduction enhancers while said target cell is contacted with a retroviral vector.

It is preferred that during the co-stimulation step, the target cell, the retroviral vector and the transduction enhancer or the combination of transduction enhancers are contacted in a co-incubation step in a liquid medium, more preferably in a liquid cell culture medium.

The target cell may be co-incubated with the viral vector and the transduction enhancer or the combination of transduction enhancers for any amount of time. However, it is preferred that the target cell is co-incubated with the viral vector and the transduction enhancer or the combination of transduction enhancers for a period between about 8 hours and about 48 hours, particularly between about 10 hours and about 24 hours, but particularly about 12 hours.

Preferably, co-stimulation of a target cell with a transduction enhancer or a combination of transduction enhancers results in an increased susceptibility of the target cell for the retroviral vector.

In certain embodiments, a target cell may be pre-incubated with a transduction enhancer or a combination of transduction enhancers before the transduction step. Within the present invention, a target cell is said to be pre-stimulated with a transduction enhancer or a combination of transduction enhancers, if the target cell is incubated with a transduction enhancer or a combination of transduction enhancers before the target cell is contacted with a retroviral vector.

It is preferred that during the pre-stimulation step, the target cell and the transduction enhancer or the combination of transduction enhancers are contacted during an incubation step in a liquid medium, more preferably in a liquid cell culture medium.

The target cell may be pre-incubated with a transduction enhancer or a combination of transduction enhancers for any amount of time. However, it is preferred that the target cell is pre-incubated with a transduction enhancer or a combination of transduction enhancers for a period between about 0.5 hours and about 10 hours, particularly between about 1 hour and about 5 hours, but particularly 2 about hours.

Preferably, pre-stimulation of a target cell with a transduction enhancer or a combination of transduction enhancers results in an increased susceptibility of the target cell for a retroviral vector in a subsequent transduction step.

In certain embodiments, a target cell is pre-stimulated and co-stimulated with a transduction enhancer or a combination of transduction enhancers. That is, a target cell may first be pre-incubated with a transduction enhancer or a combination of transduction enhancers and, subsequently, be co-incubated with a retroviral vector and a transduction enhancer or a combination of transduction enhancers.

It has to be noted that the transduction enhancer or the combination of transduction enhancers may be identical or non-identical between the pre-stimulation and the co-stimulation step.

That is, in certain embodiments, a target cell may be pre-stimulated and co-stimulated with the same transduction enhancer or the same combination of transduction enhancers. For example, a target cell may first be pre-stimulated in a liquid medium comprising a transduction enhancer or a combination of transduction enhancers for a defined amount of time. To start the co-stimulation step, a retroviral vector may be added to the liquid medium comprising the target cell and the transduction enhancer or the combination of transduction enhancers. Alternatively, the target cell may be isolated from the pre-stimulation medium after a defined amount of time and may be transferred to fresh co-stimulation medium comprising the same transduction enhancer or the same combination of transduction enhancers and, optionally, a retroviral vector. That is, the co-incubation medium may already comprise the retroviral vector when the target cell is resuspended therein or the retroviral vector may be added after the target cell has been re-suspended in fresh co-incubation medium. The concentration and/or ratio of the transduction enhancer or the combination of transduction enhancers may be different or may be identical between the pre-stimulation medium and the co-stimulation medium.

In certain embodiments, the target cell may be contacted with a first transduction enhancer or a first combination of transduction enhancers in the pre-stimulation step and may then be contacted with a second transduction enhancer or a second combination of transduction enhancers in the co-stimulation step. For that, it is preferred that the target cell is pre-incubated in a medium comprising a first transduction enhancer or a first combination of transduction enhancers and may then be isolated from the pre-stimulation medium and transferred to a co-stimulation medium comprising a second transduction enhancer or a second combination of transduction enhancers and, optionally, a retroviral vector.

It is to be understood, that the pre-stimulation step not necessarily has to be directly followed by the co-stimulation step. That is, the cells may be incubated in a medium without a transduction enhancer between the pre-stimulation step and the co-stimulation step.

The efficiency of a transduction experiment may be determined as known in the art. Preferably, transduction efficiency may be measured by determining the vector copy number (VCN) in a single cell after a transduction experiment or by measuring the average VCN in a population of cells after a transduction experiment. “Vector copy number” or “VCN” refers to the number of copies of a vector, or portion thereof, in a cell's genome. The average VCN may be determined from a population of cells or from individual cell colonies. Exemplary methods for determining VCN include any form of polymerase chain reaction (PCR), such as qPCR or digital droplet PCR, and flow cytometry. For example, VCNs may be determined according to the method published by Charrier et al., Quantification of lentiviral vector copy numbers in individual hematopoietic colony-forming cells shows vector dose-dependent effects on the frequency and level of transduction, Gene Ther, 2011, 18(5), p. 479-487 or as described in Examples 1 or 5.

Several transduction enhancers and combinations of transduction enhancers reported herein result in an increase in transduction efficiency. An “increase in transduction efficiency” refers to an increase in VCN upon transduction of a cell population by a gene therapy vector in presence of a transduction enhancer compared to the absence of a transduction enhancer.

The target cell may be any cell that can be targeted with a retroviral vector. However, it is preferred that the target cell is a mammalian cell and, in particular a human cell.

In a particular embodiment, the invention relates to the method according to the invention, wherein the target cell is a cell selected from the group consisting of a lymphocyte, a tumor cell, a lymphoid lineage cell, a neuronal cell, an epithelial cell, keratinocytes, an endothelial cell, a primary cell, a T cell, a haematopoietic cell, and a stem cell.

The “target cell” may be a single cell of any of the cell types disclosed herein. However, it is to be understood that the method of the present invention may also be applied to a population of cells. That is, the target cell may be a homogenous population of cells, preferably any one of the cell types disclosed herein. However, the method of the invention may also be performed with a heterogeneous cell population, for example a cell population that has been obtained in an enrichment step. It is known in the art, that the enrichment of a specific cell type does not result in 100% pure cultures of said cell type. However, it is preferred that such a heterogeneous population of cells comprises at least one cell type that is disclosed herein.

The target cell may preferably be a mammalian cell and, more particularly, may be a cell from any germ layer, e.g. from the endoderm, the ectoderm or the mesoderm.

The term “endodermal cell” refers to a cell capable of differentiating into an endodermal organ, such as a liver, pancreas, intestinal tract, lung, thyroid, parathyroid, or urinary tract. The term “ectodermal cell” refers to a cell capable of differentiating into an ectodermal organ such as a brain, spinal cord, adrenal medulla, epidermis, hair/nail/dermal-gland, sensory organ, peripheral nerve, skin, or lens. The term “mesodermal cell”, as used herein, refers to a multipotent stem cell of mesodermal origin, and gives rise to the bone, cartilage, tendon, muscle, adipose tissue and vascular endothelium during development.

In certain embodiments, the target cell may be a fibroblast. The term “fibroblast”, as used herein, refers to a cell of mesenchymal origin. Fibroblasts are found in connective tissue. Fibroblasts synthesize actin-myosin filaments, the matrix elements (collagen, reticular and elastic fibers), and glycosaminoglycans and glycoproteins, which are secreted as amorphous intercellular substance. Fibroblasts include connective-tissue stem cells, matrix- and other protein-synthesizing cells, contractile cells, and phagocytic cells. Active fibroblasts are characterized by their abundant endoplasmic reticulum (ER), Golgi complex and ribosomes.

In certain embodiments, the target cell may be a smooth muscle cell or a non-smooth muscle cell.

In certain embodiments, the target cell may be an epithelial cell. The term “epithelial cell” as used herein refers to a cuboidal-shaped, nucleated cell covering the free surface (cutaneous, mucous or serous) of an organ or lining a tube or cavity in an animal body, and is consistent with the art-recognized definition of epithelial cells in epithelium. A layer of epithelial cells generally functions to provide a protective lining and/or surface that may also be involved in transport processes.

In certain embodiments, the target cell may be an endothelial cell. The term “endothelial cell” as used herein encompasses all endothelial cell types, such as the cells forming a single cell layer that lines all blood vessels and regulates exchanges between the bloodstream and the surrounding tissues. Many endothelial cell types exist and their phenotypes vary between different organs, between different segments of the vascular loop within the same organ, and between neighboring endothelial cells of the same organ and blood vessel type. Non-limiting examples of such endothelial cells are: liver sinusoidal endothelial cells (LSEC), (micro)vascular endothelial cells from e.g. lung, heart, intestine, skin, retina, arterial endothelial cells, such as endothelial cells from pulmonary artery, the aorta, umbilical artery and umbilical vein, extrahepatic endothelial cells from certain vascular beds, blood-brain barrier ECs, bone marrow ECs, and high endothelial venule cells (HEVs).

In certain embodiments, the target cell may be a neuronal cell. As used herein, the term “neuronal cell” or “neuron” denotes nervous system cells that include a central cell body or soma, and two types of extensions or projections: dendrites, by which, in general, the majority of neuronal signals are conveyed to the cell body, and axons, by which, in general, the majority of neuronal signals are conveyed from the cell body to effector cells, such as target neurons or muscle. Neurons can convey information from tissues and organs into the central nervous system (afferent or sensory neurons) and transmit signals from the central nervous systems to effector cells (efferent or motor neurons). Other neurons, designated interneurons, connect neurons within the central nervous system (the brain and spinal column). Certain specific examples of neuron types that may be subject to either ex or in vivo or a combination of ex and in vivo treatments or methods, according to the invention include cerebellar granule neurons, dorsal root ganglion neurons, and cortical neurons or any other cell type of the central or peripheral nerve system.

In certain embodiments, the target cell may be a tumor cell. The term “tumor cell” as used herein refers to a cell that is neoplastic. A tumor cell can be benign, i.e. one that does not form metastases and does not invade and destroy adjacent normal tissue, or malignant, i.e. one that invades surrounding tissues, is capable of producing metastases, may recur after attempted removal, and is likely to cause death of the host. Preferably a tumor cell that is subjected to a method of the invention may be derived form any germ layer (endoderm, ectoderm, mesoderm). In particular the tumor cell may be an epithelial-, a haematopoietic-, a germ-cell-or a mesenchymal-derived tumor cell, such as, without limitation, a tumor cell derived from skin cells, lung cells, intestinal epithelial cells, colon epithelial cells, testes cells, breast cells, prostate cells, brain cells, bone marrow cells, blood lymphocytes, ovary cells, gonadal and extragonadal related cells or thymus cells.

In certain embodiments, the target cell may a cell from the lymphoid lineage or a cell from the myeloid lineage. A cell from the lymphoid lineage is a cell that is derived from a common lymphoid progenitor, such as a natural killer cell, a T cell or a B cell. A cell from the myeloid lineage is a cell that is derived from a common myeloid progenitor, such as a megakaryocyte, a thrombocyte, an erythrocyte, a mast cell, a myeloblast, a basophil, a neutrophil, an eosinophil, a monocyte or a macrophage.

The term “primary cell” as used herein is known in the art to refer to a cell that has been isolated from a tissue and has been established for growth in vitro. Corresponding cells have undergone very few, if any, population doublings and are therefore more representative of the main functional component of the tissue from which they are derived in comparison to continuous cell lines thus representing a more representative model to the in vivo state.

Methods to obtain samples from various tissues and methods to establish primary cell lines are well-known in the art (see e.g. Jones and Wise, Methods Mal Biol. 1997). Primary cells for use in the method of the invention are derived from, e.g. bone marrow, blood, skin, lymphoma and epithelial tumors.

In certain embodiments, the target cell may be a lymphocyte. The term “lymphocyte” as used herein has the normal meaning in the art, and refers to any of the mononuclear, non-phagocytic leukocytes, found in the blood, lymph, and lymphoid tissues, i.e., cells, B cells and T cells.

In a particular embodiment, the invention related to the method according to the invention, wherein the target cell is a T cell. The term “T cell” as used herein refers to a type of lymphocyte that plays a central role in cell-mediated immunity. T cells, also referred to as T lymphocytes, can be distinguished from other lymphocytes, such as B cells and natural killer cells, by the presence of a T cell receptor (TCR) on the cell surface. There are several subsets of T cells with distinct functions, including but not limited to, T helper cells, cytotoxic T cells, memory T cells, regulatory T cells and natural killer T cells. In certain embodiments, the target cell may be a T cell defined by surface expression of CD3, CD4 and/or CD8.

Thus, in certain embodiments, the target cell may be a T cell that is characterized by the expression of CD3. The term “CD3” as used herein refers to all mammalian species, preferably human, of the cluster of differentiation 3 (CD3) T cell co-receptor. In mammals, CD3 comprises a CD3 ζ chain, a CD3 delta chain and two CD3 epsilon chains. Accordingly, the target cell of the invention may be any T cell that expresses the T cell co-receptor in addition to a T cell receptor.

In certain embodiments, the target cell may be a T cell that is characterized by the expression of CD4. The term “CD4”, as used herein, refers to a cluster of differentiation 4, a glycoprotein expressed on the surface of T helper cells, monocytes, macrophages, and dendritic cells. CD4 is a co-receptor that assists the T cell receptor (TCR) with an antigen-presenting cell. Thus, in certain embodiments, the target cell my be a T helper cell.

In certain embodiments, the target cell may be a T cell that is characterized by the expression of CD8. The term “CD8”, as used herein, refers to the cluster of differentiation 8, a transmembrane glycoprotein that serves as a co-receptor for the T cell receptor (TCR) expressed in the cytotoxic T cells (CTL). Thus, in certain embodiments, the target cell my be a cytotoxic T cell.

In certain embodiments, the method of the invention may be used in the production of CAR T, CAR M or CAR NK cells. That is, a T cell, in particular a cytotoxic CD8+ T cell or a CD4+T helper cell, monocytes, macrophages or NK cells may be pre-stimulated and/or co-stimulated with the transduction enhancer or the combination of transduction enhancers of the invention as disclosed herein. During the co-stimulation step, the T cell, monocytes, macrophages of NK cell may then be contacted with a retroviral vector, in particular a lentiviral vector, comprising a nucleic acid encoding a chimeric antigen receptor (CAR).

In a particular embodiment, the invention relates to the method according to the invention, wherein the target cell is a haematopoietic cell, in particular a haematopoietic cell of human origin. The term “hematopoietic cell” as used herein refers to any type of cell of the hematopoietic system, including, but not limited to, undifferentiated cells such as hematopoietic stem cells and progenitor cells, and differentiated cells e.g. leukocytes (for example granulocytes, monocytes, NK cells and lymphocytes).

In a particular embodiment, the invention relates to the method according to the invention, wherein the target cell is a haematopoietic stem cell. The term “hematopoietic stem cell” is used in the broadest sense to refer to stem cells from which blood cells derive, including pluripotent stem cells, lymphoid and myeloid stem cells.

Haematopoietic stem cells may develop to any cell lineage present in blood or in tissue. This term, as used herein, refers both to the earliest renewable hematopoietic cell populations responsible for generating cell mass in the blood (e.g., CD34−/CD133+, CD34−/AC133−/Lineage−, CD34+/AC133+ cells, e.g. Lineage−CD34+CD38−CD90+CD45RA- (Majeti R., Park C. Y. & Weissman I. L. (2007) Cell Stem Cell 1: 635-645), lineage- CD133+CD38-CD33- (Götz et al. (2007) Exp Hemat. 35: 1408-14)), and the very early hematopoietic progenitor cells (HSPCs), which are somewhat more differentiated, yet are not committed and can readily revert to become a part of the earliest renewable hematopoietic cell population (e.g., CD34+ cells, especially CD34+CD38-cells). In healthy humans, most of the hematopoietic pluripotent stem cells, and the lineage committed progenitor cells are CD34+. The majority of these cells are CD34+CD38+, with a minority of cells (<10%) being CD34+CD38−. The CD34+CD38− stem cell fraction comprises the most immature hematopoietic cells, which are capable of self-renewal and multilineage differentiation. This fraction contains more long-term culture initiating cells (LTC-IC) and exhibits longer maintenance of the sternness and delayed proliferative response to cytokines, as compared to cells of the CD34+CD38+ cell fraction. Preferably, certain embodiments are applied to cell populations enriched in human CD34+ cells.

In certain embodiments, the target cell is a hematopoietic progenitor cell. The term “hematopoietic progenitor cell” refers to the progeny of a pluripotent hematopoietic stem cell which are committed for a particular line of differentiation or to any cell population comprising pluripotent hematopoietic stem cells capable of self-renewal and multilineage differentiation. These committed progenitor cells are irreversibly determined as ancestors of only one or a few blood cell types, e.g. erythrocytes, megakaryocytes, monocytes or granulocytes.

In certain embodiments, the target cell is a hematopoietic precursor cell. The term “hematopoietic precursor cell” as used herein includes hematopoietic stem cells, hematopoietic progenitor cells or any cell which gives rise to a cell in the hematopoietic lineages (e.g., lymphoid, myeloid). Examples of hematopoietic precursor cells are CFU-GEMM (colony forming unit-granulocyte-erythrocyte-megakaryocyte-monocyte), CFU-GM (colony forming unit-granulocyte-monocyte), CFU-E (colony forming unit-erythrocyte), BFU-E (burst forming unit-erythrocyte), CFU-G (colony forming unit-granulocyte), CFU-eo (colony forming unit-eosinophil), and CFU-Meg (colony forming unit-megakaryocyte).

In a particular embodiment, the invention relates to the method according to the invention, wherein the target cell is a CD34+ cell or a cell comprised in a CD34+enriched cell population. The term “CD34”, as used herein, refers to a cluster of differentiation present on certain cells within the human body. It is a cell surface glycoprotein and functions as a cell-cell adhesion factor. It may also mediate the attachment of stem cells to bone marrow extracellular matrix or directly to stromal cells. Cells expressing CD34 (CD34+ cell) are normally found in the umbilical cord and bone marrow as hematopoietic cells, a subset of mesenchymal stem cells, endothelial hematopoietic progenitor cells, endothelial cells of blood vessels, but not lymphatic cells. Accordingly, the term “CD34+ cells” as used herein preferably refers to hematopoietic stem and progenitor cells derived from human bone marrow that “are positive for” i.e., “express”, the hematopoietic stem cell antigen CD34. Further, the target cell may be any cell that is comprised in a CD34+ enriched cell population. The skilled person is aware of methods to enrich CD34+ cells. Further, commercial kits for the enrichment of CD34+ cell populations are available. Certain embodiments may be applied to cell populations enriched in human CD34+ cells.

In a particular embodiment, the invention relates to a method according to the invention, wherein the target cell is a monocyte, a macrophage, a tissue resident macrophage, a microglial cell or a dendritic cell.

That is, in certain embodiments, the target cell may be a monocyte or a cell comprised in an enriched population of monocytes. The term “monocyte”, as used herein, refers to a type of white blood cells that have two main functions in the immune system: (1) replenish resident macrophages and dendritic cells under normal states, and (2) in response to inflammation signals, monocytes can move quickly (approx. 8-12 hours) to sites of infection in the tissues and differentiate into macrophages and dendritic cells to elicit an immune response. Half of them are stored in the spleen. Monocytes are usually identified in stained smears by their large bilobate nucleus. In addition to the expression of CD14, monocytes also show expression of one or more of the following surface markers 125I-WVH-1, 63D3, Adipophilin, CB12, CD11a, CD11b, CD14, CD16, CD54, CD163, cytidine deaminase, Flt-1, and the like. Methods and commercial kits for the enrichment of monocytes are known in the art.

In certain embodiments, the target cell may be a macrophage or a cell comprised in an enriched population of macrophages. The term “macrophage”, as used herein, refers to CD14+ positive cells derived from the differentiation of the monocytes characterized in that they are phagocytes, acting in both non-specific defense (innate immunity) as well as to help initiate specific defense mechanisms (adaptive immunity) of vertebrate animals. Their role is to phagocytose (engulf and then digest) cellular debris and pathogens either as stationary or as mobile cells, and to stimulate lymphocytes and other immune cells to respond to the pathogen.

In certain embodiments, the macrophage may be a tissue-resident macrophage, such as a resident macrophage e.g. in the brain or the kidney. In addition to the expression of CD14, macrophages also show expression of one or more of the following surface markers: CD11b, F4/80(mice)/EMR1(human), Lysozyme M, MAC-1/MAC-3, 27E10, Carboxypeptidase M, Cathepsin K, CD163, CD86, CD206, CD209, Mer and CD68. These markers can be determined by flow cytometry or immunohistochemical staining. Methods and commercial kits for the enrichment of macrophages are known in the art. It has to be noted that certain types of tissue-resident macrophages may not be derived from monocytes, but from other cell types or tissues, such as the yolk sack. However, such non-monocyte derived tissue-resident macrophages may also be used in the method of the invention.

In certain embodiments, the target cell may be a dendritic cell, in particular a myeloid dendritic cell, or a cell comprised in an enriched population of dendritic cells, in particular myeloid dendritic cells. The term “myeloid dendritic cell”, as used herein, refers to a population of dendritic cells which derive from monocytes and which include, without limitation, mDC-1 and mDC-2. In addition to the expression of CD14, myeloid dendritic cells also show expression of one or more of the following surface markers: Thrombomodulin/CD141/BDCA-3, CD1 c/BDCA-1, Neuropilin-1/BDCA-4, DC-. SIGN/CD209, SIRPa/CD172a, ADAM19, BDCA-2, CD1a, CD11c, CD21, CD86, CD208, Clusterin, Estrogen Receptor-alpha. Methods and commercial kits for the enrichment of macrophages are known in the art.

In certain embodiments, the target cell may be a microglia cell, or a cell comprised in an enriched population of microglia cells. The term “microglia” as used herein refers to the smallest of the glial cells that can act as phagocytic cells, cleaning up CNS-localized debris. They are considered to be a type of immune cell found in the brain and were characterized by Iba1, CD11b, CD45, CD11c, Ferritin, CD68, TMEM2 and/or CD33 expression (Hopperton et al. (2018) Mol. Psych 23: 177-198). Microglia are close relatives of other phagocytic cells including macrophages and dendritic cells. Like macrophages, microglia are derived from myeloid progenitor cells from the bone marrow.

In a certain embodiment, the target cell may be a microglia or a microglia-like cell, or a cell comprised in a enriched population of microglia like cells. The term “microglia-like cells” refers to blood derived monocytes/macrophages capable of crossing the blood-brain barrier, especially if infused into a patient after busulfan or treosulfan conditioning. Microglia-like cells have been reported to enter the brain during neuroinflammatory conditions (Mendiola A. S. et al. (2020) Nat Immunol 21: 513-524, PMID 32284594) and upon brain metastasis progression (Schulz M. Et al.(2020) iScience 23: 101178. doi: 10.1016/j.isci.2020.101178.), thereby facilitating phagocytosis and innate immune functions comparable and/or complementary to the activity of brain tissue resident microglia.

Various methods are known in the art to identify and/or enrich any of the cell types listed above. For example, cells of a specific cell type may be enriched by flow cytometry based on the expression of specific cell surface markers or combinations of cell surface markers. Further, cells of a specific cell type may be identified by various microscopy methods known in the art or based on their cytokine secretion profile. Various commercial kits exist for the identification and/or enrichment of specific cell types.

The method of the invention may be used to improve the transduction of a target cell with a retroviral vector.

The term “vector” is used herein to refer to a nucleic acid molecule capable transferring or transporting another nucleic acid molecule. As will be evident to one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell, or to a viral particle that mediates nucleic acid transfer. Viral particles will typically include various viral components and sometimes also host cell components in addition to nucleic acid(s).

The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid used in plasmidic form for transient cell transfection of a cell for virus production or used upon stable integration into the genome of a cell for the generation of a stable virus producing cells containing structural and functional genetic elements, or portions thereof, that are primarily derived from a retrovirus.

As used herein, the term “retrovirus” refers to an RNA virus that reversely transcribes its genomic A into a linear double-stranded DNA copy and subsequently covalently integrates its genomic DNA into a host genome. Retroviruses are a common tool for gene delivery (Miller, 2000, Nature. 357: 455-460). Once the virus is integrated into the host genome, it is referred to as a “provirus.” The provirus serves as a template for RNA polymerase II and directs the expression of viral A molecules encoded by the host cell. Illustrative retroviruses include, but are not limited to: Moloney murine leukemia virus (MoMLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus including foamy virus, Friend murine leukemia virus (FMLV), Murine Stem Cell Virus (MSCV) and Rous Sarcoma Virus (RSV), alpha-retrovirus and lentivirus. That is, the retroviral vector used in the method of the invention may be derived from any retrovirus discloses herein. Furthermore, the term “retrovirus” refers to any pseudotyped retroviral particles, e.g. comprising vesicular stomatitis virus glycoprotein (VSV-G) pseudotyped retroviral particles or an envelope decorated with syncytin-related proteins preferably syncytin-2 protein (Esnault C. et al. (2008) PNAS 105: 17532-17537) and retroviral particles free from pseudotyping viral glycoproteins (Böker K. O. et al. (2018) Mol Ther. 26: 634-647).

In a particular embodiment, the invention relates to the method according to the invention, wherein the retroviral vector is a lentiviral vector.

The term “lentiviral vector” refers to a retroviral vector or plasmid containing structural and functional genetic elements, or portions thereof, including LTRs that are primarily derived from a lentivirus.

As used herein, the term “lentivirus” refers to a group (or genus) of complex retroviruses. Illustrative lentiviruses include, but are not limited to; HIV (human immunodeficiency virus; including HTV type 1, and HIV type 2); visna-maedi virus (VMV); the caprine arthritis-encephalitis virus (CAEV); equine infectious anemia virus (EIAV); feline immunodeficiency virus (FIV); bovine immune deficiency virus (BIV); and simian immunodeficiency virus (SW).

The term lentiviral vector further includes hybrid vectors. The term “hybrid” refers to a vector, LTR (long terminal repeat) or other nucleic acid containing both retroviral, e.g., lentiviral, sequences and non-lentiviral viral sequences. For example, a hybrid vector may refer to a vector or transfer plasmid comprising retroviral e.g., lentiviral, sequences for reverse transcription, replication, integration and/or packaging and alphavirus subgenomic promoter sequences, non-structural proteins, and/or polymerase recognition sites. Another example of hybrid vectors are pseudotyped lentiviral vectors comprising lentiviral elements for reverse transcription and integration, but covered by envelope proteins of different origin, e.g. covered by the vesicular stomatitis virus glycoprotein (VSV-G), or different viral envelope proteins.

In an additional embodiment, the invention relates to integration deficient retroviral vectors, comprising an inactive form of retroviral integrase enzyme used to provide “template DNA” to a cell for targeted genome editing by sequence-specific insertion of a single (nicking) and/or double strand break in the genome, in combination of provision of a template DNA, covering sequences flanking the position of single (nicking) and/or double strand break and the desired sequence in between termed “template DNA”, which can transiently be provided to a cell by said integration deficient retroviral vectors.

In a particular embodiment, the invention relates to the method according to the invention, wherein the lentiviral vector is a self-inactivating lentiviral vector.

“Self-inactivating” (SIN) vectors are replication-defective vectors, e.g., retroviral or lentiviral vectors, in which the right (3′) LTR enhancer-promoter region, known as the U3 region, has been modified (e.g., by deletion and/or substitution) to prevent viral transcription beyond the first round of viral replication. Consequently, the vectors are capable of infecting and then integrating into the host genome only once, and cannot be passed further. This is because the right (3′) LTR U3 region, harbouring a deletion of the viral promoter/enhancer sequence, is used as a template for the left (5′) LTR U3 region during viral reverse transcription and, thus, new viral transcripts from integrated SIN vectors cannot be made without the U3 enhancer-promoter. If the viral transcript is not made, it cannot be processed or packaged into virions, hence the life cycle of the virus ends. Accordingly, SIN vectors greatly reduce risk of creating unwanted replication-competent virus, since the right (3′) LTR U3 region has been modified to prevent viral transcription beyond the first round of replication, hence eliminating the ability of the virus to be passed.

In a further and/or alternative embodiment of the invention, the 3′ LTR may be modified such that the U5 region is replaced, for example, with a heterologous or synthetic poly(A) sequence, one or more insulator elements, and/or an inducible promoter. It should be noted that modifications to the LTRs such as modifications to the 3′ LTR, the 5′ LTR, or both 3′ and 5′ LTRs, are also included in the invention.

An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) thymidine kinase promoters. Typical promoters are able to drive high levels of transcription in a Tat-independent manner. This replacement reduces the possibility of recombination to generate replication-competent virus because there is no complete U3 sequence in the virus production system.

The term “long terminal repeat (LTR)” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals, including transcriptional control elements, polyadenylation signals and sequences needed for replication and integration of the viral genome. The U3 region contains the enhancer and promoter elements. The U5 region is the sequence between the primer binding site and the R region, and contains the polyadenylation sequence. The R (repeat) region is flanked by the U3 and U5 regions. On DNA level, the LTR composed of U3, R and U5 regions, appears at both the 5′ and 3′ ends of the viral genome. Adjacent to the 5′ LTR are sequences necessary for reverse transcription of the genome (the tRNA primer binding site) and for efficient packaging of viral RNA into particles (the Psi site).

Encompassed by the invention is also a method for transducing a target cell, the method comprising the step of contacting a target cell with a gene therapy vector and a compound capable of enhancing transduction efficiency or a combination of such compounds, wherein the target cell is pre- and/or co-stimulated by pre- and/or co-incubation with said transduction enhancing compound or a combination of transduction enhancing compounds prior to and/or during contacting the target cell with the gene therapy vector. It is to be understood that the combination of any gene therapy vector disclosed herein with any target cell disclosed herein and/or any transduction enhancer or combination of transduction enhancers disclosed herein is encompassed by the invention.

The term “gene therapy vector” includes all vectors used as vehicle to transport genetic information into target cells within a gene therapy approach in which the genetic information to be transported is pre-defined by gene therapy vector design. A gene therapy vector may be an adenoviral vector, an adeno-associated viral vector, a herpes viral vector, a foamy viral vector, or a retroviral vector, in particular wherein the retroviral vector is a lentiviral vector.

The gene therapy vector or retroviral vector of the invention may comprise a nucleotide sequence of interest that is intended to be transferred to a target cell. The nucleotide sequence of interest is not limiting within the present invention and may be any nucleotide sequence that may be transferred to a target cell. However, it is preferred that the nucleotide sequence of interest comprises a transgene and, more preferably, regulatory elements that are required for the expression of the transgene in the target cell.

The term “transgene” as used herein refers to particular nucleic acid sequences encoding a polypeptide or a portion of a polypeptide to be expressed in a cell into which the nucleic acid sequence is inserted, i.e., the target cell of the invention. Further, it is to be understood that a transgene may encode multiple polypeptides, for examples polypeptides making up a chimeric antigen receptor (CAR). However, it is also possible that transgenes are expressed as RNA, typically to lower the amount of a particular polypeptide in a cell into which the nucleic acid sequence is inserted. These RNA molecules include but are not limited to molecules that exert their function through RNA interference (shRNA, RNAi, micro-RNA regulation (miR), catalytic RNA, antisense RNA, RNA aptamers, long-noncoding RNAs, etc. Of note, expression of the transgene may be restricted to a subset of the cells into which the nucleic acid sequence is inserted. The term transgene is meant to include (1) a nucleic acid sequence that is not naturally found in the cell (i.e., a heterologous nucleic acid sequence); (2) a nucleic acid sequence that is a mutant form of a nucleic acid sequence naturally found in the cell into which it has been introduced; (3) a nucleic acid sequence that serves to add additional copies of the same (i.e., homologous) or a similar nucleic acid sequence naturally occurring in the cell into which it has been introduced; or (4) a silent naturally occurring or homologous nucleic acid sequence whose expression is induced in the cell into which it has been introduced; or (5) a sequence serving as “template DNA” for targeted homologous recombination upon gene editing by targeted insertion of a single and/or double strand break. By “mutant form” is meant a nucleic acid sequence that contains one or more nucleotides that are different from the wild-type or naturally occurring sequence, i.e., the mutant nucleic acid sequence contains one or more nucleotide substitutions, deletions, and/or insertions. In some cases, the transgene may also include a sequence encoding a leader peptide or signal sequence such that the transgene product will be secreted from the cell.

In certain embodiments, the transgene may be a nucleic acid encoding a naturally occurring polypeptide that is not expressed or expressed at reduced levels in the target cell due to a congenital or acquired genetic defect. In other embodiments, the transgene may encode a chimeric antigen receptor (CAR). When the transgene encodes a CAR, it is preferred that the target cell is a T cell, a monocyte or a macrophage or an NK cell.

In certain embodiments, the transgene may be operably linked to a promoter. The term “promoter” refers to nucleic acid sequences that regulate, either directly or indirectly, the transcription of corresponding nucleic acid coding sequences to which they are operably linked (e.g., a transgene). A promoter may function alone to regulate transcription or may act in concert with one or more other regulatory sequences (e.g., enhancers or silencers). In the context of the present application, a promoter is typically operably linked to a transgene to regulate transcription of the transgene.

The term “operably linked” as used herein refers to the arrangement of various nucleic acid molecule elements relative to each, such that the elements are functionally connected and are able to interact with each other. Such elements may include, without limitation, a promoter, an enhancer, a polyadenylation sequence, one or more introns, and a coding sequence of a gene of interest to be expressed (i.e., the transgene). The nucleic acid sequence elements, when properly oriented or operably linked, act together to modulate the activity of one another, and ultimately may affect the level of expression of the transgene. By modulate is meant increasing, decreasing, or maintaining the level of activity of a particular element. The position of each element relative to other elements may be expressed in terms of the 5′ terminus and the 3′ terminus of each element, and the distance between any particular elements may be referenced by the number of intervening nucleotides, or base pairs, between the elements. As understood by the skilled person, operably linked implies functional activity, and is not necessarily related to a natural positional link. Indeed, when used in a vector, the regulatory elements will typically be located immediately upstream of the promoter (although this is generally the case, it should definitely not be interpreted as a limitation or exclusion of positions within the vector), but this needs not be the case in vivo.

The promoter comprised in the retroviral vector or the gene therapy vector of the invention may be any promoter known in the art, preferably a promoter that can induce transcription of a transgene in the target cell of the invention. The promoter may be a naturally occurring promoter or a synthetic promoter. The promoter may be an ubiquitous promoter, i.e., a promoter that is active in a wide range of cells, tissues and cell cycles. Alternatively, the promoter may be a promoter that is active only in certain cell types or even a single cell type or that is active only at a certain stage of the cell cycle. Further, the promoter may be a constitutive promoter or a promoter for conditional expression.

As used herein, the term “constitutive promoter” refers to a promoter that continually or continuously allows for transcription of an operably linked sequence. Constitutive promoters may be an “ubiquitous promoter” that allows expression in a wide variety of cell and tissue types or a “tissue-specific promoter” that allows expression in a restricted variety of cell and tissue types. Illustrative ubiquitous promoters include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) thymidine kinase promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), 13-kinesin (β-KIN), the human ROSA 26 locus (Irions et al., Nature Biotechnology 25, 1477-1482 (2007)), an Ubiquitin C promoter (UBC), a phosphoglycerate kinase-1 (PGK) promoter, a cytomegalovirus enhancer/chicken β-actin (CAG) promoter, a β-actin promoter and U6 and HI shRNA promoters.

In a particular embodiment, it may be desirable to use a tissue-specific promoter to achieve cell type specific, lineage specific, or tissue-specific expression of a desired polynucleotide sequence (e.g., to express a particular nucleic acid encoding a polypeptide in only a subset of cell types or tissues or during specific stages of development). Illustrative examples of tissue specific promoters include, but are not limited to: a B29 promoter (B cell expression), a runt transcription factor (CBFa2) promoter (stem cell specific expression), a CD14 promoter (monocytic cell expression), a CD43 promoter (leukocyte and platelet expression), a CD45 promoter (hematopoietic cell expression), a CD68 promoter (macrophage expression), a CYP450 3A4 promoter (hepatocyte expression), a desmin promoter (muscle expression), an elastase 1 promoter (pancreatic acinar cell expression, an endoglin promoter (endothelial cell expression), a fibroblast specific protein 1 promoter (FSP1) promoter (fibroblast cell expression), a fibronectin promoter (fibroblast cell expression), a fms-related tyrosine kinase 1 (FLT1) promoter (endothelial cell expression), a glial fibrillary acidic protein (GFAP) promoter (astrocyte expression), an insulin promoter (pancreatic beta cell expression), an integrin, alpha 2b (ITGA2B) promoter (megakaryocytes), an intracellular adhesion molecule 2 (ICAM-2) promoter (endothelial cells), an interferon beta (IFN-p) promoter (hematopoietic cells), a keratin 5 promoter (keratinocyte expression), a myoglobin (MB) promoter (muscle expression), a myogenic differentiation 1 (MYOD1) promoter (muscle expression), a nephrin promoter (podocyte expression), a bone gamma-carboxyglutarnate protein 2 (OG-2) promoter (osteoblast expression), an 3-oxoacid CoA transferase 2B (Oxct2B) promoter, (haploid-spermatid expression), a surfactant protein B (SP-B) promoter (lung expression), a synapsin promoter (neuronal expression), a Wiskott-Aldrich syndrome protein (WASP) promoter (hematopoietic cell expression). In one embodiment, a vector of the present invention comprises a tissue specific promoter and/or enhancer that expresses a desired polypeptide in microglial cells, e.g., an MND promoter. In certain embodiments, the retroviral vector or the gene therapy vector of the invention may comprise a transgene under control of the miR223 promoter.

In certain embodiments, the promoter comprised in the retroviral vector may be any one of the vectors disclosed in EP 2 021 499 or in Santilli et al. (2010) Mol Ther 19: 122-32; PMID 20978475) or any vector comprising the chimeric promoter mentioned in PMID 20978475 and consisting a fused promoter sequences derived from cFES and cathepsin G promoter sequences.

As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue-specific expression. Certain embodiments of the invention provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

Conditional expression can also be achieved by using a site-specific DNA recombinase. According to certain embodiments of the invention the vector comprises at least one (typically two) site(s) for recombination mediated by a site-specific recombinase. As used herein, the terms “recombinase” or “site-specific recombinase” include excisive or integrative proteins, enzymes, co-factors or associated proteins that are involved in recombination reactions involving one or more recombination sites (e.g., two, three, four, five, seven, ten, twelve, fifteen, twenty, thirty, fifty, etc.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Illustrative examples of recombinases suitable for use in particular embodiments of the present invention include, but are not limited to: Cre, Int, IHF, Xis, Flp, Fis, Hin, Gin, ΦC31, Cin, Tn3 resolvase, TndX, XerC, XerD, TnpX, Hjc, Gin, SpCCE1, and ParA.

Various promoters have been described in the art and the skilled person is capable of identifying promoters that are particularly suited for a specific application. However, it has to be noted that neither the choice of the transgene, nor the choice of the promoter, are limiting features in the method claimed herein. Thus the nucleotide of interest comprised in the gene therapy vector or the retroviral vector may be any nucleotide, provided that the size of the nucleotide does not exceed the genetic load of the vector.

The method of the invention may be used for increasing the transduction efficiency of target cells with retroviral vectors. The retroviral vectors may comprise any transgene.

In a particular embodiment, the invention relates to the method according to the invention, wherein the vector comprises a p47phox, gp91phox, p22phox, p67phox or p40phox protein encoding cDNA in whole or in part.

That is, the retroviral vector may comprise a cDNA encoding any one of the proteins p47phox, gp91 phox, p22phox, p67phox or p40phox. In other embodiments, the retroviral vector may comprise fragments of cDNA encoding any one of the proteins p47phox, gp91phox, p22phox, p67phox or p40phox. The cDNA fragment may be a result of alternative splicing or may be generated by means of genetic engineering or chemical synthesis or may be any fusion construct comprising the cDNA encoding the proteins p47phox, gp91phox, p22phox, p67phox or p40phox. The fragment may comprise 50%, 60%, 70%, 80%, 90% or 95% of a cDNA encoding the proteins p47phox, gp91phox, p22phox, p67phox or p40phox. Preferably, the variant of p47phox, gp91phox, p22phox, p67phox or p40phox that is expressed from the cDNA fragment has the same biological function as the respective full length protein.

In a particular embodiment, the invention relates to the method according to the invention, wherein the vector comprises a transgene encoding a chimeric antigen receptor (CAR).

That is, the retroviral vector used in the method of the invention may comprise a nucleic acid encoding a CAR. The C is not limiting in the present method and may be any CAR known in the art.

In one embodiment of the invention, the transgene of interest, particularly p47phox, is under control of an internal promoter, particularly an internal promoter selected from the group consisting of the myelospecific miR223 promoter, simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters, but particularly the myelospecific miR223 promoter.

In one embodiment of the invention, the retroviral vector, particularly the lentiviral-SIN vector, comprising the p47phox transgene under control of a myelospecific promoter, particularly the miR223 promoter, is used in the method according to the present invention for the treatment of diseases or disorders associated with p47phox-deficiency, particularly for the treatment of p47phox-deficient form of chronic granulomatous disease. In certain embodiments, a lentiviral vector encoding a gp91phox or a p22phox or a p67phox or a p40phox encoding cDNA under control of the miR223 promoter is used in the method according to the present invention.

The term “miR223 promoter” refers to a DNA sequence of 250nts or more in length and a sequence homology of more than 70%, 75%, 80%, 85%, 90%, 95% to the sequence:

(SEQ ID NO: 1)
ACTTGTACAGCTTCACAGGGCTCCATGCTTAGAAGGACCCCACACTTAGT
TTAATGTTCTGCTGTCATCATCTTGATATTCTTAATTTTTAAATAAAGGG
CCTATCGTTTTCATTTTTTACTGGGCCTTGCAAATTATGTAGCTGGTTCT
GTATGCCAGGAGAGAAGTTGGAAGTAAAATGGTATTCCAGGACCAGGAGG
CATTCTGGCAGAGTGAAAGAACATGTGATTTGGAGTCCATGGGGATGGGT
TTAAATTTCAGCTTTCCACTAATTTGCTTTGTGATACTGAGTATTTCCTT
TTATCCCTCAGAGGCTCTGTTTCTCAATTTTGACTACGGGTTTTTCATTA
GATAATGTCTCAGTTCTGGTATTCCAGGTTTCCCTCAATTATTCTGGGAA
AACCTCCTTGACCCACAGGCAGAGCCTAGGGCAGCCAGGTGCTTTCTACT
CTCTCTCTCTCTGCAGCTTGGAAAGTTAGTGTCTGTTGAAGGTCAGCTGG
GAGTTGGTGGAGGCAGGGCAGTGGCCTGCTACTATTGCTGCAGTAGCAGA
CCCTTTCACAACAGCATTGTTTTGTCATTTTGCATCCAGATTTCCGTTGG
CTAACCTCAGTCTTATCTTCCTCATTTCTGTTTCCTGTTGAAGACACCAA
GGGCCCTTCAAAACACAGAAGCTTCTTGCTCACGGCAGAAAGCCCAATTC
CATCTGGCCCCTGCAGGTTGGCTCAGCACTGGGGAATCAGAGTCCCCTCC
ATGACCAAGGCACCACTCCACTGACAGGGATCCAAGCTTGCCACC

The term “p47phox protein” refers to any protein of 26 amino acids or more in length, comprising a sequence with a homology of more than 70%, 75%, 80%, 85%, 90%, 95% to any of the isoforms and/or splice variant encoded by human neutrophil cytosolic factor 1 (NCF-I) gene with NCBI GenelD 653361, and/or to any protein sequence with more than 70%, 75%, 80%, 85%, 90%, 95% homology to the protein sequence:

(SEQ ID NO: 2)

MGDTFIRHIA LLGFEKRFVP SQHYVYMFLV KWQDLSEKVV
YRRFTEIYEF HKTLKEMFPI EAGAINPENR IIPHLPAPKW
FDGQRAAENR QGTLTEYCST LMSLPTKISR CPHLLDFFKV
RPDDLKLPTD NQTKKPETYL MPKDGKSTAT DITGPIILQT
YRAIANYEKT SGSEMALSTG DVVEVVEKSE SGWWFCQMKA
KRGWIPASFL EPLDSPDETE DPEPNYAGEP YVAIKAYTAV
EGDEVSLLEG EAVEVIHKLL DGWWVIRKDD VTGYFPSMYL
QKSGQDVSQA QRQIKRGAPP RRSSIRNAHS IHQRSRKRLS
QDAYRRNSVR FLQQRRRQAR PGPQSPGSPL EEERQTQRSK
PQPAVPPRPS ADLILNRCSE STKRKLASAV

When a target cell is pre-stimulated and/or co-stimulated with a transduction enhancer or a combination of transduction enhancers, it is preferred that the target cell is incubated in the presence of the transduction enhancer or the combination of transduction enhancers in a liquid medium, preferably a liquid cell culture medium.

The skilled person is aware that the choice of cell culture medium depends on the type of target cell. That is, the cell culture medium is preferably a medium in which the target cell can be maintained and/or proliferated. A variety of cell culture media that are suitable for maintaining and/or proliferating cells of a specific cell types have been described in the art and are commercially available.

In certain embodiments, the target cell is a hematopoietic stern cell (HSC). Various media for cultivating HSC are known in the art. In certain embodiments, an HSC may be pre-stimulated and/or co-stimulated with a transduction enhancer or a combination of transduction enhancers by incubating the HSC in a liquid medium comprising X-Vivo 10 medium (Lonza), X-Vivo 20 medium (Lonza) or BESP1366F medium (modified X-VIVO 20 w/o antibiotics (gentamicin); Lonza).

In certain embodiment, the target cells, in particular HSC, may be incubated with a transduction enhancer or a combination of transduction enhancers in a cell culture medium, in particular an X-VIVO 10, X-VIVO 20 or BESP1366F medium, wherein the cell culture medium comprises 1% human serum albumin, 300 ng/ml stem cell factor (SCF), 200 ng/ml or 300 ng/ml fms like tyrosine kinase 3 (FLT-3) ligand (Flt3-lig) and/or 100 ng/ml thrombopoietin (TPO).

In certain embodiments, the target cells, in particular HSC, may be incubated with a transduction enhancer or a combination of transduction enhancers in X-VIVO 10 medium comprising 1% human serum albumin, 300 ng/ml stem cell factor (SCF), 300 ng/ml fins like tyrosine kinase 3 (FLT-3) ligand (Flt3-lig) and/or 100 ng/ml thrombopoietin (TPO).

In certain embodiments, the target cells, in particular HSC, may be incubated with a transduction enhancer or a combination of transduction enhancers in X-VIVO 20 medium comprising 1% human serum albumin, 300 ng/ml stem cell factor (SCF), 200 ng/ml fms like tyrosine kinase 3 (FLT-3) ligand (Flt3-lig) and/or 100 ng/ml thrombopoietin (TPO).

In certain embodiments, the target cells, in particular HSC, may be incubated with a transduction enhancer or a combination of transduction enhancers in BESP1366F medium comprising 1% human serum albumin, 300 ng/ml stem cell factor (SCF), 300 ng/ml fins like tyrosine kinase 3 (FLT-3) ligand (Flt3-lig) and/or 100 mg/ml thrombopoietin (TPO).

Target cells may be pre-stimulated and/or co-stimulated with a transduction enhancer or a combination of transduction enhancers at any cell density or concentration.

That is, target cells, in particular HSC, may be incubated with a transduction enhancer at a cell density ranging from about 1E3 to about 1E10 cells/cm², preferably at a cell density ranging from about 1E4 to about 1E8 cells/cm², more preferably at a cell density ranging from about 1E5 to about 1E7 cells/cm², most preferably at a cell density of about 2 E6 cells/cm².

Alternatively, target cells, in particular HSC, may be incubated with a transduction enhancer at a concentration ranging from about 1E3 to about 1E10 cells/mL, preferably at a concentration ranging from about 1E4 to about 1E8 cells/mL, more preferably at a concentration ranging from about 1E5 to about 1E7 cells/mL, most preferably at a concentration between 0.1E6 and 4 E6 cells/mL.

Several novel compounds and combination of compounds were tested for their potential to increase transduction efficiency of human cells by a gene therapy vector. Special focus was given to the combination of compounds with a retroviral vector, particularly a lentiviral-SIN vector, encoding a transgene of interest, such as, for example, but without limitation, p47phox.

In certain embodiments, the invention refers to the compound Amphotericin B for use as a transduction enhancer. That is, the present invention is based, at least in part, on the surprising finding that Amphotericin B can enhance the transduction efficiency of target cells with gene therapy vectors, in particular retroviral vectors. Accordingly, in a particular embodiment, the invention relates to the method according to the invention, wherein the transduction enhancer is Amphotericin B.

Amphotericin B is an antifungal medication used for the treatment of serious fungal infections and leishmaniosis. The fungal infections it is used to treat include aspergillosis, blastomycosis, candidiasis, coccidioidomycosis, and cryptococcosis. It is typically given by injection into a vein. Amphotericin B was isolated from Streptomyces nodosus in 1955 and came into medical use in 1958. It is on the World Health Organization's List of Essential Medicines, the safest and most effective medicines needed in a health system. Amphotericin B has not been suggested for use as a transduction enhancer.

It has been shown by the inventors that Amphotericin B enhances the transduction efficiency of a target cell when contacted with the target cell at a concentration of 0.5-1 μg/mL. Thus, in a certain embodiment, Amphotericin B may be used as a transduction enhancer at a concentration ranging from about 0.05 to about 10 μg/mL, preferably at a concentration ranging from about 0.1 to about 5 μg/mL, more preferably at a concentration ranging from about 0.5 to about 2 μg/mL, most preferably at a concentration of about 0.75 μg/mL. In certain embodiments, Amphotericin B may be used as a transduction enhancer at a concentration ranging from about 0.05 μM to about 500 μM, preferably at a concentration ranging from about 0.1 μM to about 10 μM, more preferably at a concentration of about 0.1 to about 3 μM, most preferably at a concentration of 0.811 μM. Preferably, Amphotericin B is contacted in the pre-stimulation or co-stimulation step with a hematopoietic cell, more preferably an HSC at any of the concentrations and/or densities disclosed above. In a certain embodiment, HSC at a concentration between 0.5 and 1 E6 cells/mL or at a concentration of 2 E6 cells/cm² cell culture surface may be pre-stimulated and/or co-stimulated with Amphotericin B at a concentration of 0.5 μg/mL In a certain embodiment, HSC at a concentration between 0.5 and 1 E6 cells/mL or at a concentration of 2E6 cells/cm² cell culture surface may be pre-stimulated and/or co-stimulated with Amphotericin B at a concentration of 0.75 μg/mL. In a certain embodiment, HSC at a concentration of between 0.5 and 1 E6 cells/mL or at a concentration of 2E6 cells/cm² cell culture surface may be pre-stimulated and/or co-stimulated with Amphotericin B at a concentration of 1 μg/mL.

In certain embodiments, the invention refers to the compound Silibinin for use as a transduction enhancer. That is, the present invention is based, at least in part, on the surprising finding that Silibinin can enhance the transduction efficiency of target cells with gene therapy vectors, in particular retroviral vectors. Accordingly, in a particular embodiment, the invention relates to the method according to the invention, wherein the transduction enhancer is Silibinin.

Silibinin, also known as silybin (both from Silybum, the generic name of the plant from which it is extracted), is the major active constituent of silymarin, a standardized extract of the milk thistle seeds, containing a mixture of flavonolignans consisting of silibinin, isosilibinin, silichristin, silidianin, and others. Silibinin itself is a mixture of two diastereomers, silybin A and silybin B, in approximately equimolar ratio. Commercially available Silibinin has the chemical name 2,3-Dihydro-3-(4-hydroxy-3-methoxyphenyl)-2-(hydroxymethyl)-6-(3,5,7-trihydroxy-4-oxobenzopyran-2-yl)benzodioxin (CAS Number: 22888-70-6).

Silibinin was reported to inhibit hepatitis B virus entry into HepG2-NTCP-C4 cells (Umetsu et al. (2018) Biochem Biophys Rep. 14: 20-25) and hepatitis C virus infection of primary human hepatocytes (Liu et al. (2017) Gut 66: 1853-1861). Silybin (SO), one major compound of S. marianum L., was reported to inhibit influenza A virus (IAV) infection of MDCK cells (Dai et al. (2013) Antimicrob Agents Chemother.57: 4433-43). Exposure of T cells during virus adsorption to Legalon-SIL (SIL), a water-soluble derivative of silibinin (but not silibinin), was reported to block HIV infection (McClure et al (2014) Virology 449:96-103).

No reports on Silibinin for use as a transduction enhancer exist. Further, in view of its anti-viral activities disclosed above, it is highly surprising that silibinin can be used to enhance the transduction of target cells with gene therapy vectors, in particular retroviral vectors.

It has been shown by the inventors that Silibinin enhances the transduction efficiency of a target cell when contacted with the target cell at a concentration of 1 to 5 μM. Thus, in a certain embodiment, Silibinin may be used as a transduction enhancer at a concentration ranging from about 0.05 to about 500 μM, preferably at a concentration ranging from about 0.05 to about 25 μM, more preferably at a concentration ranging from about 0.05 to about 10 μM, even more preferably at a concentration ranging from about 1 to about 10 μM, most preferably at a concentration of about 3 μM. Preferably, Silibinin is contacted in the pre-stimulation or co-stimulation step with a hematopoietic cell, more preferably an HSC at any of the concentrations and/or densities disclosed above. In a certain embodiment, HSC at a concentration of 0.5 to 1 E6 cells/mL or in a density of 2E6/cm² may be pre-stimulated and/or co-stimulated with Silibinin at a concentration of 3 μM. In a certain embodiment, HSC at a concentration of 0.5 to 1 E6 cells/mL or in a density of 2E6/cm² may be pre-stimulated and/or co-stimulated with Silibinin at a concentration of 5 μM.

In certain embodiments, the invention refers to the compound Midostaurin for use as a transduction enhancer. That is, the present invention is based, at least in part, on the surprising finding that Midostaurin can enhance the transduction efficiency of target cells with gene therapy vectors, in particular retroviral vectors. Accordingly, in a particular embodiment, the invention relates to the method according to the invention, wherein the transduction enhancer is Midostaurin.

The protein kinase inhibitor Midostaurin, also known as CGP 41251, was initially developed as anticancer drug (Meyer et al. (1989) Int J Cancer 43: 851-6). The compound was reported to reactivate HIV-1 expression from the HIV-1 latently infected ACH2 cell line, and from primary resting CD4+ T cells (Ao et al. (2016) Virol J 13: 177). The drug has never been tested for its potency to increase transduction efficiency.

It has been shown by the inventors that Midostaurin enhances the transduction efficiency of a target cell when contacted with the target cell at a concentration of 100 to 400 nM. Thus, in a certain embodiment, Midostaurin may be used as a transduction enhancer at a concentration ranging from about 50 to about 500,000 nM, preferably at a concentration ranging from about 50 to about 25,000 nM, more preferably at a concentration ranging from about 50 to about 10000 nM, even more preferably at a concentration ranging from about 50 to about 5000 nM, even more preferably at a concentration ranging from about 50 to about 1000 nM, even more preferably at a concentration ranging from about 50 to about 500 nM, most preferably at a concentration of about 200 nM. Preferably, Midostaurin is contacted in the pre-stimulation or co-stimulation step with a hematopoietic cell, more preferably an HSC at any of the concentrations and/or densities disclosed above. In a certain embodiment, HSC at a concentration of 0.5 E6 cells/mL or in a density of 2E6/cm² may be pre-stimulated and/or co-stimulated with Midostaurin at a concentration of 100 nM. In a certain embodiment, HSC at a concentration of 0.5 E6 to 1 E6 cells/mL or in a density of 2E6/cm² may be pre-stimulated and/or co-stimulated with Midostaurin at a concentration of 200 nM. In a certain embodiment, HSC at a concentration of 0.5 E6 to 1 E6 cells/mL or in a density of 2E6/cm² may be pre-stimulated and/or co-stimulated with Midostaurin at a concentration of 400 nM.

In certain embodiments, the invention refers to the compound Nystatin for use as a transduction enhancer. That is, the present invention is based, at least in part, on the surprising finding that Nystatin can enhance the transduction efficiency of target cells with gene therapy vectors, in particular retroviral vectors. Accordingly, in a particular embodiment, the invention relates to the method according to the invention, wherein the transduction enhancer is Nystatin.

Nystatin is generally used in cell culture for its antimycotic action (Fassler et al. (2013) PLoS One 8:e76092). Nystatin is also a cholesterol-binding reagent, known to disrupt caveolin-mediated endocytosis. It was tested in the past for its ability to inhibit the transduction of 293T cells with wildtype lentiviral vectors. No inhibition of vector entry by nystatin was observed, confirming that wildtype lentiviral vectors use clathrin-mediated endocytosis to enter the cells (Lee, Dang, Joo & Wang (2011) Virus Res. 160: 340-50). Treatment of lentiviral particles with Nystatin, followed by repurification of lentiviral particles from nystatin, led to a significant decrease in infectivity of re-purified lentiviral particles (Guyader et al. (2002) J Virol. 76: 10356-64). A transduction enhancing effect of Nystatin on retroviral transduction was never reported up to now.

It has been shown by the inventors that Nystatin enhances the transduction efficiency of a target cell when contacted with the target cell at a concentration of 100 μM. Thus, in a certain embodiment, Nystatin may be used as a transduction enhancer at a concentration ranging from about 10 to about 1000 μM, preferably at a concentration ranging from about 25 to about 500 μM, more preferably at a concentration ranging from about 50 to about 250 μM, most preferably at a concentration of about 100 μM. Preferably, Nystatin is contacted in the pre-stimulation or co-stimulation step with a hematopoietic cell, more preferably an HSC at any of the concentrations and/or densities disclosed above. In a certain embodiment, HSC at a concentration of 0.5 to 1 E6 cells/mL or in a density of 2E6/cm² may be pre-stimulated and/or co-stimulated with Nystatin at a concentration of 100 μM.

In certain embodiments, the invention refers to the compound Natamycin for use as a transduction enhancer. That is, the present invention is based, at least in part, on the surprising finding that Natamycin can enhance the transduction efficiency of target cells with gene therapy vectors, in particular retroviral vectors. Accordingly, in a particular embodiment, the invention relates to the method according to the invention, wherein the transduction enhancer is Natamycin.

Natamycin is an antifungal agent used in the food industry for the surface treatment of sausages and cheese (Juneja, Dwivedi & Yan (2012) Annu Rev Food Sci Technol. 3:381-403). It was never reported in the context of viral transduction.

It has been shown by the inventors that Natamycin enhances the transduction efficiency of a target cell when contacted with the target cell at a concentration of 3 μM. Thus, in a certain embodiment, Natamycin may be used as a transduction enhancer at a concentration ranging from about 0.05 to about 500 μM, preferably at a concentration ranging from about 0.05 to about 10 μM, more preferably at a concentration ranging from about 1 to about 5 μM, most preferably at a concentration of about 3 μM. In a certain embodiment, Natamycin may be used as a transduction enhancer at a concentration ranging from about 0.1 to about 20 μM. Preferably, Natamycin is contacted in the pre-stimulation or co-stimulation step with a hematopoietic cell, more preferably an HSC at any of the concentrations and/or densities disclosed above. In a certain embodiment, HSC at a concentration of 0.5 to 1 E6 cells/mL or in a density of 2E6/cm² may be pre-stimulated and/or co-stimulated with Natamycin at a concentration of about 3 μM.

In certain embodiments, the invention refers to the compound Everolimus for use as a transduction enhancer. That is, the present invention is based, at least in part, on the surprising finding that Everolimus can enhance the transduction efficiency of target cells with gene therapy vectors, in particular retroviral vectors. Accordingly, in a particular embodiment, the invention relates to the method according to the invention, wherein the transduction enhancer is Everolimus.

Everolimus is a medication used as an immunosuppressant to prevent rejection of organ transplants and in the treatment of renal cell cancer and other tumors. Much research has also been conducted on everolimus and other mTOR inhibitors as targeted therapy for use in a number of cancers.

It has been shown by the inventors that Everolimus enhances the transduction efficiency of a target cell when contacted with the target cell at a concentration of 1 μM. Thus, in a certain embodiment, Everolimus may be used as a transduction enhancer at a concentration ranging from about 0.1 to about 10 μM, preferably at a concentration ranging from about 0.2 to about 7.5 μM, more preferably at a concentration ranging from about 0.5 to about 5 μM, most preferably at a concentration of about 1 μM. Preferably, Everolimus is contacted in the pre-stimulation or co-stimulation step with a hematopoietic cell, more preferably an HSC at any of the concentrations and/or densities disclosed above. In a certain embodiment, HSC at a concentration of 0.5 to 1 E6 cells/mL or in a density of 2E6/cm² may be pre-stimulated and/or co-stimulated with Everolimus at a concentration of 1 μM.

In certain embodiments, the invention refers to the compound deoxyribonucleosides for use as a transduction enhancer. That is, the present invention is based, at least in part, on the surprising finding that deoxyribonucleosides can enhance the transduction efficiency of target cells with gene therapy vectors, in particular retroviral vectors. Accordingly, in a particular embodiment, the invention relates to the method according to the invention, wherein the transduction enhancer is deoxyribonucleosides.

The term “deoxyribonucleosides” comprises any composition of 2′-Deoxythymidine, i.e. the chemical structure with CAS number 50-89-5 with the synonyms Thymine deoxyriboside, 1-(2-Deoxy-β-D-ribofuranosyl)-5-methyluracil, 1-(2-Deoxy-β-D-ribofuranosyl)thymine, dT), and 2′-Deoxyadenosine, i.e. the chemical structure with CAS number 958-09-8 with the synonyms 9-(2- Deoxy-β-D-ribofuranosyl)adenine, Adenine deoxyriboside), and 2′-Deoxyguanosine, i.e. the chemical structure with CAS number 312693-72-4 with the synonyms 9-(2-Deoxy-β-D-ribofuranosyl)guanine, Guanine-2′-deoxyriboside) and 2′-Deoxycytidine (i.e. the chemical structure with CAS number 951-77-9 and synonym Cytosine deoxyriboside). Each deoxyribonucleoside may be present in the same concentration or at different concentrations. In a preferred embodiment, all four deoxyribonucleosides listed above are present in equimolar amounts. Deoxyribonucleosides have not been suggested as a transduction enhancer for haematopoietic stem cells.

It has been shown by the inventors that deoxyribonucleosides have the ability to enhance the transduction efficiency of haematopoietic stem cells as target cell when contacted with the target cell at a final concentration of 0.3-2.5 mM. Thus, in a certain embodiment, deoxyribonucleosides may be used as a transduction enhancer at a concentration ranging from about 0.1 to about 10 mM, preferably at a concentration ranging from about 0.25 to about 7.5 mM, more preferably at a concentration ranging from about 0.5 to about 5 mM, most preferably at a concentration of about 2.5 mM. Preferably, deoxyribonucleosides is contacted in the pre-stimulation or co-stimulation step with a hematopoietic cell, more preferably an HSC at any of the concentrations and/or densities disclosed above. In a certain embodiment, HSC at a concentration of 0.5 to 1 E6 cells/mL or in a density of 2E6/cm² may be pre-stimulated and/or co-stimulated with deoxyribonucleosides at a concentration of 0.3 mM.

In certain embodiments, the invention refers to the use of BAB-type triblock copolymers as a transduction enhancer. That is, the present invention is based, at least in part, on the surprising finding that BAB-type triblock copolymers can enhance the transduction efficiency of target cells with gene therapy vectors, in particular retroviral vectors. Accordingly, in a particular embodiment, the invention relates to the method according to the invention, wherein the transduction enhancer is a BAB-type triblock copolymer.

The term “BAB-type triblock copolymer” refers to any polymer consisting of a linear arrangement of three blocks, with each block consisting of a polymeric form of repetitive elements, in which the hydrophobic polymeric block “A” in the center is flanked on both sides by hydrophilic polymeric units on both sides, referred as “B”. Polymers here referred as “BAB-type triblock copolymer” are synthesized by covalent linkage of two “BA” di-block copolymers by a linker with the linker referred as “L”, wherein “L” is preferably, but not exclusively, hexamethylene diisocyanate (HMDI). In “BAB-type triblock copolymers”, the peripheral block “B” may be formed preferably, but not exclusively, by polymers of ethylene glycol with the formula —(CH₂—CH₂—O)ₓ—, and the “A” block may comprise or consist of poly(D,L-lactic acid-co-glycolic acid) (PLGA) of poly(lactide) (PLA) or of poly(-caprolacton) (PCL). The corresponding “BAB-type triblock copolymers” are referred to as “PEG-PLGA-PEG”, “PEG-PLA-PEG” and “PEG-PCL-PEG” polymers, respectively.

The term “PEG-PLGA-PEG” polymer refers to a BAB-type triblock copolymer, with the “B” block formed by a polymer of ethylene glycol with the formula of —(CH₂—CH₂—O)ₓ—, and the “A” block comprising or consisting of poly(D,L-lactic acid-co-glycolic acid). The resulting polymer is termed “methoxy poly(ethylene glycol)-b-poly(D,L-lactic acid-co-glycolic acid)-b-methoxy poly(ethylene glycol)” (“mPEG-PLGA-mPEG”) and may be summarized by the formula CH₃—O—(CH₂—CH₂—O)ₓ—(CO—CH₂—O)_{y}—(CO—CHCH₃—O)z-L-(O—CHCH₃—CO)z—(O—CH₂—CO)y—(O—CH₂—CH₂)ₓ—O—CH₃ (with L=CO—NH—CH₂—(CH₂)₄—CH₂—NH—CO in case of HMDI linker, and x,y,z the number of monomers within the polymer). The same molecule may also be termed poly(ethylene glycol)-b-poly(D,L-lactic acid-co-glycolic acid)-b-poly(ethylene glycol) (“PEG-PLGA-PEG”) and may be summarized by the formula CH₃—(O—CH₂—CH₂)ₓ—(O—CO—CH₂)_{y}—(O—CO—CHCH₃)_{z}—O-L-O—(CHCH₃—CO—O)_{z}—(CH₂—CO—O)_{y}—(CH₂—CH₂—O)ₓ—CH₃ (with L=CO—NH—CH2-(CH2)₄—CH2-NH—CO in ease of HMDI linker, and x,y,z the number of monomers within the polymer).

In polymers herein referred to as “PEG-PLGA-PEG” polymer, “L” comprises or consists preferably, but not exclusively of —CO—NH—CH2-(CH2)₄-CH2-NH—CO— in case of HMDI linkers. Polymers termed “PEG-PLGA-PEG” herein may have a molecular weight between 10,000 and 16,000 Dalton, preferably of about 10,000 Dalton, of about 11,000 Dalton, of about 12,000 Dalton, of about 13,000 Dalton, of about 14,000 Dalton, of about 15,000 Dalton of about 16,000 Dalton. The “B” block may consist of polymers preferably, but not exclusively, formed by polymers of ethylene glycol. The PEG portion of the polymer may contribute to the total molecular weight of the polymer by more than 50% and less than 95%. That is, about 50%, about 55%, about 60%, about 65%, about 70%, about 75%, about 80%, about 85%, about 90% or about 95% of the total molecular weight of the polymer “PEG-PLGA-PEG” may be attributed to PEG polymers. In certain embodiments, PEG-PLGA-PEG may refer to poly(ethylene glycol)-b-poly(D,L-lactic acid-co-glycolic acid)-b-poly(ethylene glycol) (PEG- PLGA-PEG) with 5 kDa poly(ethylene glycol) blocks on both ends, and a central 4.2 kDa poly(D,L-lactic acid-co-glycolic acid) block, termed PEG5k-b-PLGA4.2k-b-PEG5k.

The PEG-PLGA-PEG polymer was described to form micelles in a concentration and temperature dependent manner (Jeong, Bae & Kim (1999) Colloids and Surfaces B: Biointerfaces 16: 185-93), and may be used for drug delivery (Tyagi et al. (2004) Pharm Res. 21: 832-7). The above mentioned PEG-PLGA-PEG polymers were never reported for viral transduction enhancing activity.

PEG-PLGA-PEG may be used as a transduction enhancer at a concentration ranging from about 20 μg/ml to about 5000 μg/ml, preferably at a concentration ranging from about 100 μg/ml to about 3500 μg/ml, more preferably at a concentration ranging from about 500 ng/ml to about 2000 μg/ml, most preferably at a concentration of about 1000 μg/ml. Preferably, PEG-PLGA-PEG is contacted in the pre-stimulation or co-stimulation step with a hematopoietic cell, more preferably an HSC at any of the concentrations and/or densities disclosed above. In a certain embodiment, HSC at a concentration of 0.5 to 1 E6 cells/mL or in a density of 2E6/cm² may be pre-stimulated and/or co-stimulated with PEG-PLGA-PEG at a concentration of 1000 μg/ml.

The term “PEG-PLA-PEG” polymer refers to a BAB-type triblock copolymer with the “B” block formed by polymer of ethylene glycol with the formula of —(CH₂—CH₂—O)ₙ—, and the “A” block comprising or consisting of a polymeric form of lactic acid. Polymeric forms of lactic acid comprise polymeric forms of enantiomeric L- and/or D-lactic acids also known as poly(L-lactide) (PLLA) and poly(D-lactide) (PDLA). The resulting polymers may be termed methoxypoly(ethylene glycol)/poly(lactide)/methoxypoly(ethylene glycol) (PEG-PLA-PEG or mPEG-PLA-mPEG, herein collectively referred as PEG-PLA-PEG) and summarized by the formula CH₃—O—(CH₂—CH₂—O)ₙ—(CO—CCH₃—O)ₘ—L-(O—CCH3-CO)ₘ—(O—CH₂—CH₂)ₙ—O—CH₃ (with L=CO—NH—CH2-(CH2)₄-CH2—NH—CO in case of HMDI linker, and n, m the number of monomers within the polymer).

In polymers herein referred to as “PEG-PLA-PEG” polymer, “L” comprises or consists preferably, but not exclusively, of CO—NH—CH2-(CH2)₄-CH2-NH—CO in case of HMDI linkers. Polymers herein referred to as “PEG-PLA-PEG” polymer may have a molecular weight between 10,000 and 16,000 Dalton, preferably of about 10,000 Dalton, of about 11,000 Dalton, of about 12,000 Dalton, of about 13,000 Dalton, of about 14,000 Dalton, of about 15,000 Dalton of about 16,000 Dalton. The “B” block may consist of polymers preferably, but not exclusively, formed by polymers of ethylene glycol. The PEG portion of the polymer may contribute to the total molecular weight of the polymer by more than 50% and less than 95%. That is, about 50%, about 55%, about 60%, about 65%, about 70%, about 75%, about 80%, about 85%, about 90% or about 95% of the total molecular weight of the polymer “PEG-PLA-PEG” may be attributed to PEG polymers. In certain embodiments, PEG-PLA-PEG may refer to poly(ethylene glycol)/poly(lactide)/poly(ethylene glycol) (PEG-PLA-PEG) with 5 kDa poly(ethylene glycol) blocks on both ends, and a central 4.2 kDa poly(lactide) block, termed PEG5k-b-PLA4.2k-b-PEG5k.

It has been shown by the inventors that PEG-PLA-PEG enhances the transduction efficiency of a target cell when contacted with the target cell at a concentration of 10 μg/mL, Thus, in a certain embodiment, PEG-PLA-PEG may be used as a transduction enhancer at a concentration ranging from about 0.1 μg/ml to about 5000 μg/ml, preferably at a concentration ranging from about 1 μg/ml to about 2500 μg/ml, more preferably at a concentration ranging from about 5 μg/ml to about 1000 μg/ml, most preferably at a concentration of about 50 μg/ml. In certain embodiments, PEG-PLA-PEG may be used as a transduction enhancer at a concentration ranging from about 1 μg/ml to about 100 μg/ml, preferably at a concentration ranging from about 5 μg/ml to about 50 μg/ml. Preferably, PEG-PLA-PEG is contacted in the pre-stimulation or co-stimulation step with a hematopoietic cell, more preferably an HSC at any of the concentrations and/or densities disclosed above. In a certain embodiment, HSC at a concentration of 0.5 to 1 E6 cells/mL or in a density of 2E6/cm² may be pre-stimulated and/or co-stimulated with PEG-PLA-PEG at a concentration of 50 μg/ml.

The term “PEG-PCL-PEG” polymer refers to a BAB-type triblock copolymer with the “B” block formed by polymer of ethylene glycol with the formula of —(CH₂—CH₂—O)ₙ—, and the “A” block comprising or consisting of a polymeric form of e-caprolacton. The resulting polymer may be termed “methoxy poly(ethylene glycol)-poly(e-caprolacton)-methoxypoly(ethylene glycol) (PEG-PCL-PEG) and may be summarized by the formula CH₃—O—(CH₂—CH₂—O)ₙ—(CO—CH₂—CH₂—CH₂—CH₂—CH₂—O)ₘ-L-(O—CCH₃—CO)ₘ—(O—CH₂—CH₂)ₙ—O—CH₃ (with L=CO—NH—CH2-(CH2)₄—CH2-NH—CO in case of HMDI linker, and n, m the number of monomers within the polymer).

In polymers herein referred to as “PEG-PCL-PEG” polymers, “L” may comprise or consist preferably, but not exclusively, of L=CO—NH—CH2-(CH2)₄—CH2-NH—CO in case of HMDI linkers. Polymers herein referred to as “PEG-PCL-PEG” polymers may have a molecular weight between 10,000 and 16,000 Dalton, preferably of about 10,000 Dalton, of about 11,000 Dalton, of about 12,000 Dalton, of about 13,000 Dalton, of about 14,000 Dalton, of about 15,000 Dalton of about 16,000 Dalton. The “B” block may consist of polymers preferably, but not exclusively, formed by polymers of ethylene glycol. The PEG portion of the polymer may contribute to the total molecular weight of the polymer by more than 50% and less than 95%. That is, about 50%, about 55%, about 60%, about 65%, about 70%, about 75%, about 80%, about 85%, about 90% or about 95% of the total molecular weight of the polymer “PEG-PCL-PEG” may be attributed to PEG polymers. In certain embodiments, PEG-PCL-PEG may refer to poly(ethylene glycol)-poly(e-caprolacton)-poly(ethylene glycol) (PEG-PCL-PEG) with 5 kDa poly(ethylene glycol) blocks on both ends, and a central 4.2 kDa poly(e-caprolacton) block, termed PEG5k-b-PCL4.2k-b-PEG5k. In certain embodiments, PEG-PCL-PEG may refer to poly(ethylene glycol)-poly(e-caprolacton)-poly(ethylene glycol) (PEG-PCL-PEG) with 5.3 kDa poly(ethylene glycol) blocks on both ends, and a central 2.4 kDa poly(e-caprolacton) block, termed NH2-PEG5.3k-b- PCL2.4k-b-PEG5.3k-NH2.

It has been shown by the inventors that PEG-PCL-PEG enhances the transduction efficiency of a target cell when contacted with the target cell at a concentration of 10 μg/mL. Thus, in a certain embodiment, PEG-PCL-PEG may be used as a transduction enhancer at a concentration ranging from about 0.1 μg/ml to about 5000 μg/ml, preferably at a concentration ranging from about 1 μg/ml to about 2500 μg/ml, more preferably at a concentration ranging from about 5 μg/ml to about 1000 μg/ml, most preferably at a concentration of about 10 μg/ml. In certain embodiments, PEG-PCL-PEG may be used as a transduction enhancer at a concentration ranging from about 1 μg/ml to about 100 μg/ml, preferably at a concentration ranging from about 5 μg/ml to about 50 μg/ml. Preferably, PEG-PCL-PEG is contacted in the pre-stimulation or co-stimulation step with a hematopoietic cell, more preferably an HSC at any of the concentrations and/or densities disclosed above. In a certain embodiment, HSC at a concentration of 0.5 to 1 E6 cells/mL or in a density of 2E6/cm² may be pre-stimulated and/or co-stimulated with PEG-PCL-PEG at a concentration of 10 μg/ml.

The present invention further encompasses the use of functionalized BAB-type triblock-polymers for use as a transduction enhancer. The term “functionalized” polymer refers to a “BAB-type triblock copolymer”, including “PEG-PLGA-PEG” polymers, “PEG-PLA-PEG” polymers and “PEG-PCL-PEG” polymers, which were “functionalized” by covalent linkage of a cationic group to one and/or both ends of the polymer. In these functionalized “BAB-type triblock copolymers”, the cationic groups may comprise or consist of molecules comprising amino groups, such as, without limitation, monomeric and/or polymeric forms of lysine, arginine and/or histidine.

In certain embodiments, the invention refers to the compound Resveratrol for use as a transduction enhancer. That is, the present invention is based, at least in part, on the surprising finding that Resveratrol can enhance the transduction efficiency of target cells with gene therapy vectors, in particular retroviral vectors. Accordingly, in a particular embodiment, the invention relates to the method according to the invention, wherein the transduction enhancer is Resveratrol.

Resveratrol (3,5,4′-trihydroxy-trans-stilbene) is a stilbenoid, a type of natural phenol, and a phytoalexin produced by several plants in response to injury or when the plant is under attack by pathogens, such as bacteria or fungi. Resveratrol has been studied for its potential therapeutic use, with little evidence of anti-disease effects or health benefits in humans.

It has been shown by the inventors that Resveratrol enhances the transduction efficiency of a target cell when contacted with the target cell at a concentration of 5 μM. Thus, in a certain embodiment, Resveratrol may be used as a transduction enhancer at a concentration ranging from about 0.1 to about 10 μM, preferably at a concentration ranging from about 1 to about 7.5 μM, more preferably at a concentration ranging from about 2.5 to about 7.5 μM, most preferably at a concentration of about 5 μM. Preferably, Resveratrol is contacted in the pre-stimulation or co-stimulation step with a hematopoietic cell, more preferably an HSC at any of the concentrations and/or densities disclosed above. In a certain embodiment, HSC at a concentration of 0.5 to 1 E6 cells/mL or in a density of 2E6/cm² may be pre-stimulated and/or co-stimulated with Resveratrol at a concentration of 5 μM.

In certain embodiments, the invention refers to the compound Prostaglandin E2 for use as a transduction enhancer. That is, the present invention is based, at least in part, on the surprising finding that Prostaglandin E2 can enhance the transduction efficiency of target cells with gene therapy vectors, in particular retroviral vectors. Accordingly, in a particular embodiment, the invention relates to the method according to the invention, wherein the transduction enhancer is Prostaglandin E2.

Prostaglandin E2 (PGE2), also known as dinoprostone, is a naturally occurring prostaglandin with oxytocic properties that is used as a medication. Dinoprostone is used in labor induction, bleeding after delivery, termination of pregnancy, and in newborn babies to keep the ductus arteriosus open. In babies it is used in those with congenital heart defects until surgery can be carried out. It is also used to manage gestational trophoblastic disease.

It has been shown by the inventors that Prostaglandin E2 enhances the transduction efficiency of a target cell when contacted with the target cell at a concentration of 10 μM. Thus, in a certain embodiment, Prostaglandin E2 may be used as a transduction enhancer at a concentration ranging from about 1 to about 100 μM, preferably at a concentration ranging from about 2 to about 50 μM, more preferably at a concentration ranging from about 5 to about 25 μM, most preferably at a concentration of about 10 μM. Preferably, Prostaglandin E2 is contacted in the pre-stimulation or co-stimulation step with a hematopoietic cell, more preferably an HSC at any of the concentrations and/or densities disclosed above. In a certain embodiment, HSC at a concentration of 0.5 to 1 E6 cells/mL or in a density of 2E6/cm² may be pre-stimulated and/or co-stimulated with Prostaglandin E2 at a concentration of 10 μM.

In certain embodiments, the invention refers to the compound Poloxamer synperonic F108 for use as a transduction enhancer. That is, the present invention is based, at least in part, on the surprising finding that Poloxamer synperonic F108 can enhance the transduction efficiency of target cells with gene therapy vectors, in particular retroviral vectors. Accordingly, in a particular embodiment, the invention relates to the method according to the invention, wherein the transduction enhancer is Poloxamer synperonic F108.

Poloxamer synperonic F108 is a non-ionic polymeric surfactant.

It has been shown by the inventors that Poloxamer synperonic F108 enhances the transduction efficiency of a target cell when contacted with the target cell at a concentration of 0.5-2 mg/mL. Thus, in a certain embodiment, Poloxamer synperonic F108 may be used as a transduction enhancer at a concentration ranging from about 0.1 to about 10 mg/mL, preferably at a concentration ranging from about 0.25 to about 5 mg/mL, more preferably at a concentration ranging from about 0.5 to about 2 mg/mL, most preferably at a concentration of about 1 mg/mL. Preferably, Poloxamer synperonic F108 is contacted in the pre-stimulation or co-stimulation step with a hematopoietic cell, more preferably an HSC at any of the concentrations and/or densities disclosed above. In a certain embodiment, HSC at a concentration of 0.5 E6 cells/mL may be pre-stimulated and/or co-stimulated with Poloxamer synperonic F108 at a concentration of 0.5 mg/mL. In a certain embodiment, HSC at a concentration of 0.5 to 1 E6 cells/mL or in a density of 2E6/cm² may be pre-stimulated and/or co-stimulated with Poloxamer synperonic F108 at a concentration of 1 mg/mL. In a certain embodiment, HSC at a concentration of 0.5 to 1 E6 cells/mL or in a density of 2E6/cm² may be pre-stimulated and/or co-stimulated with Poloxamer synperonic F108 at a concentration of 2 mg/mL.

In certain embodiments, the invention refers to the compound Dimethyl sulfoxide (DMSO) for use as a transduction enhancer. That is, the present invention is based, at least in part, on the surprising finding that DMSO can enhance the transduction efficiency of target cells with gene therapy vectors, in particular retroviral vectors. Accordingly, in a particular embodiment, the invention relates to the method according to the invention, wherein the transduction enhancer is DMSO.

Dimethyl sulfoxide (DMSO) is an organosulfur compound with the formula (CH₃)₂SO. This colorless liquid is an important polar aprotic solvent that dissolves both polar and nonpolar compounds and is miscible in a wide range of organic solvents as well as water.

It has been shown by the inventors that DMSO enhances the transduction efficiency of a target cell when contacted with the target cell at a concentration of 1% (v/v). Thus, in a certain embodiment, DMSO may be used as a transduction enhancer at a concentration ranging from about 0.1 to about 10% (v/v), preferably at a concentration ranging from about 0.25 to about 5% (v/v), more preferably at a concentration ranging from about 0.5 to about 2% (v/v), most preferably at a concentration of about 1% (v/v). Preferably, DMSO is contacted in the pre-stimulation or co-stimulation step with a hematopoietic cell, more preferably an HSC at any of the concentrations and/or densities disclosed above. In a certain embodiment, HSC at a concentration of 0.5 E6 cells/mL may be pre-stimulated and/or co-stimulated with DMSO at a concentration of 0.5% (v/v). In a certain embodiment, HSC at a concentration of 0.5 to 1 E6 cells/mL or in a density of 2E6/cm² may be pre-stimulated and/or co-stimulated with DMSO at a concentration of 1% (v/v). In a certain embodiment, HSC at a concentration of 0.5 to 1 E6 cells/mL or in a density of 2E6/cm² may be pre-stimulated and/or co-stimulated with DMSO at a concentration of 2% (v/v).

It has been surprisingly shown by the inventors that certain combinations of the compounds disclosed herein may result in enhanced transduction efficiencies and have to be considered relevant for the future applications of multiple transduction enhancers in all these procedures.

Furthermore, the commercially available compound Lentiboost® has been described to enhance the transduction of various human target cells with lentiviral vectors. The inventors have surprisingly shown that the transduction efficiency of Lentiboost® can be further increased by combining Lentiboost® with an additional transduction enhancer. Lentiboost consists of a combination of Poloxamer F108 and polybrene, however, the exact ratios of the two compounds has not been disclosed in the art. An exemplary mixture of the two compounds is disclosed in Example 2.

That is, in certain embodiments, the invention relates to the method according to the invention, wherein the transduction enhancer is a combination of Lentiboost® and

Amphotericin B.

Lentiboost® is recommended to be used at a concentration of 1 mg/mL. Transduction of HSCs with a lentiviral vector at an MOI of 10 in the presence of 1 mg/mL Lentiboost® results in a vector copy number (VCN) of approximately 5. It has been shown by the inventors that the combination of 1 mg/mL of Lentiboost® with 0.5 to 1 μg/mL of Amphotericin B results in VCNs ranging from 8 to 10 under comparable conditions. Interestingly, simply increasing the concentration of Lentiboost® to 2 mg/mL only resulted in a VCN of approximately 6. Thus, the inventors have surprisingly shown that the combination of Lentiboost® and Amphotericin B results in increased transduction efficiencies.

Lentiboost® may be used in combination with Amphotericin B as a transduction enhancer at any suitable concentration. In certain embodiments, the combination of Lentiboost® and Amphotericin B may comprise Lentiboost® at a concentration ranging from about 0.1 mg/mL to about 10 mg/mL and Amphotericin B at a concentration ranging from about 0.1 μg/mL to about 10 μg/mL. In certain embodiments, the combination of Lentiboost® and Amphotericin B may comprise Lentiboost® at a concentration ranging from about 0.1 mg/mL to about 10 mg/mL and Amphotericin B at a concentration ranging from about 0.1 μg/mL to about 10 μg/mL. In certain embodiments, Lentiboost® may be added to a target cell at a final concentration ranging from about 0.1 mg/mL to about 10 mg/mL in combination with Amphotericin B at a final concentration ranging from about 0.1 μg/mL to about 10 μg/mL. Preferably, Lentiboost® may be added to a target cell at a final concentration ranging from about 0.1 mg/mL to about 3 mg/mL in combination with Amphotericin B at a final concentration ranging from about 0.1 μg/mL to about 3 μg/mL. More preferably, Lentiboost® may be added to a target cell at a final concentration ranging from about 0.5 mg/mL to about 2 mg/mL in combination with Amphotericin B at a final concentration ranging from about 0.5 μg/mL to about 2 μg/mL. Most preferably, Lentiboost® may be added to a target cell at a final concentration of about 1 mg/mL in combination with Amphotericin B at a final concentration of about 0.75 μg/mL. Preferably, the combination of Lentiboost® and Amphotericin B is contacted in the pre-stimulation or co-stimulation step with a hematopoietic cell, more preferably an HSC at any of the concentrations and/or densities disclosed above. In a certain embodiment, HSC at a concentration of 0.5 to 1 E6 cells/mL or at a density of 2E6/cm² may be pre-stimulated and/or co-stimulated with a combination of 1 mg/mL Lentiboost® and 0.5 μg/mL of Amphotericin B. In a certain embodiment, HSC at a concentration of 0.5 to 1 E6 cells/mL or at a density of 2E6/cm² may be pre-stimulated and/or co-stimulated with a combination of 1 mg/mL Lentiboost® and 0.75 μg/mL of Amphotericin B. In a certain embodiment, HSC at a concentration of 0.5 to 1 E6 cells/mL or at a density of 2E6/cm² may be pre-stimulated and/or co-stimulated with a combination of 1 mg/mL Lentiboost® and 1 μg/mL of Amphotericin B.

In certain embodiments, the invention relates to the method according to the invention, wherein the transduction enhancer is a combination of Lentiboost® and Silibinin.

It has been shown by the inventors that Lentiboost®, when used in combination with Silibinin, results in a higher transduction efficiency compared to Lentiboost® alone. Lentiboost® may be used in combination with Silibinin as a transduction enhancer at any suitable concentration. In certain embodiments, the combination of Lentiboost® and Silibinin may comprise Lentiboost® at a concentration ranging from about 0.1 mg/mL to about 10 mg/mL and Silibinin at a concentration ranging from about 0.1 to about 25 μM. In certain embodiments, Lentiboost® may be added to a target cell at a final concentration ranging from about 0.1 μg/mL to about 10 mg/mL in combination with Silibinin at a final concentration ranging from about 0.1 to about 25 μM. Preferably, Lentiboost® may be added to a target cell at a final concentration ranging from about 0.1 mg/mL to about 3 mg/mL in combination with Silibinin at a final concentration ranging from about 0.1 to about 10 μM. More preferably, Lentiboost® may be added to a target cell at a final concentration ranging from about 0.5 mg/mL to about 2 mg/mL in combination with Silibinin at a final concentration ranging from about 1 to about 10 μM. Most preferably, Lentiboost® may be added to a target cell at a final concentration of about 1 mg/mL in combination with Silibinin at a final concentration of about 5 μM. Preferably, the combination of Lentiboost® and Silibinin is contacted in the pre-stimulation or co-stimulation step with a hematopoietic cell, more preferably an HSC at any of the concentrations and/or densities disclosed above. In a certain embodiment, HSC at a concentration of 0.5 to 1 E6 cells/mL or at a density of 2E6/cm² may be pre-stimulated and/or co-stimulated with a combination of 1 mg/mL Lentiboost and 1 μM of Silibinin. In a certain embodiment, HSC at a concentration of 0.5 to 1 E6 cells/mL or at a density of 2E6/cm² may be pre-stimulated and/or co-stimulated with a combination of 1 mg/mL Lentiboost and 5 μM of Silibinin.

In certain embodiments, the invention relates to the method according to the invention, wherein the transduction enhancer is a combination of Lentiboost® and Midostaurin.

It has been shown by the inventors that Lentiboost®, when used in combination with Midostaurin, results in a higher transduction efficiency compared to Lentiboost® alone. Lentiboost® may be used in combination with Midostaurin as a transduction enhancer at any suitable concentration. In certain embodiments, the combination of Lentiboost® and Midostaurin may comprise Lentiboost® at a concentration ranging from about 0.1 mg/mL to about 10 mg/mL and Midostaurin at a concentration ranging from about 50 to about 20,000 nM. In certain embodiments, Lentiboost may be added to a target cell at a final concentration ranging from about 0.1 μg/mL to about 10 mg/mL in combination with Midostaurin at a final concentration ranging from about 50 to about 20,000 nM. Preferably, Lentiboost® may be added to a target cell at a final concentration ranging from about 0.1 mg/mL to about 3 mg/mL in combination with Midostaurin at a final concentration ranging from about 50 to about 5,000 nM. More preferably, Lentiboost® may be added to a target cell at a final concentration ranging from about 0.5 mg/mL to about 2 mg/mL in combination with Midostaurin at a final concentration ranging from about 50 to about 500 nM. Most preferably, Lentiboost® may be added to a target cell at a final concentration of about 1 mg/mL in combination with Midostaurin at a final concentration of about 400 nM. Preferably, the combination of Lentiboost® and Midostaurin is contacted in the pre-stimulation or co-stimulation step with a hematopoietic cell, more preferably an HSC at any of the concentrations and/or densities disclosed above. In a certain embodiment, HSC at a concentration of 0.5 to 1 E6 cells/mL or at a density of 2E6/cm² may be pre-stimulated and/or co-stimulated with a combination of 1 mg/mL Lentiboost® and 100 nM of Midostaurin. In a certain embodiment, HSC at a concentration of 0.5 to 1 E6 cells/mL or at a density of 2E6/cm² may be pre-stimulated and/or co-stimulated with a combination of 1 mg/mL Lentiboost® and 200 nM of Midostaurin. In a certain embodiment, HSC at a concentration of 0.5 to 1 E6 cells/mL or at a density of 2E6/cm² may be pre-stimulated and/or co-stimulated with a combination of 1 mg/mL Lentiboost® and 400 nM of Midostaurin.

In certain embodiments, the invention relates to the method according to the invention, wherein the transduction enhancer is a combination of Poloxamer F108 and Amphotericin B.

It has been shown by the inventors that Poloxamer F108, when used in combination with Amphotericin B, results in a higher transduction efficiency compared to any of the two compounds alone. Poloxamer F108 may be used in combination with Amphotericin B as a transduction enhancer at any suitable concentration. In certain embodiments, the combination of Poloxamer F108 and Amphotericin B may comprise Poloxamer F108 at a concentration ranging from about 0.1 mg/mL to about 10 mg/mL and Amphotericin B at a concentration ranging from about 0.1 μg/mL to about 10 μg/mL. In certain embodiments, Poloxamer F108 may be added to a target cell at a final concentration ranging from about 0.1 mg/mL to about 10 mg/mL in combination with Amphotericin B at a final concentration ranging from about 0.1 μg/mL to about 10 μg/mL. Preferably, Poloxamer F108 may be added to a target cell at a final concentration ranging from about 0.1 mg/mL to about 3 mg/mL in combination with Amphotericin B at a final concentration ranging from about 0.1 μg/mL to about 3 μg/mL More preferably, Poloxamer F108 may be added to a target cell at a final concentration ranging from about 0.5 mg/mL to about 2 mg/mL in combination with Amphotericin B at a final concentration ranging from about 0.5 μg/mL to about 2 μg/mL. Most preferably, Poloxamer F108 may be added to a target cell at a final concentration of about 1 mg/mL in combination with Amphotericin B at a final concentration of about 0.75 μg/mL. Preferably, the combination of Poloxamer F108 and Amphotericin B is contacted in the pre-stimulation or co-stimulation step with a hematopoietic cell, more preferably an HSC at any of the concentrations and/or densities disclosed above. In a certain embodiment, HSC at a concentration of 0.5 to 1 E6 cells/mL or at a density of 2E6/cm² may be pre-stimulated and/or co-stimulated with a combination of 1 mg/mL Poloxamer F108 and 0.5 Kg/mL of Amphotericin B. In a certain embodiment, HSC at a concentration of 0.5 to 1 E6 cells/mL or at a density of 2E6/cm² may be pre-stimulated and/or co-stimulated with a combination of 1 mg/mL Poloxamer F108 and 0.75 μg/mL of Amphotericin B. In a certain embodiment, HSCs at a concentration of 0.5 to 1 E6 cells/mL or at a density of 2E6/cm² may be pre-stimulated and/or co-stimulated with a combination of 1 mg/mL Poloxamer F108 and 1 μg/mL of Amphotericin B.

In certain embodiments, the invention relates to the method according to the invention, wherein the transduction enhancer is a combination of Poloxamer F108 and Silibinin.

It has been shown by the inventors that Poloxamer F108, when used in combination with Silibinin, results in a higher transduction efficiency compared to any of the two compounds alone. Poloxamer F108 may be used in combination with Silibinin as a transduction enhancer at any suitable concentration. In certain embodiments, the combination of Poloxamer F108 and Silibinin may comprise Poloxamer F108 at a concentration ranging from about 0.1 mg/mL to about 10 mg/mL and Silibinin at a concentration ranging from about 0.1 to about 25 μM. In certain embodiments, Poloxamer F108 may be added to a target cell at a final concentration ranging from about 0.1 μg/mL to about 10 mg/mL in combination with Silibinin at a final concentration ranging from about 0.1 to about 25 μM. Preferably, Poloxamer F108 may be added to a target cell at a final concentration ranging from about 0.1 mg/mL to about 3 mg/mL in combination with Silibinin at a final concentration ranging from about 0.1 to about 10 μM. More preferably, Poloxamer F108 may be added to a target cell at a final concentration ranging from about 0.5 mg/mL to about 2 mg/mL in combination with Silibinin at a final concentration ranging from about 1 to about 10 μM. Most preferably, Poloxamer F108 may be added to a target cell at a final concentration of about 1 mg/mL in combination with Silibinin at a final concentration of about 5 μM. Preferably, the combination of Poloxamer F108 and Amphotericin B is contacted in the pre-stimulation or co-stimulation step with a hematopoietic cell, more preferably an HSC at any of the concentrations and/or densities disclosed above. In a certain embodiment, HSC at a concentration of 0.5 to 1 E6 cells/mL or at a density of 2E6/cm² may be pre-stimulated and/or co-stimulated with a combination of 1 mg/mL Poloxamer F108 and 5 μM Silibinin.

In certain embodiments, the invention relates to the method according to the invention, wherein the transduction enhancer is a combination of Poloxamer F108 and Midostaurin.

It has been shown by the inventors that Poloxamer F108, when used in combination with Midostaurin, results in a higher transduction efficiency compared to any of the two compounds alone. Poloxamer F108 may be used in combination with Midostaurin as a transduction enhancer at any suitable concentration. In certain embodiments, the combination of Poloxamer F108 and Midostaurin may comprise Poloxamer F108 at a concentration ranging from about 0.1 mg/mL to about 10 mg/mL and Midostaurin at a concentration ranging from about 50 to about 20,000 nM. In certain embodiments, Poloxamer F108 may be added to a target cell at a final concentration ranging from about 0.1 μg/mL to about 10 mg/mL in combination with Midostaurin at a final concentration ranging from about 50 to about 20,000 nM. Preferably, Poloxamer F108 may be added to a target cell at a final concentration ranging from about 0.1 mg/mL to about 3 mg/mL in combination with Midostaurin at a final concentration ranging from about 50 to about 5,000 nM. More preferably, Poloxamer F108 may be added to a target cell at a final concentration ranging from about 0.5 mg/mL to about 2 mg/mL in combination with Midostaurin at a final concentration ranging from about 50 to about 500 nM. Most preferably, Poloxamer F108 may be added to a target cell at a final concentration of about 1 mg/mL in combination with Midostaurin at a final concentration of about 400 nM. Preferably, the combination of Poloxamer F108 and Midostaurin is contacted in the pre-stimulation or co-stimulation step with a hematopoietic cell, more preferably an HSC at any of the concentrations and/or densities disclosed above. In a certain embodiment, HSC at a concentration of 0.5 to 1 E6 cells/mL or at a density of 2E6/cm² may be pre-stimulated and/or co-stimulated with a combination of 1 mg/mL Poloxamer F108 and 100 μM Midostaurin. In a certain embodiment, HSC at a concentration of 0.5 to 1 E6 cells/mL or at a density of 2E6/cm² may be pre-stimulated and/or co-stimulated with a combination of 1 mg/mL Poloxamer F108 and 200 μM Midostaurin. In a certain embodiment, HSC at a concentration of 0.5 to 1 E6 cells/mL or at a density of 2E6/cm² may be pre-stimulated and/or co-stimulated with a combination of 1 mg/mL Poloxamer F108 and 400 μM Midostaurin.

In certain embodiments, the invention relates to the method according to the invention, wherein the transduction enhancer is a combination of Silibinin and PEG-PCL-PEG.

It has been shown by the inventors that Silibinin, when used in combination with PEG-PCL-PEG, results in a higher transduction efficiency compared to any of the two compounds alone.

Silibinin may be used in combination with PEG-PCL-PEG as a transduction enhancer at any suitable concentration. In certain embodiments, the combination of Silibinin and PEG-PCL-PEG may comprise Silibinin at a concentration ranging from about 0.1 to about 25 μM and PEG-PCL-PEG at a concentration ranging from about 0.1 μg/ml to about 5,000 μg/ml. In certain embodiments, Silibinin may be added to a target cell at a final concentration ranging from about 0.1 to about 25 μM in combination with PEG-PCL-PEG at a final concentration ranging from about 0.1 μg/ml to about 5,000 μg/ml. Preferably, Silibinin may be added to a target cell at a final concentration ranging from about 0.1 μM to about 10 μM in combination with PEG-PCL-PEG at a final concentration ranging from about 0.1 to about 5,000 μg/ml. More preferably, Silibinin may be added to a target cell at a final concentration ranging from about 1 to about 10 μM in combination with PEG-PCL-PEG at a final concentration ranging from about 5 μg/ml to about 1,000 μg/ml. Most preferably, Silibinin may be added to a target cell at a final concentration of about 5 μM in combination with PEG-PCL-PEG at a final concentration of about 10 μg/ml. Preferably, the combination of Silibinin and PEG-PCL-PEG is contacted in the pre-stimulation or co-stimulation step with a hematopoietic cell, more preferably an HSC at any of the concentrations and/or densities disclosed above. In a certain embodiment, HSC at a concentration of 0.5 to 1 E6 cells/mL or at a density of 2E6/cm² may be pre-stimulated and/or co-stimulated with a combination of 5 μM Silibinin and 10 μg/ml of PEG-PCL-PEG.

In certain embodiments, the invention relates to the method according to the invention, wherein the transduction enhancer is a combination of Amphotericin B and Everolimus.

It has been shown by the inventors that Amphotericin B, when used in combination with Everolimus, results in a higher transduction efficiency compared to any of the two compounds alone. Amphotericin B may be used in combination with Everolimus as a transduction enhancer at any suitable concentration. In certain embodiments, the combination of Amphotericin B and Everolimus may comprise Amphotericin B at a concentration ranging from about 0.1 μg/mL to about 10 μg/mL and Everolimus at a concentration ranging from about 0.1 to about 10 μM. In certain embodiments, Amphotericin B may be added to a target cell at a final concentration ranging from about 0.1 μg/mL to about 10 μg/mL in combination with Everolimus at a final concentration ranging from about 0.1 to about 10 μM. Preferably, Amphotericin B may be added to a target cell at a final concentration ranging from about 0.1 μg/mL to about 3 μg/mL in combination with Everolimus at a final concentration ranging from about 0.2 to about 7.5 μM. More preferably, Amphotericin B may be added to a target cell at a final concentration ranging from about 0.5 μg/mL to about 2 μg/mL in combination with Everolimus at a final concentration ranging from about 0.5 to about 5 μM. Most preferably, Amphotericin B may be added to a target cell at a final concentration of about 1 μg/mL in combination with Everolimus at a final concentration of about 1 μM. Preferably, the combination of Amphotericin B and Everolimus is contacted in the pre-stimulation or co-stimulation step with a hematopoietic cell, more preferably an HSC at any of the concentrations and/or densities disclosed above. In a certain embodiment, HSC at a concentration of 0.5 to 1 E6 cells/mL or at a density of 2E6/cm² may be pre-stimulated and/or co-stimulated with a combination of 0.5 μg/mL Amphotericin B and 1 μM of Everolimus. In a certain embodiment, HSC at a concentration of 0.5 to 1 E6 cells/mL or at a density of 2E6/cm² may be pre-stimulated and/or co-stimulated with a combination of 0.75 μg/mL Amphotericin B and 1 μM of Everolimus. In a certain embodiment, HSC at a concentration of 0.5 to 1 E6 cells/mL or at a density of 2E6/cm² may be pre-stimulated and/or co-stimulated with a combination of 1 μg/mL Amphotericin B and 1 μM of Everolimus.

In certain embodiments the invention relates to a combination of a protamine salt with one or more of the transduction enhancers disclosed herein for use as a transduction enhancer.

“Protamine” as used herein refers to the generic name of a group of strongly basic proteins present in sperm cells in salt-like combination with nucleic acids. Protamines may be obtained from e.g. salmon (salmine), rainbow trout (iridine), herring (clupeine), sturgeon (sturine), or Spanish mackerel or tuna (thynnine) and a wide variety of salts of protamines are commercially available. It is to be understood that the peptide composition of a specific protamine may vary depending of which family, genera or species of fish it is obtained from. Protamine usually contains four major components, i.e. single-chain peptides containing about 30 to 32 residues of which about 21 to 22 are Arginine residues. The N-terminal is proline for each of the four main components, and since no other amino groups are present in the sequence, chemical modification of protamine by a particular salt is expected to be homogenous in this context.

Within the present invention, the protamine salt to be used in the method of the invention may include, but is not limited to, chloride, sulfate, acetate, bromide, caproate, trifluoroacetate, HCO₃, propionate, lactate, formiate, nitrate, citrate, monohydrogenphosphate, dihydrogenphosphate, tartrate, or perchlorate salts of protamine or mixtures of any two protamine salts.

In one embodiment, the protamine salts used in the method of the present invention are from salmon. In another embodiment, the protamine salts used in the method of the present invention are from herring. In yet another embodiment, the protamine salts used in the method of the present invention are from rainbow trout. In another embodiment, the protamine salts used in the method of the present invention are from tuna.

Protamine may be added to the pre- and/or co-incubation medium in any salt form, provided that the anionic component of the salt does not inhibit transduction efficiency of the target cell when in solution. Preferable protamine salts that may be used in the method of the invention are protamine chloride and protamine sulfate. In a certain embodiment, protamine is added to the pre- and/or co-incubation medium as GMP-grade protamine chloride.

It has been shown by the inventors that protamine salts enhance the transduction efficiency of a target cell when contacted with the target cell at a concentration of 4 μg/mL. Thus, in a certain embodiment, protamine salts may be used as a transduction enhancer at a concentration ranging from about 0.05 μg/mL to about 25 μg/mL, preferably at a concentration ranging from about 0.1 μg/mL to about 10 μg/mL, more preferably at a concentration ranging from about 1 μg/mL to about 10 μg/mL, most preferably at a concentration of about 4 μg/mL. Preferably, protamine salts are contacted in the pre-stimulation and/or co-stimulation step with a hematopoietic cell, more preferably an HSC at any of the concentrations and/or densities disclosed above. In a certain embodiment, HSC at a concentration of 0.5 to 1 E6 cells/mL or in a density of 2E6/cm² may be pre-stimulated and/or co-stimulated with protamine salts at a concentration of 4 μg/mL.

In certain embodiments, the invention relates to the method according to the invention, wherein the transduction enhancer is a combination of a protamine salt and Amphotericin B.

It has been shown by the inventors that protamine salts, when used in combination with Amphotericin B, result in a higher transduction efficiency compared to any of the two compounds alone. Protamine salts may be used in combination with Amphotericin B as a transduction enhancer at any suitable concentration. In certain embodiments, the combination of a protamine salt and Amphotericin B may comprise a protamine salt at a concentration ranging from about 0.05 μg/mL to about 25 μg/mL and Amphotericin B at a concentration ranging from about 0.1 μg/mL to about 10 μg/mL In certain embodiments, the protamine salt may be added to a target cell at a final concentration ranging from about 0.05 μg/mL to about 25 μg/mL in combination with Amphotericin B at a final concentration ranging from about 0.1 μg/mL to about 10 μg/mL. Preferably, a protamine salt may be added to a target cell at a final concentration ranging from about 0.1 μg/mL to about 10 μg/mL in combination with Amphotericin B at a final concentration ranging from about 0.1 μg/mL to about 3 μg/mL. More preferably, a protamine salt may be added to a target cell at a final concentration ranging from about 1 μg/mL to about 10 μg/mL in combination with Amphotericin B at a final concentration ranging from about 0.5 μg/mL to about 2 μg/mL. Most preferably, a protamine salt may be added to a target cell at a final concentration of about 4 μg/mL in combination with Amphotericin B at a final concentration of about 1 μg/mL. Preferably, the combination of a protamine salt and Amphotericin B is contacted in the pre-stimulation and/or co-stimulation step with a hematopoietic cell, more preferably an HSC at any of the concentrations and/or densities disclosed above. In a certain embodiment, HSC at a concentration of 0.5 to 1 E6 cells/mL or in a density of 2E6/cm² may be pre-stimulated and/or co-stimulated with a combination of 4 μg/mL of a protamine salt and 0.5 μg/mL of Amphotericin B. In a certain embodiment, HSC at a concentration of 0.5 to 1 E6 cells/mL or at a density of 2E6/cm² may be pre-stimulated and/or co-stimulated with a combination of 4 μg/mL of a protamine salt and 0.75 μg/mL of Amphotericin B. In a certain embodiment, HSC at a concentration of 0.5 to 1 E6 cells/mL or at a density of 2E6/cm² may be pre-stimulated and/or co-stimulated with a combination of 4 μg/mL of a protamine salt and 1 μg/mL of Amphotericin B.

In certain embodiments, the invention relates to the method according to the invention, wherein the transduction enhancer is a combination of a protamine salt and PEG-PCL-PEG.

It has been shown by the inventors that protamine salts, when used in combination with PEG-PCL-PEG, result in a higher transduction efficiency compared to any of the two compounds alone. Protamine salts may be used in combination with PEG-PCL-PEG as a transduction enhancer at any suitable concentration. In certain embodiments, the combination of a protamine salt and PEG-PCL-PEG may comprise a protamine salt at a concentration ranging from about 0.05 μg/mL to about 25 μg/mL and PEG-PCL-PEG at a concentration ranging from about 0.1 ng/ml to about 5,000 μg/ml. In certain embodiments, the protamine salt may be added to a target cell at a final concentration ranging from about 0.05 μg/mL to about 25 μg/mL in combination with PEG-PCL-PEG at a final concentration ranging from about 0.1 μg/ml to about 5,000 μg/ml. Preferably, a protamine salt may be added to a target cell at a final concentration ranging from about 0.1 μg/mL to about 10 μg/mL in combination with PEG-PCL-PEG at a final concentration ranging from about 1 μg/ml to about 2,500 μg/ml. More preferably, a protamine salt may be added to a target cell at a final concentration ranging from about 1 μg/mL to about 10 μg/mL in combination with PEG-PCL-PEG at a final concentration ranging from about 5 μg/ml to about 1,000 μg/ml. Most preferably, a protamine salt may be added to a target cell at a final concentration of about 4 μg/mL in combination with PEG-PCL-PEG at a final concentration of about 10 μg/ml. Preferably, the combination of a protamine salt and PEG-PCL-PEG is contacted in the pre-stimulation and/or co-stimulation step with a hematopoietic cell, more preferably an HSC at any of the concentrations and/or densities disclosed above. In a certain embodiment, HSC at a concentration of 0.5 to 1 E6 cells/mL or at a density of 2E6/cm² may be pre-stimulated and/or co-stimulated with a combination of 4 μg/mL of a protamine salt and 10 μg/ml of PEG-PCL-PEG.

That is, in certain embodiments, the invention relates to the method according to the invention, wherein the transduction enhancer is a combination of a protamine salt and Silibinin.

It has been shown by the inventors that protamine salts, when used in combination with Silibinin, result in a higher transduction efficiency compared to any of the two compounds alone. Protamine salts may be used in combination with Silibinin as a transduction enhancer at any suitable concentration. In certain embodiments, the combination of a protamine salt and Silibinin may comprise a protamine salt at a concentration ranging from about 0.05 μg/mL to about 25 μg/mL and Silibinin at a concentration ranging from about 0.1 to about 25 μM. In certain embodiments, the protamine salt may be added to a target cell at a final concentration ranging from about 0.05 μg/mL to about 25 μg/mL in combination with Silibinin at a final concentration ranging from about 0.1 to about 25 μM. Preferably, a protamine salt may be added to a target cell at a final concentration ranging from about 0.1 μg/mL to about 10 μg/mL in combination with Silibinin at a final concentration ranging from about 0.1 to about 10 μM. More preferably, a protamine salt may be added to a target cell at a final concentration ranging from about 1 μg/mL to about 10 μg/mL in combination with Silibinin at a final concentration ranging from about 1 to about 10 μM. Most preferably, a protamine salt may be added to a target cell at a final concentration of about 4 μg/mL in combination with Silibinin at a final concentration of about 5 μM. Preferably, the combination of a protamine salt and Silibinin is contacted in the pre-stimulation and/or co-stimulation step with a hematopoietic cell, more preferably an HSC at any of the concentrations and/or densities disclosed above. In a certain embodiment, HSC at a concentration of 0.5 to 1 E6 cells/mL or at a density of 2E6/cm² may be pre-stimulated and/or co-stimulated with a combination of 4 μg/mL of a protamine salt and 5 μM of Silibinin.

In certain embodiments, the invention relates to the method according to the invention, wherein the transduction enhancer is a combination of a protamine salt and Resveratrol.

It has been shown by the inventors that protamine salts, when used in combination with Resveratrol, result in a higher transduction efficiency compared to any of the two compounds alone. Protamine salts may be used in combination with Resveratrol as a transduction enhancer at any suitable concentration. In certain embodiments, the combination of a protamine salt and Resveratrol may comprise a protamine salt at a concentration ranging from about 0.05 μg/mL to about 25 μg/mL and Resveratrol at a concentration ranging from about 0.1 to about 10 μM. In certain embodiments, the protamine salt may be added to a target cell at a final concentration ranging from about 0.05 μg/mL to about 25 μg/mL in combination with Resveratrol at a final concentration ranging from about 0.1 to about 25 μM. Preferably, a protamine salt may be added to a target cell at a final concentration ranging from about 0.1 μg/mL to about 10 μg/mL in combination with Resveratrol at a final concentration ranging from about 1 to about 7.5 μM. More preferably, a protamine salt may be added to a target cell at a final concentration ranging from about 1 μg/mL to about 10 μg/mL in combination with Resveratrol at a final concentration ranging from about 2.5 to about 7.5 μM. Most preferably, a protamine salt may be added to a target cell at a final concentration of about 4 μg/mL in combination with Resveratrol at a final concentration of about 5 μM. Preferably, the combination of a protamine salt and Resveratrol is contacted in the pre-stimulation and/or co-stimulation step with a hematopoietic cell, more preferably an HSC at any of the concentrations and/or densities disclosed above. In a certain embodiment, HSC at a concentration of 0.5 to 1 E6 cells/mL or at a density of 2E6/cm² may be pre-stimulated and/or co-stimulated with a combination of 4 μg/mL of a protamine salt and 5 μM of Resveratrol.

In certain embodiments, the invention relates to the method according to the invention, wherein the transduction enhancer is a combination of a protamine salt and Midostaurin.

It has been shown by the inventors that protamine salts, when used in combination with Midostaurin, result in a higher transduction efficiency compared to any of the two compounds alone. Protamine salts may be used in combination with Midostaurin as a transduction enhancer at any suitable concentration. In certain embodiments, the combination of a protamine salt and Midostaurin may comprise a protamine salt at a concentration ranging from about 0.05 μg/mL to about 25 μg/mL and Midostaurin at a concentration ranging from about 50 to about 20,000 nM. In certain embodiments, the protamine salt may be added to a target cell at a final concentration ranging from about 0.05 μg/mL to about 25 μg/mL in combination with Midostaurin at a final concentration ranging from about 50 to about 20,000 nM. Preferably, a protamine salt may be added to a target cell at a final concentration ranging from about 0.1 μg/mL to about 10 μg/mL in combination with Midostaurin at a final concentration ranging from about 50 to about 5,000 nM. More preferably, a protamine salt may be added to a target cell at a final concentration ranging from about 1 μg/mL to about 10 μg/mL in combination with Midostaurin at a final concentration ranging from about 50 to about 500 nM. Most preferably, a protamine salt may be added to a target cell at a final concentration of about 4 μg/mL in combination with Midostaurin at a final concentration of about 400 nM. Preferably, the combination of a protamine salt and Midostaurin is contacted in the pre-stimulation and/or co-stimulation step with a hematopoietic cell, more preferably an HSC at any of the concentrations and/or densities disclosed above. In a certain embodiment, HSC at a concentration of 0.5 to 1 E6 cells/mL or at a density of 2E6/cm² may be pre-stimulated and/or co-stimulated with a combination of 4 μg/mL of a protamine salt and 100 nM of Midostaurin. In a certain embodiment, HSC at a concentration of 0.5 to 1 E6 cells/mL or at a density of 2E6/cm² may be pre-stimulated and/or co-stimulated with a combination of 4 μg/mL of a protamine salt and 200 nM of Midostaurin. In a certain embodiment, HSC at a concentration of 0.5 to 1E6 cells/mL or at a density of 2E6/cm² may be pre-stimulated and/or co-stimulated with a combination of 4 μg/mL of a protamine salt and 400 nM of Midostaurin.

In certain embodiments, the invention relates to the method according to the invention, wherein the transduction enhancer is a combination of a protamine salt and Nystatin.

It has been shown by the inventors that protamine salts, when used in combination with Nystatin, result in a higher transduction efficiency compared to any of the two compounds alone. Protamine salts may be used in combination with Nystatin as a transduction enhancer at any suitable concentration, In certain embodiments, the combination of a protamine salt and Nystatin may comprise a protamine salt at a concentration ranging from about 0.05 μg/mL to about 25 μg/mL and Nystatin at a concentration ranging from about 1 to about 1,000 μM. In certain embodiments, the protamine salt may be added to a target cell at a final concentration ranging from about 0.05 μg/mL to about 25 μg/mL in combination with Nystatin at a final concentration ranging from about 1 to about 1,000 μM. Preferably, a protamine salt may be added to a target cell at a final concentration ranging from about 0.1 μg/mL to about 10 μg/mL in combination with Nystatin at a final concentration ranging from about 5 to about 500 μM. More preferably, a protamine salt may be added to a target cell at a final concentration ranging from about 1 μg/mL to about 10 μg/mL in combination with Nystatin at a final concentration ranging from about 50 to about 150 μM. Most preferably, a protamine salt may be added to a target cell at a final concentration of about 4 μg/mL in combination with Nystatin at a final concentration of about 100 μM. Preferably, the combination of a protamine salt and Nystatin is contacted in the pre-stimulation and/or co-stimulation step with a hematopoietic cell, more preferably an HSC at any of the concentrations and/or densities disclosed above. In a certain embodiment, HSC at a concentration of 0.5 to 1 E6 cells/mL or at a density of 2E6/cm² may be pre-stimulated and/or co-stimulated with a combination of 4 μg/mL of a protamine salt and 100 μM of Nystatin.

In certain embodiments, the invention relates to the method according to the invention, wherein the transduction enhancer is a combination of a protamine salt and Natamycin.

It has been shown by the inventors that protamine salts, when used in combination with Natamycin, result in a higher transduction efficiency compared to any of the two compounds alone. Protamine salts may be used in combination with Natamycin as a transduction enhancer at any suitable concentration. In certain embodiments, the combination of a protamine salt and Natamycin may comprise protamine at a concentration ranging from about 0.05 μg/mL to about 25 μg/mL and Natamycin at a concentration ranging from about 0.05 to about 500 μM. In certain embodiments, the protamine salt may be added to a target cell at a final concentration ranging from about 0.05 μg/mL to about 25 μg/mL in combination with Natamycin at a final concentration ranging from about 0.05 to about 500 μM. Preferably, a protamine salt may be added to a target cell at a final concentration ranging from about 0.1 μg/mL to about 10 μg/mL in combination with Natamycin at a final concentration ranging from about 0.05 to about 10 μM. More preferably, a protamine salt may be added to a target cell at a final concentration ranging from about 1 μg/mL to about 10 μg/mL in combination with Natamycin at a final concentration ranging from about 1 to about 5 μM. Most preferably, a protamine salt may be added to a target cell at a final concentration of about 4 μg/mL in combination with Natamycin at a final concentration of about 3 μM. Preferably, the combination of a protamine salt and Natamycin is contacted in the pre-stimulation and/or co-stimulation step with a hematopoietic cell, more preferably an HSC at any of the concentrations and/or densities disclosed above. In a certain embodiment, HSC at a concentration of 0.5 to 1 E6 cells/mL or at a density of 2E6/cm² may be pre-stimulated and/or co-stimulated with a combination of 4 μg/mL of a protamine salt and 3 μM of Natamycin.

In certain embodiments, the invention relates to the method according to the invention, wherein the transduction enhancer is a combination of a protamine salt, Amphotericin B and Everolimus.

It has been shown by the inventors that protamine salts, when used in combination with Amphotericin B and Everolimus, result in a higher transduction efficiency compared to any of the compounds alone. Protamine salts may be used in combination with Amphotericin B and Everolimus as a transduction enhancer at any suitable concentration. In certain embodiments, the combination of a protamine salt, Amphotericin B and Everolimus may comprise protamine at a concentration ranging from about 0.05 μg/mL to about 25 μg/mL, Amphotericin B at a concentration ranging from about 0.1 μg/mL to about 10 μg/mL and Everolimus at a concentration ranging from about 0.1 to about 10 μM. In certain embodiments, the protamine salt may be added to a target cell at a final concentration ranging from about 0.05 μg/mL to about 25 μg/mL in combination with Amphotericin B at a final concentration ranging from about 0.1 μg/mL to about 10 μg/mL and Everolimus at a final concentration ranging from about 0.1 to about 10 μM. Preferably, a protamine salt may be added to a target cell at a final concentration ranging from about 0.1 μg/mL to about 10 μg/mL in combination with Amphotericin B at a final concentration ranging from about 0.1 μg/mL to about 3 μg/mL and Everolimus at a final concentration ranging from about 0.2 to about 7.5 μM. More preferably, a protamine salt may be added to a target cell at a final concentration ranging from about 1 μg/mL to about 10 μg/mL in combination with Amphotericin B at a final concentration ranging from about 0.5 μg/mL to about 2 μg/mL and Everolimus at a final concentration ranging from about 0.5 to about 5 μM. Most preferably, a protamine salt may be added to a target cell at a final concentration of about 4 μg/mL in combination with Amphotericin B at a final concentration of about 1 μg/mL and Everolimus at a final concentration of about 1 μM. Preferably, the combination of a protamine salt, Amphotericin B and Everolimus is contacted in the pre-stimulation and/or co-stimulation step with a hematopoietic cell, more preferably an HSC at any of the concentrations disclosed above. In a certain embodiment, HSC at a concentration of 0.5 to 1 E6 cells/mL or at a density of 2E6/cm² may be pre-stimulated and/or co-stimulated with a combination 4 μg/mL of a protamine salt, 0.5 μg/mL Amphotericin B and 1 μM of Everolimus. In a certain embodiment, HSC at a concentration of 0.5 to 1 E6 cells/mL or at a density of 2E6/cm² may be pre-stimulated and/or co-stimulated with a combination 4 μg/mL of a protamine salt, 0.75 μg/mL Amphotericin B and 1 μM of Everolimus. In a certain embodiment, HSC at a concentration of 0.5 to 1 E6 cells/mL or at a density of 2E6/cm² may be pre-stimulated and/or co-stimulated with a combination 4 μg/mL of a protamine salt, 1 μg/mL Amphotericin B and 1 μM of Everolimus.

Several compounds and combination of compounds could be identified, which enhance the transduction of human cells by gene therapy vectors. In particular, novel compounds could be identified, which were shown to mediate an increase in retroviral transduction efficacy of target cells, particularly of human CD34-positive HSC, when brought into contact with a retroviral vector, particularly a lentiviral self-inactivating (SIN) vector, comprising a transgene of interest, particularly the p47phox encoding cDNA, under control of a internal promoter, such as, for example, the myelospecific miR223 promoter, simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters, but particularly the myelospecific miR223 promoter.

In a specific embodiment of the invention, a lentiviral Self-Inactivating (SIN) vector may be used within the method of the present invention, wherein on plasmid level, the viral promoter/enhancer was deleted within the 3′ long terminal repeat (LTR). Expression of the transgene of interest may be driven by an internal promoter, such as, for example, simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters or the myelospecific miR223 promoter, but particularly the myelospecific miR223 promoter.

In a specific embodiment of the invention, human CD34-positive HSCs are transduced by a lentiviral self-inactivating gene therapy vector, comprising cDNA under control of the miR223 promoter encoding p47phox.

In a specific embodiment of the invention, the pre-incubation medium may be further supplemented with protamine sulfate or protamine chloride, preferably at the concentration indicated herein. In a specific embodiment of the invention, the co-incubation medium may be further supplemented with protamine sulfate or protamine chloride, preferably at the concentration indicated herein. In a specific embodiment of the invention, the pre- and/or co-incubation medium may be supplemented with 4 μg,/mL protamine sulfate or protamine chloride.

In another specific embodiment, the pre-incubation or co-incubation medium may be further supplemented with polybrene, preferably at a concentration ranging from about 0.1 to about 20 μg/mL, and/or poly-L-lysine, preferably at a concentration ranging from about 0.1 to about 20 μg/mL.

In various embodiments of the invention the transduction enhancing compound is one selected from the group consisting of Silibinin, particularly in a concentration of 5 μM, Resveratrol, particularly in a concentration of 5 μM, Everolimus, particularly in a concentration of 1 μM, Midostaurin, particularly in a concentration of 0,4 μM, Amphotericin B, particularly in a concentration of 1 μM, Nystatin, particularly in a concentration of 100 μM, Natamycin, particularly in a concentration of 3 μM, Prostaglandin E2, particularly in a concentration of 10 μM, Poloxamer Symperonic F108®, particularly in a concentration of 1,000 μg/ml, poly(ethylene glycol)-b-poly(D,L-lactic acid-co-glycolic acid)-b-poly(ethylene glycol) (PEG-PLGA-PEG) with 5 kDa poly(ethylene glycol) block on both ends, and a central 4.2 kDa poly(D,L-lactic acid-co-glycolic acid) block, termed PEG5k-b-PLGA4.2k-b-PEG5k, particularly in a concentration of 1,000 μg/ml, methoxypoly(ethylene glycol)-poly(e-caprolacton)-methoxypoly(ethylene glycol) (PEG-PCL-PEG) with 5 kDa poly(ethylene glycol) block on both ends, and a central 4.2 kDa poly(e-caprolacton) block, termed PEG5k-b-PCL4.2k-b-PEG5k, particularly in a concentration of 10 μg/ml, methoxypoly(ethylene glycol)-poly(e-caprolacton)-methoxypoly(ethylene glycol) (PEG-PCL-PEG) with a central 2.4 kDa poly(e-caprolacton) block, and a 5.3 kDa poly(ethylene glycol) block on both ends, which both were covalently linked to an amino group, termed NH2-PEG5.3k-b- PCL2.4k-b-PEG5.3k-NH2, particularly in a concentration of 10 μW 1, poly(ethylene glycol)/poly(lactide)/poly(ethylene glycol) (PEG-PLA-PEG) with 5 kDa poly(ethylene glycol) blocks on both ends, and a central 4.2 kDa poly(lactide) block, termed PEG5k-b-PLA4.2k-b-PEG5k, particularly in a concentration of 50 ng/ml and deoxyribonucleosides, particularly with a final concentration of 300 μM of each, or with combinations thereof.

Lentiboost® is widely acknowledged as the compound of choice when transducing human cells with retroviral vectors, in particular, lentiviral vectors. However, even though it is or has been the subject of various clinical trials, Lentiboost®, as of today, has not obtained regulatory approval for therapeutic uses. Further, Lentiboost® comprises synthetic polymers and thus bears the risk that non-degradable compounds accumulate in cells that have been treated with Lentiboost® with so far unforeseeable consequences for human beings. Accordingly, there is a need in the art for safer transduction enhancers.

The inventors have surprisingly shown that several compounds that have been approved for therapeutic use in human are well suited as transduction enhancers and, thus, may be preferred over Lentiboost® for use in therapeutic applications.

For example, it has been shown by the inventors that the approved therapeutic compounds Silibinin (Legalon), Midostaurin (Rydapt), Amphotericin B (AmBisome), Nystatin (Mycostatin), Natamycin (Natacyn), Ruxolitinib (Jakavi), Fludarabine (Fludara) are efficient transduction enhancers. Thus, in a particular embodiment, the invention relates to the method according to the invention, wherein the transduction enhancer is Silibinin, Midostaurin, Amphotericin B, Nystatin, Natamycin, Ruxolitinib, Fludarabine or any combination thereof. In a preferred embodiment, the invention relates to the method according to the invention, wherein the transduction enhancer is Silibinin, Midostaurin, Amphotericin B, Nystatin, Natamycin, Ruxolitinib, Fludarabine or any combination thereof, in particular wherein the combination is a combination of Everolimus and Amphotericin B. In certain embodiments, Silibinin, Midostaurin, Amphotericin B, Nystatin, Natamycin, Ruxolitinib, Fludarabine or any combination thereof may be combined with a protamine salt, in particular protamine sulfate or protamine chloride at any of the concentrations disclosed herein.

In a particular embodiment, the invention relates to the method according to the invention, wherein the transduction enhancer is Silibinin, Midostaurin, Amphotericin B, Nystatin, Natamycin or any combination thereof. In another embodiment, the invention relates to the method according to the invention, wherein the transduction enhancer is Silibinin, Everolimus, Midostaurin, Amphotericin B, Nystatin, Natamycin or any combination thereof, in particular wherein the combination is a combination of Everolimus and Amphotericin B. In certain embodiments, Silibinin, Midostaurin, Amphotericin B, Nystatin, Natamycin or any combination thereof may be combined with a protamine salt, in particular protamine sulfate or protamine chloride at any of the concentrations disclosed herein.

Further, the inventors have identified that certain compounds can increase the transduction efficiency of Lentiboost®. In particular, the inventors have surprisingly found that the combination of Lentiboost® with Amphotericin B, Silibinin and/or Midostaurin results in increased transduction efficiencies compared to Lentiboost® alone. Thus, in a particular embodiment, the invention relates to the method according to the invention, wherein the transduction enhancer is Lentiboost® in combination with Amphotericin B, Silibinin and/or Midostaurin. In another embodiment, the invention relates to the method according to the invention, wherein the transduction enhancer is Lentiboost® in combination with Amphotericin B and/or Midostaurin. In another embodiment, the invention relates to the method according to the invention, wherein the transduction enhancer is a combination of Lentiboost® with Amphotericin B or a combination of Lentiboost® with Midostaurin or a combination with Lentiboost® and Silibinin. Lentiboost® may be combined with Amphotericin B, Silibinin and/or Midostaurin at any of the concentrations disclosed herein.

In addition, the inventors have found that specific combinations of transduction enhancers result in an increased transduction efficiency compared to Lentiboost® when used at its recommended concentration of 1 mg/mL.

The inventors found that transduction of HSC with a lentiviral vector at an MOI of 10 resulted at VCNs of 3.5 in X-Vivo 10 medium and 5 in BESP1366F medium when pre- and co-incubated with 1 mg/mL Lentiboost® (see FIGS. 2 and 3).

To the surprise of the inventors, the combination of a protamine salt and Amphotericin B resulted in a VCN of 5.4 in BESP1366F medium when under the same conditions as with Lentiboost® (see FIG. 4). Thus, in a particular embodiment, the invention relates to the method according to the invention, wherein the transduction enhancer is a protamine salt in combination with Amphotericin B.

Further, the inventors found that the combination of a protamine salt and PEG-PCL-PEG resulted in a VCN of 4 in X-Vivo 10 medium when treated under the same conditions as with Lentiboost® (see FIG. 6). Thus, in a particular embodiment, the invention relates to the method according to the invention, wherein the transduction enhancer is a protamine salt in combination with PEG-PCL-PEG.

Further, the inventors found that the combination of Amphotericin B and poloxamer F108 resulted in a VCN of 6.8 in X-Vivo 10 medium when treated under the same conditions as with Lentiboost® (see FIG. 8). Thus, in a particular embodiment, the invention relates to the method according to the invention, wherein the transduction enhancer is Amphotericin B in combination with poloxamer F108.

Further, the inventors found that the combination of Silibinin and PEG-PCL-PEG resulted in a VCN of 5 in X-Vivo 10 medium when treated under the same conditions as with Lentiboost® (see FIG. 9). Thus, in a particular embodiment, the invention relates to the method according to the invention, wherein the transduction enhancer is Silibinin in combination with PEG-PCL-PEG.

Further, the inventors found that the combination of Silibinin and poloxamer F108 resulted in a VCN of 7.2 in X-Vivo 10 medium when treated under the same conditions as with Lentiboost® (see FIG. 9). Thus, in a particular embodiment, the invention relates to the method according to the invention, wherein the transduction enhancer is Silibinin in combination with poloxamer F108.

Further, the inventors found that the combination of Midostaurin and poloxamer F108 resulted in a VCN of 9.7 in X-Vivo 10 medium when treated under the same conditions as with Lentiboost® (see FIG. 10). Thus, in a particular embodiment, the invention relates to the method according to the invention, wherein the transduction enhancer is Midostaurin in combination with poloxamer F108.

Thus, in a particular embodiment, the invention relates to the method according to the invention, wherein the transduction enhancer is a protamine salt in combination with amphotericin B, a protamine salt in combination with PEG-PCL-PEG, Amphotericin B in combination with poloxmer F108, Silibinin in combination with PEG-PCL-PEG, Silibinin in combination with poloxamer F108 or Midostaurin in combination with poloxamer F108, preferably at any of the concentrations disclosed herein.

In a particular embodiment, the invention relates to the method according to the invention, wherein the transduction enhancer is a protamine salt in combination with amphotericin B, a protamine salt in combination with PEG-PCL-PEG, Amphotericin B in combination with poloxmer F108, Silibinin in combination with PEG-PCL-PEG, Silibinin in combination with poloxamer F108, Midostaurin in combination with poloxamer F108, Lentiboost® in combination with Amphotericin B, Lentiboost® in combination with Silibinin or Lentiboost® in combination with Midostaurin, preferably at any of the concentrations disclosed herein.

In a particular embodiment the invention relates to the method according to the invention, wherein the transduction enhancing compound is selected from a group consisting of:
Silibinin, in particular at a concentration between 0.05 μM and 500 μM;
Midostaurin, in particular at a concentration between 2 nM and 500,000 nM;
Nystatin, in particular at a concentration between 0.1 and 1000 μM;
Natamycin, in particular at a concentration between 0.05 and 500 μM;
a PEG-PCL-PEG polymer, in particular at a concentration between 1 μg/ml and 5,000 μg/ml;
a PEG-PLGA-PEG polymer, in particular at a concentration between 1 μg/ml and 5,000 μg/ml;
a PEG-PLA-PEG polymer, in particular at a concentration between 1 μg/ml and 5,000 μg/ml; and/or
any combination thereof.

Further, the inventors have surprisingly found that Amphotericin B can be used as a transduction enhancer, which has not been suggested before. Thus, in a particular embodiment, the invention relates to the method according to the invention, wherein the transduction enhancing compound is Amphotericin B.

Amphotericin B may be contacted with the target cell to induce transduction efficiency with a retroviral vector during the pre- and/or co-incubation step at any of the concentrations disclosed herein. Thus, in a particular embodiment, the invention relates to the method according to the invention, wherein the target cell is contacted with Amphotericin B during the pre-incubation and/or co-incubation step at a concentration of about 0.05 to about 500 μM, in particular at a concentration of about 0.1 to about 10 μM, or any of the concentrations disclosed herein.

Further, Amphotericin B may be combined with any transduction enhancing compound known in the art or any of the transduction enhancing compounds described herein, preferably at any of the concentrations disclosed herein. Thus, in a particular embodiment, the invention relates to the method according to the invention, wherein Amphotericin B is used in combination with one or more additional transduction enhancing compounds.

In certain embodiments, Amphotericin B may be used in combination with a protamine salt to improve the transduction efficiency of a target cell with a retroviral vector. Thus, in certain embodiments, the invention relates to the method according to the invention, wherein the additional transduction enhancing compound is a protamine salt.

The protamine salt may be any protamine salt known in the art, provided that the anionic component of the salt does not interfere with the transduction efficiency of the target cell when in solution. Thus, in a particular embodiment, the invention relates to the methods according to the invention, wherein the protamine salt is protamine chloride or protamine sulfate.

The protamine salt may be contacted with the target cell at any concentration disclosed herein. That is, in a certain embodiment, the invention related to the method according to the invention, wherein the target cell is contacted with the protamine salt during the pre-incubation and/or co-incubation step at a concentration of about 0.05 μg/mL to about 25 μg/mL, in particular at a concentration of about 0.1 μg/mL to about 10 μg/mL.

That is, in a preferred embodiment, the invention relates to the method according to the invention, wherein the target cell is contacted with a combination of Amphotericin B and a protamine salt during the pre-incubation and/or co-incubation step, wherein Amphotericin is contacted with the target cell at a concentration of about 0.05 to about 500 μM, in particular at a concentration of about 0.1 to about 10 μM, and wherein the protamine salt is contacted with the target cell at a concentration of about 0.05 μg/mL to about 25 μg/mL, in particular at a concentration of about 0.1 μg/mL to about 10 μg/mL.

When two or more transduction enhancing compounds are added to a target cell in combination, it is preferred that all compounds are present in the pre- and/or co-incubation medium simultaneously. However, it has to be noted that the two or more transduction enhancing compounds may be added to the pre- and/or co-incubation medium sequentially, as long as the compounds that are used in combination are simultaneously present in the pre-and/or co-incubation step at least at one time point during the pre- and/or co-incubation step.

It has further been shown that the transduction efficiency of Amphotericin B may be further increased when used in combination with one or more additional transduction enhancing compound. Thus, in a particular embodiment, the invention relates to the method according to the invention, wherein the one or more additional transduction enhancing compound is selected from the group consisting of:
Lentiboost®, in particular at a concentration between 0.1 mg/ml and 5,000 mg/ml;
poloxamer F108, in particular at a concentration between 0.1 mg/ml and 5,000 mg/ml;
Silibinin, in particular at a concentration between 0.05 μM and 500 μM;
Midostaurin, in particular at a concentration between 2 nM and 500,000 nM;
PEG-PLA-PEG, in particular at a concentration between 1 μg/ml and 5,000 μg/ml;
PEG-PGLA-PEG, in particular at a concentration between 1 μg/ml and 5,000 μg/ml;
PEG-PCL-PEG, in particular at a concentration between 1 μg/ml and 5,000 μg/ml;
Nystatin, in particular at a concentration between 0.1 and 1000 μM;
Natamycin, in particular at a concentration between 0.05 and 500 μM;
Ruxolitinib, in particular at a concentration between 0.01 and 10,000 μM;
Fludarabine, in particular at a concentration between 0.01 and 10,000 μM;
Everolimus, in particular at a concentration between 0.1 and 10 μM;
Resveratrol, in particular at a concentration between 0.1 and 25 μM;
Prostaglandin E, in particular at a concentration between 1 and 100 μM;
Desoxyribonucleosides, in particular at a concentration between 0.1 and 10 each nucleoside; and/or
any combination thereof.

In addition, it has to be noted that both Amphotericin B and the compounds listed above may be combined at any of the concentrations disclosed herein.

In a particular embodiment, the invention relates to the method according to the invention, wherein Amphotericin B is combined with the transduction enhancer DMSO, in particular at a concentration between 0.1 and 10% (v/v); and optionally in combination with one or more of the compounds listed above at any of the concentrations listed above.

In a particular embodiment, the invention relates to the method according to the invention, wherein at least one additional transduction enhancing compound is selected from the group consisting of: Lentiboost®, poloxamer F108 and/or a PEG-PCL-PEG polymer.

Further, the inventors have surprisingly found that Silibinin can be used as a transduction enhancer, which has not been suggested before. Thus, in a particular embodiment, the invention relates to the method according to the invention, wherein the transduction enhancing compound is Silibinin.

Silibinin may be contacted with the target cell to induce transduction efficiency with a retroviral vector during the pre- and/or co-incubation step at any of the concentrations disclosed herein. Thus, in a particular embodiment, the invention relates to the method according to the invention, wherein the target cell is contacted with Silibinin during the pre-incubation and/or co-incubation step at a concentration of about 0.05 to about 500 μM, or any of the concentrations disclosed herein. Further, Silibinin may be combined with any transduction enhancing compound known in the art or any of the transduction enhancing compounds described herein, preferably at any of the concentrations disclosed herein. Thus, in a particular embodiment, the invention relates to the method according to the invention, wherein Silibinin is used in combination with one or more additional transduction enhancing compounds.

In a particular embodiment, the invention relates to the according to the invention, wherein the one or more additional transduction enhancing compound used in combination with Silibinin is selected from the group consisting of:
Lentiboost®, in particular at a concentration between 0.1 mg/ml and 5,000 mg/ml;
poloxamer F108, in particular at a concentration between 0.1 μg/ml and 5,000 mg/ml;
a protamine salt, in particular at a concentration between 0.05 and 25 μg/mL;
Amphotericin B, in particular at a concentration between 0.05 μM and 500 μM;
Midostaurin, in particular at a concentration between 2 and 500,000 nM;
a PEG-PLA-PEG polymer, in particular at a concentration between 1 μg/ml and 5,000 μg/ml;
a PEG-PGLA-PEG polymer, in particular at a concentration between 1 μg/ml and 5,000 μg/ml;
a PEG-PCL-PEG polymer, in particular at a concentration between 1 μg/ml and 5,000 μg/ml;
Nystatin, in particular at a concentration between 0.1 and 1000 μM;
Natamycin, in particular at a concentration between 0.05 and 500 μM;
Ruxolitinib, in particular at a concentration between 0.01 and 10,000 μM;
Fludarabine, in particular at a concentration between 0.01 and 10,000 μM;
Everolimus, in particular at a concentration between 0.1 and 10 μM;
Resveratrol, in particular at a concentration between 0.1 and 25 μM;
Prostaglandin E, in particular at a concentration between 1 and 100 μM;
Desoxyribonucleosides, in particular at a concentration between 0.1 mM and 10 mM of each nucleoside; and/or
any combination thereof.

It has to be noted that both Silibinin and the compounds listed above may be combined at any of the concentrations disclosed herein.

In a particular embodiment, the invention relates to the method according to the invention, wherein Silibinin is combined with the transduction enhancer DMSO, in particular at a concentration between 0.1 and 10% (v/v); and optionally in combination with one or more of the compounds listed above at any of the concentrations listed above.

Preferably, Silibinin may be combined with Lentiboost®, poloxamer F108 or a PEG-PCL-PEG polymer at any of the concentrations disclosed herein.

Further, the inventors have surprisingly found that Midostaurin can be used as a transduction enhancer, which has not been suggested before. Thus, in a particular embodiment, the invention relates to the method according to the invention, wherein the transduction enhancing compound is Midostaurin.

Midostaurin may be contacted with the target cell to induce transduction efficiency with a retroviral vector during the pre- and/or co-incubation step at any of the concentrations disclosed herein. Thus, in a particular embodiment, the invention relates to the method according to the invention, wherein the target cell is contacted with Midostaurin during the pre-incubation and/or co-incubation step at a concentration between 2 nM and 500,000 nM, or any of the concentrations disclosed herein. Further, Midostaurin may be combined with any transduction enhancing compound known in the art or any of the transduction enhancing compounds described herein, preferably at any of the concentrations disclosed herein. Thus, in a particular embodiment, the invention relates to the method according to the invention, wherein Midostaurin is used in combination with one or more additional transduction enhancing compounds.

In a particular embodiment, the invention relates to the according to the invention, wherein the one or more additional transduction enhancing compound used in combination with Midostaurin is selected from the group consisting of:
Lentiboost®, in particular at a concentration between 0.1 mg/ml and 5,000 mg/ml;
poloxamer F108, in particular at a concentration between 0.1 mg/ml and 5,000 mg/ml;
a protamine salt, in particular at a concentration between 0.05 and 25 μg/mL;
Amphotericin B, in particular at a concentration between 0.05 μM and 500 μM;
Silibinin, in particular at a concentration between 0.05 μM and 500 μM;
a PEG-PLA-PEG polymer, in particular at a concentration between 1 μg/ml and 5,000 μg/ml;
a PEG-PGLA-PEG polymer, in particular at a concentration between 1 μg/ml and 5,000 μg/ml;
a PEG-PCL-PEG polymer, in particular at a concentration between 1 ng/ml and 5,000 ƒg/ml;
Nystatin, in particular at a concentration between 0.1 and 1000 μM;
Natamycin, in particular at a concentration between 0.05 and 500 μM;
Ruxolitinib, in particular at a concentration between 0.01 and 10,000 μM;
Fludarabine, in particular at a concentration between 0.01 and 10,000 μM;
Everolimus, in particular at a concentration between 0.1 and 10 μM;
Resveratrol, in particular at a concentration between 0.1 and 25 μM;
Prostaglandin E, in particular at a concentration between 1 and 100 μM;
Desoxyribonucleosides, in particular at a concentration between 0.1 mM and 10 mM of each nucleoside; and/or
any combination thereof.

It has to be noted that both Midostaurin and the compounds listed above may be combined at any of the concentrations disclosed herein.

In a particular embodiment, the invention relates to the method according to the invention, wherein Midostaurin is combined with the transduction enhancer DMSO, in particular at a concentration between 0.1 and 10% (v/v); and optionally in combination with one or more of the compounds listed above at any of the concentrations listed above.

Preferably, Midostaurin may be combined with Lentiboost® or poloxamer F108 at any of the concentrations disclosed herein.

Further, the inventors have surprisingly found that Nystatin can be used as a transduction enhancer, which has not been suggested before. Thus, in a particular embodiment, the invention relates to the method according to the invention, wherein the transduction enhancing compound is Nystatin.

Nystatin may be contacted with the target cell to induce transduction efficiency with a retroviral vector during the pre- and/or co-incubation step at any of the concentrations disclosed herein. Thus, in a particular embodiment, the invention relates to the method according to the invention, wherein the target cell is contacted with Nystatin during the pre-incubation and/or co-incubation step at a concentration between 0.1 and 1000 μM, or any of the concentrations disclosed herein. Further, Nystatin may be combined with any transduction enhancing compound known in the art or any of the transduction enhancing compounds described herein, preferably at any of the concentrations disclosed herein. Thus, in a particular embodiment, the invention relates to the method according to the invention, wherein Nystatin is used in combination with one or more additional transduction enhancing compounds.

In a particular embodiment, the invention relates to the according to the invention, wherein the one or more additional transduction enhancing compound used in combination with Nystatin is selected from the group consisting of:
Lentiboost®, in particular at a concentration between 0.1 mg/ml and 5,000 mg/ml;
poloxamer F108, in particular at a concentration between 0.1 mg/ml and 5,000 mg/ml;
a protamine salt, in particular at a concentration between 0.05 and 25 μg/mL;
Amphotericin B, in particular at a concentration between 0.05 μM and 500 μM;
Silibinin, in particular at a concentration between 0.05 μM and 500 μM;
a PEG-PLA-PEG polymer, in particular at a concentration between 1 μg/ml and 5,000 μg/ml;
a PEG-PGLA-PEG polymer, in particular at a concentration between 1 μg/ml and 5,000 μg/ml;
a PEG-PCL-PEG polymer, in particular at a concentration between 1 μg/ml and 5,000 μg/ml;
Midostaurin, in particular at a concentration between 2 nM and 500,000 nM;
Natamycin, in particular at a concentration between 0.05 and 500 μM;
Ruxolitinib, in particular at a concentration between 0.01 and 10,000 μM;
Fludarabine, in particular at a concentration between 0.01 and 10,000 μM;
Everolimus, in particular at a concentration between 0.1 and 10 μM;
Resveratrol, in particular at a concentration between 0.1 and 25 μM;
Prostaglandin E, in particular at a concentration between 1 and 100 μM;
Desoxyribonucleosides, in particular at a concentration between 0.1 and 10 mM of each nucleoside; and/or
any combination thereof.

It has to be noted that both Nystatin and the compounds listed above may be combined at any of the concentrations disclosed herein.

In a particular embodiment, the invention relates to the method according to the invention, wherein Nystatin is combined with the transduction enhancer DMSO, in particular at a concentration between 0.1 and 10% (v/v); and optionally in combination with one or more of the compounds listed above at any of the concentrations listed above.

Further, the inventors have surprisingly found that Natamycin can be used as a transduction enhancer, which has not been suggested before. Thus, in a particular embodiment, the invention relates to the method according to the invention, wherein the transduction enhancing compound is Natamycin.

Natamycin may be contacted with the target cell to induce transduction efficiency with a retroviral vector during the pre- and/or co-incubation step at any of the concentrations disclosed herein. Thus, in a particular embodiment, the invention relates to the method according to the invention, wherein the target cell is contacted with Natamycin during the pre-incubation and/or co-incubation step at a concentration between 0.05 and 500 μM, or any of the concentrations disclosed herein. Further, Natamycin may be combined with any transduction enhancing compound known in the art or any of the transduction enhancing compounds described herein, preferably at any of the concentrations disclosed herein. Thus, in a particular embodiment, the invention relates to the method according to the invention, wherein Natamycin is used in combination with one or more additional transduction enhancing compounds.

In a particular embodiment, the invention relates to the according to the invention, wherein the one or more additional transduction enhancing compound used in combination with Natamycin is selected from the group consisting of:
Lentiboost®, in particular at a concentration between 0.1 mg/ml and 5,000 mg/ml;
poloxamer F108, in particular at a concentration between 0.1 mg/ml and 5,000 mg/ml;
a protamine salt, in particular at a concentration between 0.05 and 25 μg/mL;
Amphotericin B, in particular at a concentration between 0.05 μM and 500 μM;
Silibinin, in particular at a concentration between 0.05 μM and 500 μM;
a PEG-PLA-PEG polymer, in particular at a concentration between 1 μg/ml and 5,000 μg/ml;
a PEG-PGLA-PEG polymer, in particular at a concentration between 1 μg/ml and 5,000 μg/ml;
a PEG-PCL-PEG polymer, in particular at a concentration between 1 μg/ml and 5,000
Midostaurin, in particular at a concentration between 2 nM and 500,000 nM;
Nystatin, in particular at a concentration between 0.1 and 1000 μM;
Ruxolitinib, in particular at a concentration between 0.01 and 10,000 μM;
Fludarabine, in particular at a concentration between 0.01 and 10,000 μM;
Everolimus, in particular at a concentration between 0.1 and 10 μM;
Resveratrol, in particular at a concentration between 0.1 and 25 μM;
Prostaglandin E, in particular at a concentration between 1 and 100 μM;
Desoxyribonucleosides, in particular at a concentration between 0.1 mM and 10 mM of each nucleoside; and/or
any combination thereof.

It has to be noted that both Natamycin and the compounds listed above may be combined at any of the concentrations disclosed herein.

In a particular embodiment, the invention relates to the method according to the invention, wherein Natamyin is combined with the transduction enhancer DMSO, in particular at a concentration between 0.1 and 10% (v/v); and optionally in combination with one or more of the compounds listed above at any of the concentrations listed above.

Further, the inventors have surprisingly found that Fludarabine may be used as a transduction enhancer, which has not been suggested before. Thus, in a particular embodiment, the invention relates to the method according to the invention, wherein the transduction enhancing compound is Fludarabine.

Fludarabine may be contacted with the target cell to induce transduction efficiency with a retroviral vector during the pre- and/or co-incubation step at any of the concentrations disclosed herein. Thus, in a particular embodiment, the invention relates to the method according to the invention, wherein the target cell is contacted with Fludarabine during the pre-incubation and/or co-incubation step at a concentration between 0.01 and 10,000 μM, or any of the concentrations disclosed herein. Further, Fludarabine may be combined with any transduction enhancing compound known in the art or any of the transduction enhancing compounds described herein, preferably at any of the concentrations disclosed herein. Thus, in a particular embodiment, the invention relates to the method according to the invention, wherein Fludarabine is used in combination with one or more additional transduction enhancing compounds.

In a particular embodiment, the invention relates to the according to the invention, wherein the one or more additional transduction enhancing compound used in combination with Fludarabine is selected from the group consisting of:
Lentiboost® in particular at a concentration between 0.1 mg/ml and 5,000 mg/ml;
poloxamer F108, in particular at a concentration between 0.1 mg/ml and 5,000 mg/ml;
a protamine salt, in particular at a concentration between 0.05 and 25 μg/mL;
Amphotericin B, in particular at a concentration between 0.05 μM and 500 μM; Silibinin, in particular at a concentration between 0.05 μM and 500 μM;
a PEG-PLA-PEG polymer, in particular at a concentration between 1 μg/ml and 5,000 μg/ml;
a PEG-PGLA-PEG polymer, in particular at a concentration between 1 μg/ml and 5,000 μg/ml;
a PEG-PCL-PEG polymer, in particular at a concentration between 1 μg/ml and 5,000 μg/ml;
Nystatin, in particular at a concentration between 0.1 and 1000 μM;
Natamycin, in particular at a concentration between 0.05 and 500 μM;
Ruxolitinib, in particular at a concentration between 0.01 and 10,000 μM;
Midostaurin, in particular at a concentration between 2 and 500,000
Everolimus, in particular at a concentration between 0.1 and 10 μM;
Resveratrol, in particular at a concentration between 0.1 and 25 μM;
Prostaglandin E, in particular at a concentration between 1 and 100 μM;
Desoxyribonucleosides, in particular at a concentration between 0.1 mM and 10 mM of each nucleoside; and/or
any combination thereof.

It has to be noted that both Fludarabine and the compounds listed above may be combined at any of the concentrations disclosed herein.

In a particular embodiment, the invention relates to the method according to the invention, wherein Fludarabine is combined with the transduction enhancer DMSO, in particular at a concentration between 0.1 and 10% (v/v); and optionally in combination with one or more of the compounds listed above at any of the concentrations listed above.

Further, the inventors have surprisingly found that Ruxolitinib may be used as a transduction enhancer, which has not been suggested before. Thus, in a particular embodiment, the invention relates to the method according to the invention, wherein the transduction enhancing compound is Ruxolitinib.

Ruxolitinib may be contacted with the target cell to induce transduction efficiency with a retroviral vector during the pre- and/or co-incubation step at any of the concentrations disclosed herein. Thus, in a particular embodiment, the invention relates to the method according to the invention, wherein the target cell is contacted with Ruxolitinib during the pre-incubation and/or co-incubation step at a concentration between 0.01 and 10,000 μM, or any of the concentrations disclosed herein. Further, Ruxolitinib may be combined with any transduction enhancing compound known in the art or any of the transduction enhancing compounds described herein, preferably at any of the concentrations disclosed herein. Thus, in a particular embodiment, the invention relates to the method according to the invention, wherein Ruxolitinib is used in combination with one or more additional transduction enhancing compounds.

In a particular embodiment, the invention relates to the according to the invention, wherein the one or more additional transduction enhancing compound used in combination with Ruxolitinib is selected from the group consisting of:
Lentiboost®, in particular at a concentration between 0.1 mg/ml and 5,000 mg/ml;
poloxamer F108, in particular at a concentration between 0.1 mg/ml and 5,000 mg/ml;
a protamine salt, in particular at a concentration between 0.05 and 25 μg/mL;
Amphotericin B, in particular at a concentration between 0.05 μM and 500 μM;
Silibinin, in particular at a concentration between 0.05 μM and 500 μM;
a PEG-PLA-PEG polymer, in particular at a concentration between 1 μg/ml and 5,000 μg/ml;
a PEG-PGLA-PEG polymer, in particular at a concentration between 1 μg/ml and 5,000 μg/ml;
a PEG-PCL-PEG polymer, in particular at a concentration between 1 μg/ml and 5,000 μg/ml;
Midostaurin, in particular at a concentration between 2 nM and 500,000 nM;
Natamycin, in particular at a concentration between 0.05 and 500 μM;
Nystatin, in particular at a concentration between 0.1 and 1000 μM;
Fludarabine, in particular at a concentration between 0.01 and 10,000 μM;
Everolimus, in particular at a concentration between 0.1 and 10 μM;
Resveratrol, in particular at a concentration between 0.1 and 25 μM;
Prostaglandin E, in particular at a concentration between 1 and 100 μM;
Desoxyribonucleosides, in particular at a concentration between 0.1 mM and 10 mM of each nucleoside; and/or
any combination thereof.

It has to be noted that both Ruxolitinib and the compounds listed above may be combined at any of the concentrations disclosed herein.

In a particular embodiment, the invention relates to the method according to the invention, wherein Ruxolitinib is combined with the transduction enhancer DMSO, in particular at a concentration between 0.1 and 10% (v/v); and optionally in combination with one or more of the compounds listed above at any of the concentrations listed above.

Further, the inventors have surprisingly found that PEG-PCL-PEG polymers can be used as a transduction enhancer, which has not been suggested before. Thus, in a particular embodiment, the invention relates to the method according to the invention, wherein the transduction enhancing compound is a PEG-PCL-PEG polymer as disclosed herein.

PEG-PCL-PEG polymers may be contacted with the target cell to induce transduction efficiency with a retroviral vector during the pre- and/or co-incubation step at any of the concentrations disclosed herein. Thus, in a particular embodiment, the invention relates to the method according to the invention, wherein the target cell is contacted with a PEG-PCL-PEG polymer during the pre-incubation and/or co-incubation step at a concentration between 1 μg/ml and 5,000 μg/ml, or any of the concentrations disclosed herein. Peg, a PEG-PCL-PEG polymer may be combined with any transduction enhancing compound known in the art or any of the transduction enhancing compounds described herein, preferably at any of the concentrations disclosed herein. Thus, in a particular embodiment, the invention relates to the method according to the invention, wherein a PEG-PCL-PEG polymer is used in combination with one or more additional transduction enhancing compounds.

In a particular embodiment, the invention relates to the according to the invention, wherein the one or more additional transduction enhancing compound used in combination with a PEG-PCL-PEG polymer is selected from the group consisting of:
Lentiboost®, in particular at a concentration between 0.1 mg/ml and 5,000 mg/ml;
poloxamer F108, in particular at a concentration between 0.1 mg/ml and 5,000 mg/ml;
a protamine salt, in particular at a concentration between 0.05 and 25 μg/mL;
Amphotericin B, in particular at a concentration between 0.05 μM and 500 μM;
Silibinin, in particular at a concentration between 0.05 μM and 500
a PEG-PLA-PEG polymer, in particular at a concentration between 1 μg/ml and 5,000 μg/ml;
a PEG-PLGA-PEG polymer, in particular at a concentration between 1 μg/ml and 5,000 μg/ml;
Natamycin, in particular at a concentration between 0.05 and 500 μM;
Midostaurin, in particular at a concentration between 2 and 500,000 nM;
Nystatin, in particular at a concentration between 0.1 and 1000 μM;
Ruxolitinib, in particular at a concentration between 0.01 and 10,000 μM;
Fludarabine, in particular at a concentration between 0.01 and 10,000 μM;
Everolimus, in particular at a concentration between 0.1 and 10 μM;
Resveratrol, in particular at a concentration between 0.1 and 25 μM;
Prostaglandin E, in particular at a concentration between 1 and 100 μM;
Desoxyribonucleosides, in particular at a concentration between 0.1 and 10 mM of each nucleoside; and/or
any combination thereof.

It has to be noted that both the PEG-PCL-PEG polymer and the compounds listed above may be combined at any of the concentrations disclosed herein.

In a particular embodiment, the invention relates to the method according to the invention, wherein PEG-PCL-PEG is combined with the transduction enhancer DMSO, in particular at a concentration between 0.1 and 10% (v/v); and optionally in combination with one or more of the compounds listed above at any of the concentrations listed above.

Preferably, the PEG-PCL-PEG polymer may be combined with a protamine salt or Silibinin at any of the concentrations disclosed herein.

Further, the inventors have surprisingly found that PEG-PLGA-PEG polymers can be used as a transduction enhancer, which has not been suggested before. Thus, in a particular embodiment, the invention relates to the method according to the invention, wherein the transduction enhancing compound is a PEG-PLGA-PEG polymer as disclosed herein.

PEG-PLGA-PEG polymers may be contacted with the target cell to induce transduction efficiency with a retroviral vector during the pre- and/or co-incubation step at any of the concentrations disclosed herein. Thus, in a particular embodiment, the invention relates to the method according to the invention, wherein the target cell is contacted with a PEG-PLGA-PEG polymer during the pre-incubation and/or co-incubation step at a concentration between 1 μg/ml and 5,000 μg/ml, or any of the concentrations disclosed herein. Per, a PEG-PLGA-PEG polymer may be combined with any transduction enhancing compound known in the art or any of the transduction enhancing compounds described herein, preferably at any of the concentrations disclosed herein. Thus, in a particular embodiment, the invention relates to the method according to the invention, wherein a PEG-PLGA-PEG polymer is used in combination with one or more additional transduction enhancing compounds.

In a particular embodiment, the invention relates to the according to the invention, wherein the one or more additional transduction enhancing compound used in combination with a PEG-PLGA-PEG polymer is selected from the group consisting of:
Lentiboost®, in particular at a concentration between 0.1 mg/ml and 5,000 mg/ml;
poloxamer F108, in particular at a concentration between 0.1 mg/ml and 5,000 mg/ml;
a protamine salt, in particular at a concentration between 0.05 and 25 μg/mL;
Amphotericin B, in particular at a concentration between 0.05 M and 500 μM;
Silibinin, in particular at a concentration between 0.05 μM and 500 μM;
a PEG-PLA-PEG polymer, in particular at a concentration between 1 μg/ml and 5,000 μg/ml;
a PEG-PCL-PEG polymer, in particular at a concentration between 1 μg/ml and 5,000 μg/ml;
Natamycin, in particular at a concentration between 0.05 and 500 μM;
Midostaurin, in particular at a concentration between 2 nM and 500,000 nM;
Nystatin, in particular at a concentration between 0.1 and 1000 μM;
Ruxolitinib, in particular at a concentration between 0.01 and 10,000 μM;
Fludarabine, in particular at a concentration between 0.01 and 10,000 μM;
Everolimus, in particular at a concentration between 0.1 and 10 μM;
Resveratrol, in particular at a concentration between 0.1 and 25 μM
Prostaglandin E, in particular at a concentration between 1 and 100 μM;
Desoxyribonucleosides, in particular at a concentration between 0.1 and 10 mM each nucleoside; and/or
any combination thereof.

It has to be noted that both the PEG-PLGA-PEG polymer and the compounds listed above may be combined at any of the concentrations disclosed herein.

In a particular embodiment, the invention relates to the method according to the invention, wherein PEG-PLGA-PEG is combined with the transduction enhancer DMSO, in particular at a concentration between 0.1 and 10% (v/v); and optionally in combination with one or more of the compounds listed above at any of the concentrations listed above.

Further, the inventors have surprisingly found that PEG-PLA-PEG polymers can be used as a transduction enhancer, which has not been suggested before. Thus, in a particular embodiment, the invention relates to the method according to the invention, wherein the transduction enhancing compound is a PEG-PLA-PEG polymer as disclosed herein.

PEG-PLA-PEG polymers may be contacted with the target cell to induce transduction efficiency with a retroviral vector during the pre- and/or co-incubation step at any of the concentrations disclosed herein. Thus, in a particular embodiment, the invention relates to the method according to the invention, wherein the target cell is contacted with a PEG-PLA-PEG polymer during the pre-incubation and/or co-incubation step at a concentration between 1 μg/ml and 5,000 μg/ml, or any of the concentrations disclosed herein. Further, a PEG-PLA-PEG polymer may be combined with any transduction enhancing compound known in the art or any of the transduction enhancing compounds described herein, preferably at any of the concentrations disclosed herein. Thus, in a particular embodiment, the invention relates to the method according to the invention, wherein a PEG-PLA-PEG polymer is used in combination with one or more additional transduction enhancing compounds.

In a particular embodiment, the invention relates to the according to the invention, wherein the one or more additional transduction enhancing compound used in combination with a PEG-PLA-PEG polymer is selected from the group consisting of:
Lentiboost®, in particular at a concentration between 0.1 mg/ml and 5,000 mg/ml;
poloxamer F108, in particular at a concentration between 0.1 mg/ml and 5,000 mg/ml;
a protamine salt, in particular at a concentration between 0.05 and 25 μg/mL;
Amphotericin B, in particular at a concentration between 0.05 μM and 500 μM;
Silibinin, in particular at a concentration between 0.05 μM and 500
a PEG-PLGA-PEG polymer, in particular at a concentration between 1 μg/ml and 5,000 μg/ml;
a PEG-PCL-PEG polymer, in particular at a concentration between 1 μg/ml and 5,000 μg/ml;
Natamycin, in particular at a concentration between 0.05 and 500 μM;
Midostaurin, in particular at a concentration between 2 and 500,000 nM;
Nystatin, in particular at a concentration between 0.1 and 1000 μM;
Ruxolitinib, in particular at a concentration between 0.01 and 10,000 μM;
Fludarabine, in particular at a concentration between 0.01 and 10,000 μM;
Everolimus, in particular at a concentration between 0.1 and 10 μM;
Resveratrol, in particular at a concentration between 0.1 and 25 μM;
Prostaglandin E, in particular at a concentration between 1 and 100 μM;
Desoxyribonucleosides, in particular at a concentration between 0.1 and 10 of each nucleoside; and/or
any combination thereof.

It has to be noted that both the PEG-PLA-PEG polymer and the compounds listed above may be combined at any of the concentrations disclosed herein.

In a particular embodiment, the invention relates to the method according to the invention, wherein PEG-PLA-PEG is combined with the transduction enhancer DMSO, in particular at a concentration between 0.1 and 10% (v/v); and optionally in combination with one or more of the compounds listed above at any of the concentrations listed above.

It is to be understood that all compounds and combinations of compounds listed in the embodiments above are disclosed herein in any of the concentrations or concentration ranges disclosed elsewhere herein.

### BRIEF DESCRIPTION OF THE FIGURES

FIG. 1 summarizes the results of three independent experiments, in which human CD34-positive HSC were transduced with a lentiviral self-inactivating gene therapy vector. This vector encodes human p47phox cDNA under control of the miR223 internal promoter. Cells were transduced in presence of protamine sulfate only (resulting VCN set to 100%), or in presence of protamine sulfate (PS) plus one or more compounds tested for transduction enhancer activity. After 10 to 12 days post transduction, DNA from transduced cells was isolated, and VCNs were quantified by qPCR. The increase in VCN relative to the VCN achieved upon transduction in presence of protamine sulfate only was expressed as “fold induction rel. to PS”. Left panel: Transduction at an MOI of 1. Right panel. Transduction at an MOI of 3. PGE2: prostaglandin E2; AmphoB: Amphotericin B.

FIG. 2 summarizes the results of three independent experiments, in which human CD34-positive HSC were transduced with a lentiviral self-inactivating gene therapy vector. Cells were transduced in X-Vivo 10 medium either in the absence of a transduction enhancer or in the presence of compounds that are tested for transduction enhancer activity. After 10 to 12 days post transduction, DNA from transduced cells was isolated, and VCNs were quantified by qPCR. The solid line represents the average VCN obtained with Lentiboost at the recommended concentration of 1 mg/mL. PCL: PEG-PCL-PEG (14.2 kDa); PLA: PEG-PLA-PEG (14.2 kDa).

FIG. 3 summarizes the results of three independent experiments, in which human CD34-positive HSC were transduced with a lentiviral self-inactivating gene therapy vector. Cells were transduced in BESP1366F medium either in the absence of a transduction enhancer or in the presence of compounds that are tested for transduction enhancer activity. After 10 to 12 days post transduction, DNA from transduced cells was isolated, and VCNs were quantified by qPCR. The solid line represents the average VCN obtained with Lentiboost® at the recommended concentration of 1 mg/mL.

FIG. 4 summarizes the results of three independent experiments, in which human CD34-positive HSC were transduced with a lentiviral self-inactivating gene therapy vector. Cells were transduced in BESP1366F medium in the presence of protamine, Amphotericin B or a combination thereof. After 10 to 12 days post transduction, DNA from transduced cells was isolated, and VCNs were quantified by qPCR. The solid line represents the average VCN obtained with Lentiboost® at the recommended concentration of 1 mg/mL under comparable conditions (see solid line in FIG. 3). The dashed line represents the average VCN obtained with protamine alone.

FIG. 5 summarizes the results of three independent experiments, in which human CD34-positive HSC were transduced with a lentiviral self-inactivating gene therapy vector. Cells were transduced in BESP1366F medium in the presence of Lentiboost®, Amphotericin B, Silibinin, Midostaurin or combinations thereof comprising Lentiboost® at a concentration of 1 mg/mL. After 10 to 12 days post transduction, DNA from transduced cells was isolated, and VCNs were quantified by qPCR. The solid line represents the average VCN obtained with Lentiboost® at the recommended concentration of 1 mg/mL.

FIG. 6 summarizes the results of three independent experiments, in which human CD34-positive HSC were transduced with a lentiviral self-inactivating gene therapy vector. Cells were transduced in X-Vivo 10 medium in the presence of protamine, PEG-PCL-PEG (14.2 kDa), poloxamer F108 or combinations thereof comprising protamine. After 10 to 12 days post transduction, DNA from transduced cells was isolated, and VCNs were quantified by qPCR. The solid line represents the average VCN obtained with Lentiboost® at the recommended concentration of 1 mg/mL under comparable conditions (see FIG. 2). The dashed line represents the average VCN obtained with protamine alone.

FIG. 7 summarizes the results of three independent experiments, in which human CD34-positive HSC were transduced with a lentiviral self-inactivating gene therapy vector. Cells were transduced in X-Vivo 10 medium in the presence of Lentiboost®, Amphotericin B, protamine, Silibinin, Midostaurin or combinations thereof comprising Lentiboost. After 10 to 12 days post transduction, DNA from transduced cells was isolated, and VCNs were quantified by qPCR. The solid line represents the average VCN obtained with Lentiboost® at the recommended concentration of 1 mg/mL (see FIG. 2).

FIG. 8 summarizes the results of three independent experiments, in which human CD34-positive HSC were transduced with a lentiviral self-inactivating gene therapy vector. Cells were transduced in X-Vivo 10 medium in the presence of Amphotericin B, Lentiboost®, poloxamer F108, PEG-PCL-PEG (14.2 kDa) or combinations thereof comprising Amphotericin B. After 10 to 12 days post transduction, DNA from transduced cells was isolated, and VCNs were quantified by qPCR. The solid line represents the average VCN obtained with Lentiboost® at the recommended concentration of 1 mg/mL under comparable conditions. The dashed line represents the average VCN obtained with Amphotericin B alone.

FIG. 9 summarizes the results of three independent experiments, in which human CD34-positive HSC were transduced with a lentiviral self-inactivating gene therapy vector. Cells were transduced in X-Vivo 10 medium in the presence of Silibinin, Lentiboost®, PEG-PCL-PEG (14.2 kDa), poloxamer F108 or combinations thereof comprising Silibinin. After 10 to 12 days post transduction, DNA from transduced cells was isolated, and VCNs were quantified by qPCR. The solid line represents the average VCN obtained with Lentiboost® at the recommended concentration of 1 mg/mL under comparable conditions. The dashed line represents the average VCN obtained with Silibinin alone.

FIG. 10 summarizes the results of three independent experiments, in which human CD34-positive HSC were transduced with a lentiviral self-inactivating gene therapy vector. Cells were transduced in X-Vivo 10 medium in the presence of Midostaurin, Lentiboost®, poloxamer F108, PEG-PCL-PEG (14.2 kDa) or combinations thereof comprising Midostaurin. After 10 to 12 days post transduction, DNA from transduced cells was isolated, and VCNs were quantified by qPCR. The solid line represents the average VCN obtained with Lentiboost® at the recommended concentration of 1 mg/mL under comparable conditions. The dashed line represents the average VCN obtained with Midostaurin alone.

FIG. 11 summarizes the results of three independent experiments, in which human CD34-positive HSC were transduced with a lentiviral self-inactivating gene therapy vector. Cells were transduced in X-Vivo 10 medium in the presence of PEG-PCL-PEG (14.2 kDa), protamine, Amphotericin B, Silibinin, Midostaurin or combinations thereof comprising PEG-PCL-PEG. After 10 to 12 days post transduction, DNA from transduced cells was isolated, and VCNs were quantified by qPCR. The solid line represents the average VCN obtained with Lentiboost® at the recommended concentration of 1 mg/mL under comparable conditions (see FIG. 2). The dashed line represents the average VCN obtained with PEG-PCL-PEG alone.

FIG. 12 summarizes the results of three independent experiments, in which human CD34-positive HSC were transduced with a lentiviral self-inactivating gene therapy vector. Cells were transduced in X-Vivo 10 medium in the presence of poloxamer F108, Silibinin, Midostaurin, Amphotericin B, protamine or combinations thereof comprising poloxamer F108. After 10 to 12 days post transduction, DNA from transduced cells was isolated, and

VCNs were quantified by qPCR. The solid line represents the average VCN obtained with Lentiboost® at the recommended concentration of 1 mg/mL under comparable conditions (see FIG. 2). The dashed line represents the average VCN obtained with poloxamer F108 alone.

FIG. 13 summarizes the results of three independent experiments, in which human CD34-positive HSC were transduced with a lentiviral self-inactivating gene therapy vector at MOI 20. Cells were transduced in the absence and presence of 1% DMSO.

### EXAMPLES

### Example 1: Transduction at MOI 1, MOI 3 or MOI 5

Thawed cells from healthy donors were cultured in 96 well plates comprising X-Vivo 20 medium supplemented with 1% human serum albumin, 300 μg/ml stem cell factor (SCF), 300 μg/ml fins like tyrosine kinase 3 (FLT-3) ligand (Flt3-lig) and 100 μng/ml Thrombopoietin (TPO) at a density of 0.5 E6 cells/cm², and at a concentration of 1E6 cells/ml for 22h, optionally followed by 2h of pre-stimulation with below mentioned compounds. Afterwards, cells were incubated for transduction with the lentiviral SIN gene therapy vector for 12 h in presence of the compounds indicated below and protamine sulfate. After the 12 h transduction period, the medium was exchanged by above mentioned medium, and cells were transferred to 12-well plates in which they were cultured in a volume of 1 ml for 5 to 7 days depending on cell density. Thereafter, medium was exchanged by fresh medium, and cells were cultured in 2 ml for another 6 days. At day 12 post transduction, DNA was isolated, and vector copy number (VCN) was quantified by qPCR. All experiments were conducted in triplicates. For negative control, a triplicate of non-transduced cells was cultured as described. To determine the baseline of transduction efficacy in presence of only protamine sulfate, cells were incubated with MOI 1, MOI 3 or MOI 5 of the lentiviral SIN vector in presence of 4 μg/mL protamine sulfate. For transduction in the presence of potential transduction enhancers, cells were incubated with MOI 1, MOI 3 or MOI 5 of the lentiviral SIN vector in presence of
i) 5 μM Silibinin, 4 μg/mL protamine sulfate;
ii) 5 μM Resveratrol, 4 μg/mL protamine sulfate;
iii) 1 μM Everolimus, 4 μg/mL protamine sulfate;
iv) 0,4 μM Midostaurin, 4 μg/mL protamine sulfate;
v) 1 μg/mL Amphotericin B, 4 μg/mL protamine sulfate;
vi) 100 μM Nystatin, 4 μg/mL protamine sulfate;
vii) 3 μM Natamycin, 4 μg/mL protamine sulfate;
viii) 10 μM Prostaglandin E2, 4 μg/mL protamine sulfate;
ix) 1 mg/ml Lentiboost®, 4 μg/mL protamine sulfate;
x) 1,000 μg/ml Poloxamer Symperonic F108®, 4 μg/mL protamine sulfate;
xi) 1,000 μg/ml poly(ethylene glycol)-b-poly(D,L-lactic acid-co-glycolic acid)-b-poly(ethylene glycol) (PEG- PLGA-PEG) with 5 kDa poly(ethylene glycol) block on both ends, and a central 4.2 kDa poly(D,L-lactic acid-co-glycolic acid) block, termed PEG5k-b-PLA4.2k-b-PEG5k;
xii) 10 μg/ml methoxypoly(ethylene glycol)-poly(e-caprolacton)-methoxypoly(ethylene glycol) (PEG- PCL-PEG) with 5 kDa poly(ethylene glycol) block on both ends, and a central 4.2 kDa poly(e- caprolacton) block, termed PEG5k-b-PCL4.2k-b-PEG5k;
xiii) 50 μg/mL poly(ethylene glycol)/poly(lactide)/poly(ethylene glycol) (PEG-PLA-PEG) with 5 kDa poly(ethylene glycol) blocks on both ends, and a central 4.2 kDa poly(lactide) block, termed PEG5k-b-PLA4.2k-b-PEG5k
xiv) 50 μg/ml methoxypoly(ethylene glycol)-poly(e-caprolacton)-methoxypoly(ethylene glycol) (PEG- PCL-PEG) with a central 2.4 kDa poly(e-caprolacton) block, and a 5.3 kDa poly(ethylene glycol) block on both ends, which both were covalently linked to an amino group, termed 2-PEG5.3k-b- PCL2.4k-b-PEG5.3k-NH2;
xv) Deoxyribonucleosides with a final concentration of 300 μM of each, or with combinations thereof.

After 10 to 12 days, non-integrated pro-viral DNA was diluted out due to the proliferation of cells in the plate and VCNs were quantified thereafter by qPCR. Every condition was tested in triplicates and the average of VCNs of triplicates for each individual condition was calculated. To evaluate the transduction enhancement activity of the tested compounds, average VCNs of cells transduced in the presence of protamine sulfate was set as 1, and enhancement of transduction was expressed as X-fold increase relative to transduction in the presence of protamine sulfate only.

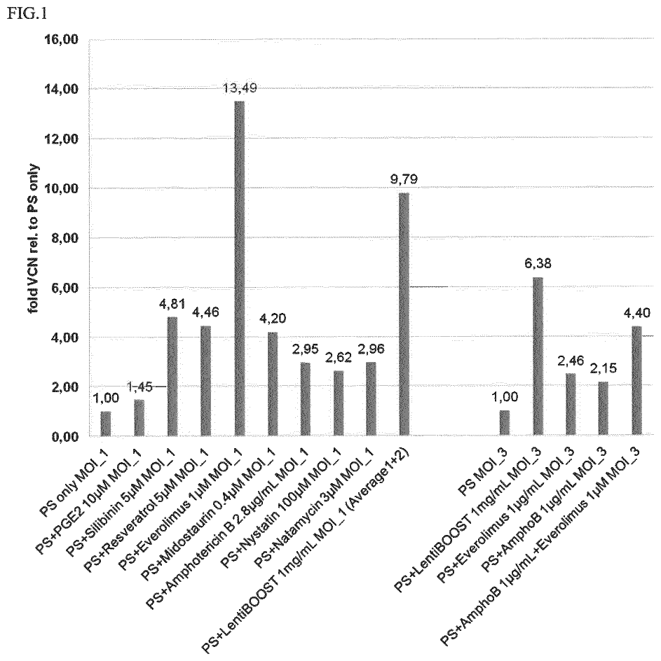

The inventors surprisingly observed the following increases in the efficiency of lentiviral transduction (see FIG. 1):

MOI of 1:

| | |
|---|---|
| Silibinin | (factor 4.8) |
| Resveratrol | (factor 4.5) |
| Everolimus | (factor 13.5) |
| Midostaurin | (factor 4.2) |
| Amphotericin B | (factor 3) |
| Nysatin | (factor 2.6) |
| Natamycin | (factor 3) |
| Lentiboost | (factor 9.8) |

MOI of 3:

| | |
|---|---|
| Everolimus | (factor 2.5) |
| Amphotericin B | (factor 2.2) |
| Everolimus + Amphotericin B | (factor 4.4) |
| Lentiboost | (factor 6.4) |

Supplementation of CD34-positive cells with 300 μM of each deoxyribonucleoside or with 10 μg/mL of BAB-type triblock polymer PCL increased VCN by factor 1.89 or by factor 1.81, respectively, relative to transduction without transduction enhancer.

### Example 2: Transduction in the Presence of Poloxamer F108 and Polybrene (MOI 5)

Human CD34+HSC were transduced by a lentiviral self-inactivating gene therapy vector, comprising a cDNA under control of the miR223 promoter encoding human p47phox, in X-Vivo 20 medium supplemented with 1% human serum albumin, 300 ng/ml SCF, 300 ng/ml Flt3-lig. and 100 ng/ml TPO at a density of 1E6 cells/ml. The transduction process comprised a 2 h pre-stimulation period in presence of poloxamer F108 (1,000 μg/ml) and polybrene (8 μg/ml), followed by 12 h incubation of pre-stimulated cells with the gene therapy vector with a MOI 5 in presence of poloxamer F108 (1,000 μg/ml) and polybrene (8 μg/ml).

### Example 3: Transduction in the Presence of PEG-PCL-PEG Polymer and Midostaurin MOI 5)

Human CD34+HSC were transduced by a lentiviral self-inactivating gene therapy vector, in X-Vivo 20 medium supplemented with 1% human serum albumin, 300 μg/ml SCF, 300 ng/ml Flt3-lig and 100 ng/ml TPO at a density of 1E6 cells/ml. The transduction process comprised a 2 h pre-stimulation period in presence of protamine sulfate (4 μg/mL) plus and PEG-PCL-PEG polymer (4 μg/mL) and midostaurin (0.4 μM) followed by 12 h incubation of pre-stimulated cells with the gene therapy vector with an MOI 5 in presence of protamine sulfate (4 μg/mL) plus and PEG-PCL-PEG polymer (4 μg/mL) and midostaurin (0.4 μM).

### Example 4: Transduction in the Presence of Amphotericin B (MOI 5)

Human CD34+HSC were transduced by a lentiviral self-inactivating gene therapy vector, comprising cDNA, under control of the miR223 promoter, encoding p47phox, in X-Vivo 20 medium supplemented with 1% human serum albumin, 300 ng/ml SCF, 200 ng/ml Flt3-lig and 100 ng/ml TPO at a density of 1E6 cells/ml. The transduction process comprised a 2 h pre-stimulation period in presence of 1 μg/mL Amphotericin B and 4 μg/mL protamine sulfate, followed by 12 h incubation of pre-stimulated cells with the gene therapy vector with an MOI 5 in presence of 1 μg/mL Amphotericin B, and 4 μg/mL protamine sulfate.

### Example 5: Transduction at MOI 10

Commercially available, anonymized human CD34+ haematopoietic stem cells (HSC) were used to test the enhancement of retroviral transduction efficiency by transduction enhancers, or by combinations of transduction enhancers. All experimental conditions were tested in triplicates, and final results were expressed as average of the individual results of each triplicate.

For transduction, a lentiviral self-inactivating (SIN) vector was used, comprising the miR223 promoter as internal promoter, and p47phox encoding cDNA as transgene. Three tests with MOI 10 were conducted independently, in which HSC were retrovirally transduced in presence of either X-Vivo 10 medium or in presence of BESP1366F medium, supplemented with 1% human serum albumin, 300 ng/ml stem cell factor (SCF), 300 ng/ml fins like tyrosine kinase 3 (FLT-3) ligand (Flt3-lig), and 100 ng/ml Thrombopoietin (TPO) at a density of 0.5 E6 cells/cm2, and at a concentration of 1E6 cells/ml.

Before transduction, HSC were cultured in 96 well plates for 22 h with above mentioned media and cytokines, optionally followed by 2 h of pre-stimulation with below mentioned compounds in above mentioned medium without gene therapy vector, after which cells were incubated for transduction without change of medium (with/without compounds) with the lentiviral SIN gene therapy vector at an MOI 10 for 12 h. After the 12 h transduction period, the medium was exchanged by above mentioned medium (including supplements), and cells were transferred to 12-well plates in which they were cultured in a volume of 1 ml of above mentioned medium (including supplements) for 5 to 7 days, depending on cell density. Thereafter, medium was exchanged by fresh medium of abovementioned composition (including supplements), and cells were cultured in 2 ml medium for another 6 days.

At day 12 post transduction, cell numbers were determined, DNA was isolated, and VCN was quantified by qPCR. Cell numbers arising within 12 days from transduced CD34+HSC were compared to cell numbers arising from non-transduced HSC. Transduced cell numbers that did not reach 65% of cell number of non-transduced cells were taken as indication for toxicity of the procedure, using compounds for transduction enhancement. Those samples were excluded from analysis.

For transduction in the presence of potential transduction enhancers, cells were incubated in the presence of:
i) 4, 6 or 8 μg/mL protamine;
ii) 0.5, 0.75, 1, 1.5, 2, or 2.5 mg/ml Lentiboost®;
iii) 0.5, 0.75, or 1 μg/ml Amphotericin B;
iv) 1 or 5 μM Silibinin;
v) 100, 200 or 400 nM Midostaurin;
vi) 4 or 10 μg/ml PCL;
vii) 0.5, 1, or 2 mg/ml Poloxamer F108; or
viii) 10 μg/ml PLA.

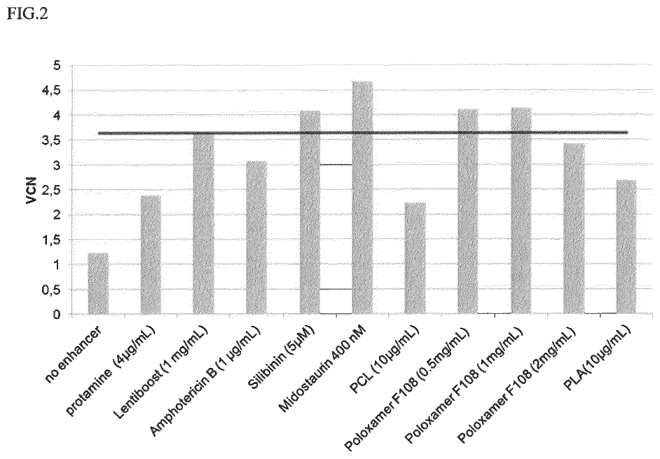

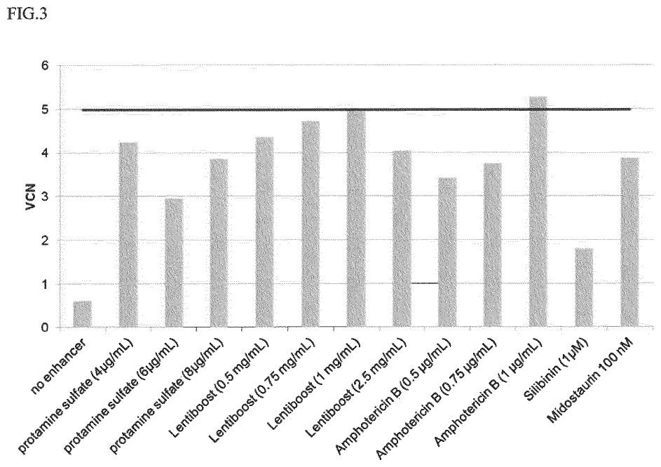

Results are summarized in FIGS. 2 and 3.

Further, combinations of transduction enhancers were tested. For that, transduction in the presence of potential transduction enhancers, cells were incubated in the presence of:
i) 4 μg/mL protamine and 0.75 μg/ml Amphotericin B; or
ii) 4 μg/mL protamine and 10 μg/m1PCL; or
iii) 4 μg/mL protamine and 1 mg/ml Poloxamer F108; or
iv) 1 mg/ml Lentiboost® and 1 μg/ml Amphotericin B; or
v) 1 mg/ml Lentiboost® and 0.75 μg/ml Amphotericin B; or
vi) 1 mg/ml Lentiboost® and 0.5 μg/ml Amphotericin B; or
vii) 1 mg/ml Lentiboost® and 1 μM Silibinin; or
viii) 1 mg/ml Lentiboost® and 100 Midostaurin; or
ix) 1 mg/ml Lentiboost® and 200 nM Midostaurin; or
x) 1 mg/ml Lentiboost® and 400 Midostaurin; or
xi) 1 mg/ml Lentiboost® and 4 μg/mL protamine; or
xii) 1 mg/ml Lentiboost® and 5 μM Silibinin; or
xiii) 1 μg/mL Amphotericin B and 1 mg/ml Poloxamer F108; or
xiv) 1 μg/mL Amphotericin B and 10 μg/ml PCL; or
xv) 5 μM Silibinin and 10 μg/ml PCL; or
xvi) 5 μM Silibinin and 1 mg/ml Poloxamer F108; or
xvii) 400 nM Midostaurin and 1 mg/ml Poloxamer F108; or
xviii) 400 Midostaurin and 10 μg/ml PCL.

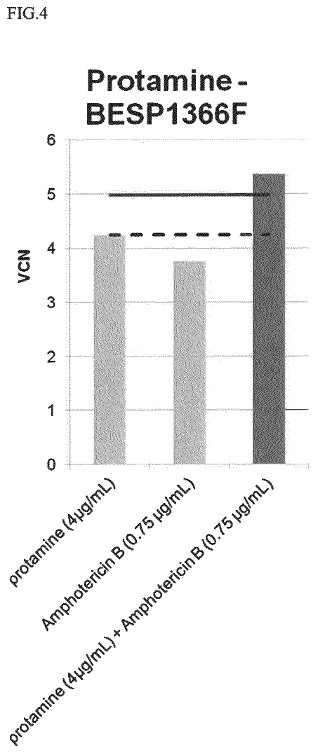

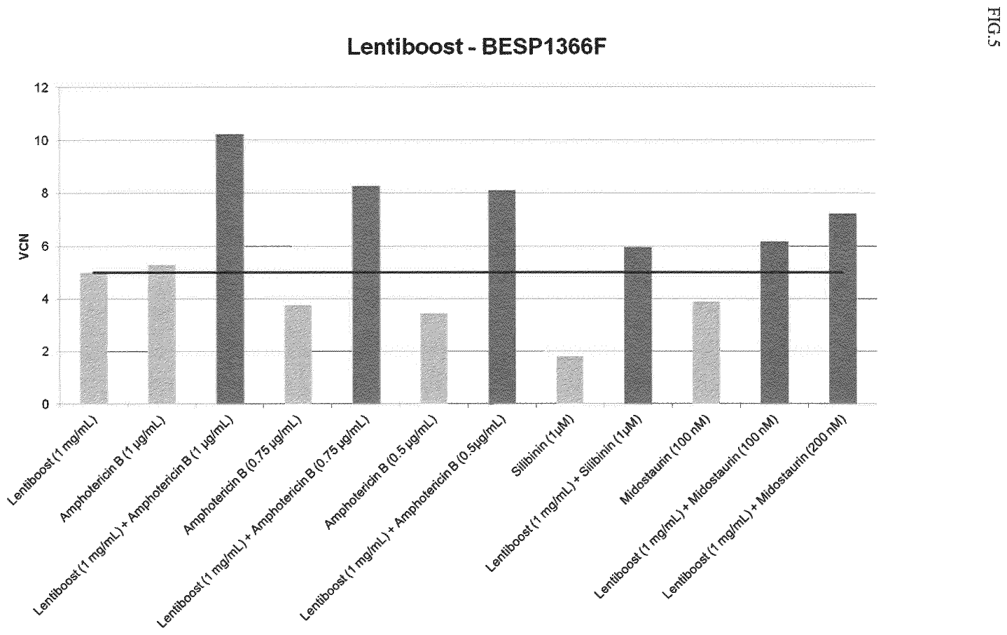

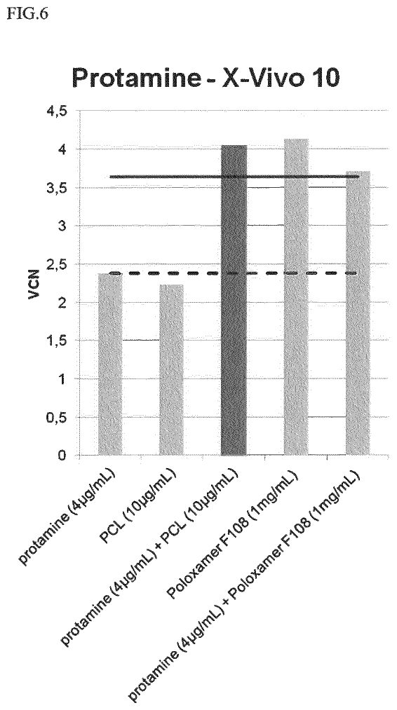

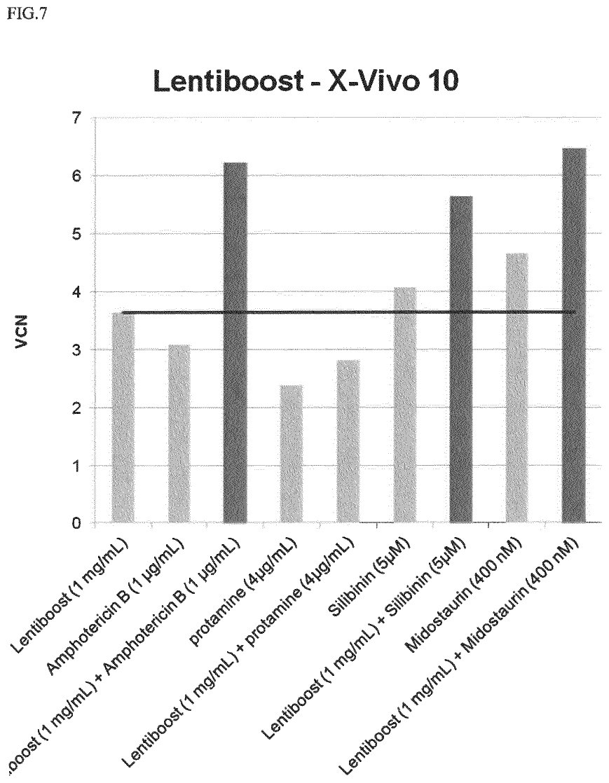

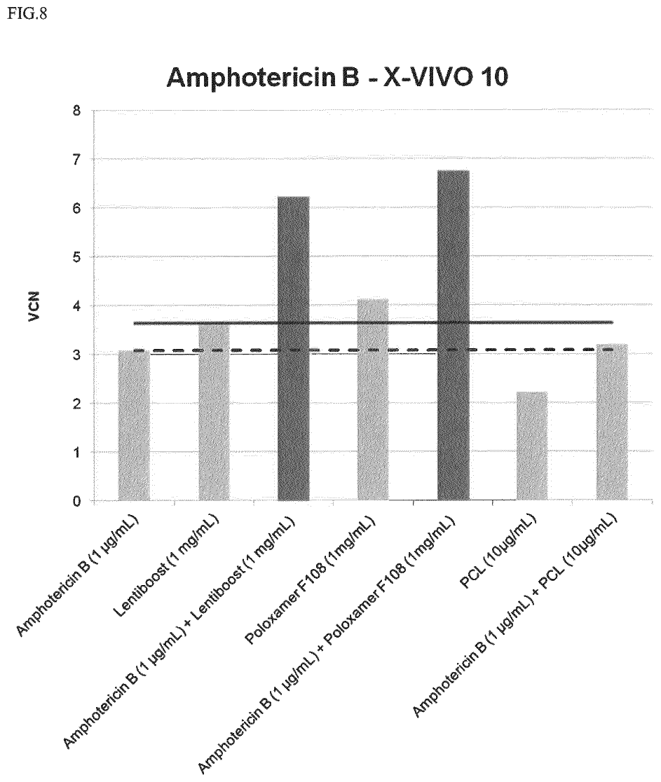

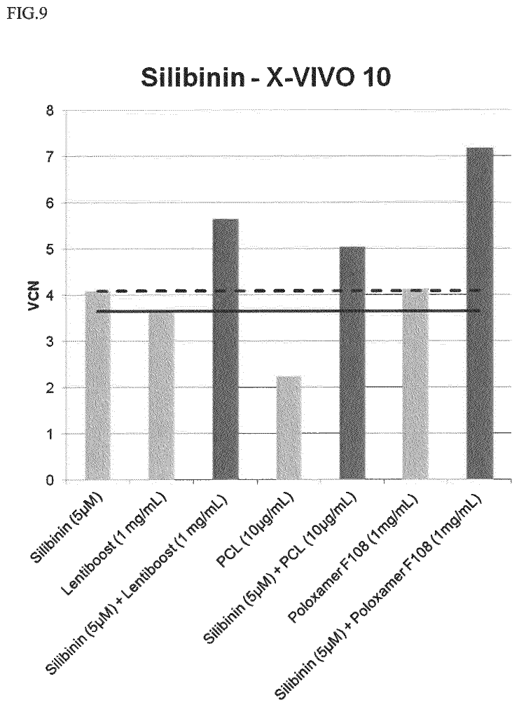

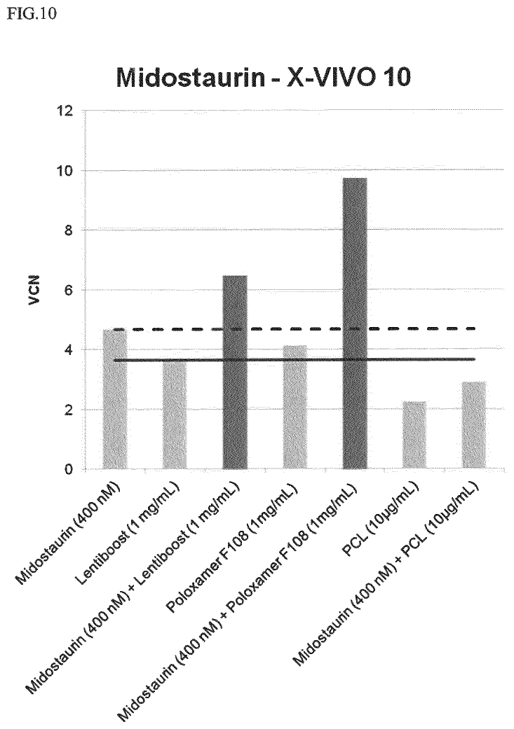

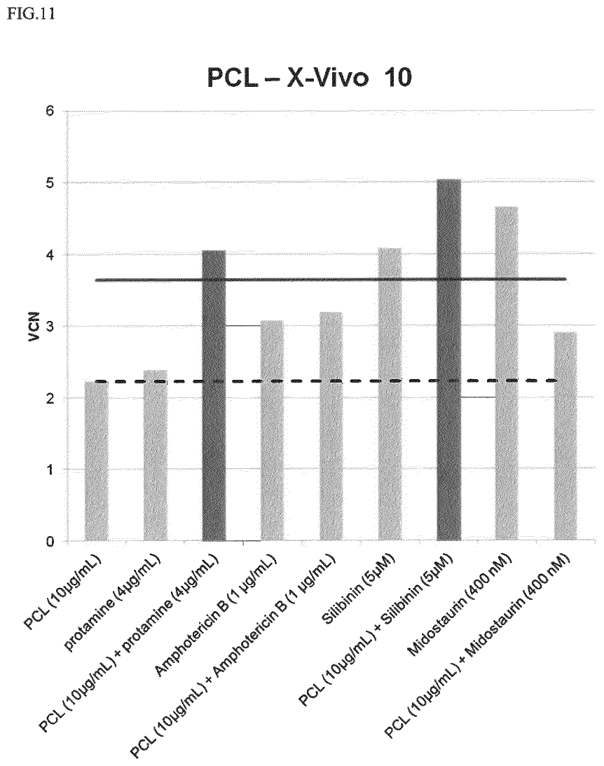

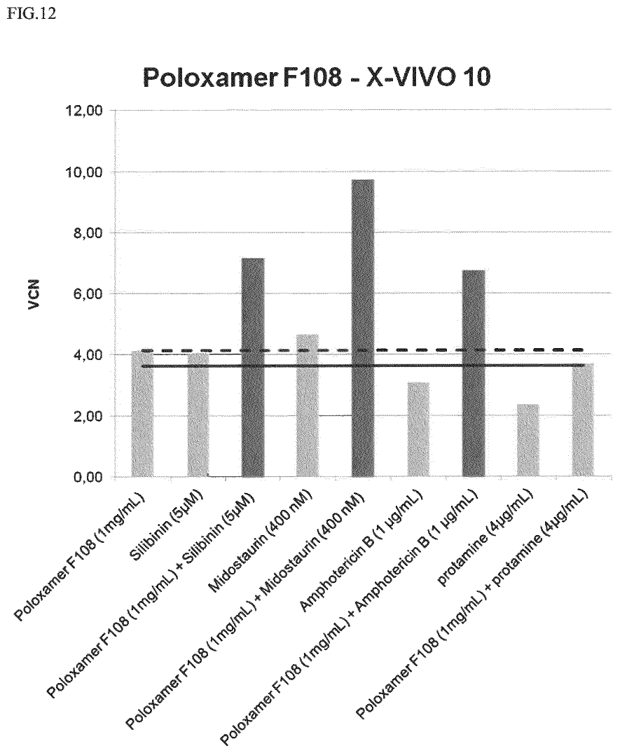

Results for the combinations of transduction enhancers are summarized in FIGS. 4-12.

### Example 5: Transduction at MOI 20

Human CD34+HSC were transduced by a lentiviral self-inactivating gene therapy vector in X-Vivo 20 medium supplemented with 1% human serum albumin, 300 ng/ml SCF, 300 ng/ml Flt3-lig and 100 ng/ml TPO at a density of 1E6 cells/ml. The transduction process comprised a 2 h pre-stimulation period in presence of 1% DMSO, followed by 16 h incubation of pre-stimulated cells with the gene therapy vector with an MOI 20 in presence of 1% DMSO.

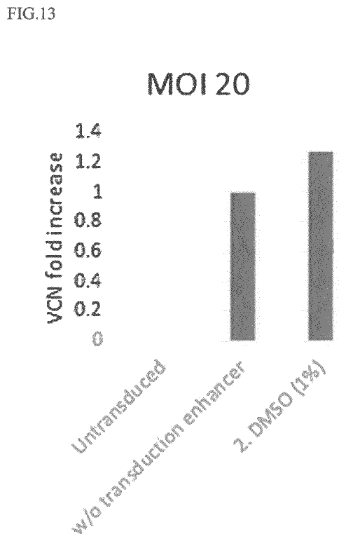

The vector copy number (VCN) in the absence of DMSO was 0.915 and 1.16 in the presence of DMSO. DMSO was thus shown to have a transduction enhancing effect. The results are summarized in FIG. 13.

1. A method for transducing a target cell, the method comprising a step of contacting a target cell with a retroviral vector and a compound capable of enhancing transduction efficiency or a combination of such compounds, wherein the target cell is pre- and/or co-stimulated by pre- and/or co-incubation with said transduction enhancing compound or a combination of transduction enhancing compounds prior to and/or during contacting the target cell with the retroviral vector.

2. The method of claim 1, wherein the transduction enhancing compound is Amphotericin B, in particular wherein the target cell is contacted with Amphotericin B during the pre-incubation and/or co-incubation step at a concentration of about 0.05 to about 500 μM, in particular at a concentration of about 0.1 to about 10 μM.

3. The method of claim 2, wherein Amphotericin B is used in combination with one or more additional transduction enhancing compounds.

4. The method of claim 3, wherein the additional transduction enhancing compound is a protamine salt, in particular wherein the protamine salt is protamine chloride or protamine sulfate.

5. The method of claim 4, wherein the target cell is contacted with the protamine salt during the pre-incubation and/or co-incubation step at a concentration of about 0.05 μg/mL to about 25 mg/mL, in particular at a concentration of about 0.1 μg/mL to about 10 μg/mL.

6. The method claim 3, wherein the one or more additional transduction enhancing compound is selected from the group consisting of:
Lentiboost®, in particular at a concentration between 0.1 mg/ml and 5,000 mg/ml;
poloxamer F108, in particular at a concentration between 011 mg/ml and 5,000 mg/ml;
Silibinin, in particular at a concentration between 0.05 μM and 500 μM;
Midostaurin, in particular at a concentration between 2 nM and 500,000 nM;
PEG-PLA-PEG, in particular at a concentration between 1 ng/ml and 5,000 ng/ml;
PEG-PLGA-PEG, in particular at a concentration between 1 ng/ml and 5,000 ng/ml;
PEG-PCL-PEG, in particular at a concentration between 1 ng/ml and 5,000 ng/ml;
Nystatin, in particular at a concentration between 0.1 and 1000 μM;
Natamycin, in particular at a concentration between 0.05 and 500 μM;
Ruxolitinib, in particular at a concentration between 0.01 and 10,000 μM;
Fludarabine, in particular at a concentration between 0.01 and 10,000 μM;
Everolimus, in particular at a concentration between 0.1 and 10 μM;
Resveratrol, in particular at a concentration between 0.1 and 25 μM;
Prostaglandin E, in particular at a concentration between I and 100 μM;
Deoxyribonucleosides, in particular at a concentration between 0.1 mM and 1OmM of each nucleoside;
DMSO, in particular at a concentration between 0.1 and 10% (v/v); and/or
any combination thereof;
in particular wherein the one or more additional transduction enhancing compound is selected from the group consisting of: Lentiboost®, poloxamer F108 and/or a PEG-PCL-PEG polymer.

7. The method according to claim 1, wherein the transduction enhancing compound is selected from a group consisting of:
Silibinin, in particular at a concentration between 0.05 μM and 500 μM;
Midostaurin, in particular at a concentration between 2 nM and 500,000 nM;
Nystatin, in particular at a concentration between 0.1 and 1000 μM;
Natamycin, in particular at a concentration between 0.05 and 500 μM;
a PEG-PCL-PEG polymer, in particular at a concentration between 1 μg/ml and 5,000 μg/ml
a PEG-PLGA-PEG polymer, in particular at a concentration between 1 μg/ml and 5,000 μg/ml
a PEG-PLA-PEG polymer, in particular at a concentration between 1 μg/ml and 5,000 μg/ml
DMSO, in particular at a concentration between 0.1 and 10% (v/v); and/or
any combination thereof.

8. The method according to claim 7, wherein the transduction enhancing compound is used in combination with one or more additional transduction enhancing compounds, in particular wherein the one or more additional transduction enhancing compound is selected from a group consisting of:
Lentiboost®, in particular at a concentration between 0.1 mg/ml and 5,000 mg/ml;
poloxamer F108, in particular at a concentration between O.lmg/ml and 5,000 mg/ml;
Everolimus, in particular at a concentration between 0.1 and 10 μM;
Resveratrol, in particular at a concentration between 0.1 and 25 μM;
Prostaglandin E, in particular at a concentration between 1 and 100 μM;
A protamine salt, in particular at a concentration between 0.05 μg/mL and 25 μg/mL;
Desoxyribonucleosides, in particular at a concentration between 0.1 mM and 10 mM of each nucleoside; and/or
any combination thereof.

9. The method of claim 1, wherein the target cell is co-incubated with the transduction enhancing compound or the combination of transduction enhancing compounds during contacting the target cell with a retroviral vector for a period between about 8 hours and about 48 hours, particularly between about 10 hours and about 24 hours, but particularly about 12 hours.

10. The method of claim 1, wherein the target cell is pre-incubated with the transduction enhancing compound or the combination of transduction enhancing compounds prior to contacting the target cell with a retroviral vector for a period between about 0.5 hours and about 10 hours, particularly between about 1 hour and about 5 hours, but particularly about 2 hours.

11. The method of claim 1, wherein the target cell is a mammalian cell, in particular a human cell.

12. The method of claim 1, wherein the target cell is a cell selected from the group consisting of a lymphocyte, a tumor cell, a lymphoid lineage cell, a neuronal cell, an epithelial cell, a keratinocyte, an endothelial cell, a primary cell, a T cell, a haematopoietic cell, and a stem cell.

13. The method of claim 12, wherein the haematopoietic cell is a haematopoietic stein cell, a haematopoietic progenitor cell, a CD34+ cell, a monocyte, a macrophage, a tissue resident macrophage, a microglial cell, a keratinocyte or a dendritic.

14. The method of claim 12, wherein the T cell is characterized by surface presentation of CD3, CD4 and/or CD8.

15. The method of claim 1, wherein the retroviral vector is a lentiviral vector, in particular a self-inactivating lentiviral vector.

16. The method of claim 1, wherein the vector comprises a transgene under control of a promoter, in particular wherein the transgene encodes a therapeutic protein or a chimeric antigen receptor (CAR).
